SEQUENCE LISTING (24 sequences)

SEQ ID NO: 1 - DNA, 1011 bp, Artificial Sequence
  Synthetic polynucleotide
ctgaaaagtcacaagacttaacagaagtaagtcatatgatcaatcaaataagctttgcgtttcacgtccgtaatttgggtgtctcttgttaagtgaaggcgttgtctgtatcttcattaacttatatctcttgatagtatttcttgaaaaagttcaccatggctttgagcgacgttgatgtacaaaagcagatcaagcatatgatggctttcattgaacaagaggcaaacgaaaaggcagaagaaatcgatgccaaggccgaggaagaatttaatattgaaaaggggcgccttgttcagcaacaacgtctcaagattatggaatattatgagaagaaagagaaacaggtcgaactccagaaaaaaatccaatcgtctaacatgttgaatcaggctcgattgaaagtattgaaggttagggaagatcacgttcgtaccgtactagaggaggcgcgtaaacgacttggtcaggtcacaaacgaccagggaaaatattcccaaatcctggaaagcctcattttgcagggattatatcagctttttgagaaagatgttaccattcgagttcggccccaggaccgagaactggtcaaatccatcattcccaccgtcacgaacaagtataaagatgccaccggtaaggacatccatctgaaaattgatgacgaaatccatctgtcccaagaaaccaccgggggaatcgacctgctggcgcagaaaaacaaaatcaagatcagcaatactatggaggctcgtctggagctgatttcgcagcagttggtgccacaaatccgtacggctctgtttggacgtaacgtcaacaggaaattcaccgattaggcatttatttggtggtcagttttgcatgttgtattctgcaattcgctgtaattgaaacactaaagtgtttcggaaagttttagctgaattatgttcattaggatatcgtggttactggaacattaatctgtatataacatattccttgttaatagtattttttttgttttat

SEQ ID NO: 2 - RNA, 1011 nt, Artificial Sequence
  Synthetic polynucleotide
cugaaaagucacaagacuuaacagaaguaagucauaugaucaaucaaauaagcuuugcguuucacguccguaauuugggugucucuuguuaagugaaggcguugucuguaucuucauuaacuuauaucucuugauaguauuucuugaaaaaguucaccauggcuuugagcgacguugauguacaaaagcagaucaagcauaugauggcuuucauugaacaagaggcaaacgaaaaggcagaagaaaucgaugccaaggccgaggaagaauuuaauauugaaaaggggcgccuuguucagcaacaacgucucaagauuauggaauauuaugagaagaaagagaaacaggucgaacuccagaaaaaaauccaaucgucuaacauguugaaucaggcucgauugaaaguauugaagguuagggaagaucacguucguaccguacuagaggaggcgcguaaacgacuuggucaggucacaaacgaccagggaaaauauucccaaauccuggaaagccucauuuugcagggauuauaucagcuuuuugagaaagauguuaccauucgaguucggccccaggaccgagaacuggucaaauccaucauucccaccgucacgaacaaguauaaagaugccaccgguaaggacauccaucugaaaauugaugacgaaauccaucugucccaagaaaccaccgggggaaucgaccugcuggcgcagaaaaacaaaaucaagaucagcaauacuauggaggcucgucuggagcugauuucgcagcaguuggugccacaaauccguacggcucuguuuggacguaacgucaacaggaaauucaccgauuaggcauuuauuugguggucaguuuugcauguuguauucugcaauucgcuguaauugaaacacuaaaguguuucggaaaguuuuagcugaauuauguucauuaggauaucgugguuacuggaacauuaaucuguauauaacauauuccuuguuaauaguauuuuuuuuguuuuau

SEQ ID NO: 3 - RNA, 400 nt, Artificial Sequence
  Synthetic polynucleotide
ucauaugaucaaucaaauaagcuuugcguuucacguccguaauuugggugucucuuguuaagugaaggcguugucuguaucuucauuaacuuauaucucuugauaguauuucuugaaaaaguucaccauggcuuugagcgacguugauguacaaaagcagaucaagcauaugauggcuuucauugaacaagaggcaaacgaaaaggcagaagaaaucgaugccaaggccgaggaagaauuuaauauugaaaaggggcgccuuguucagcaacaacgucucaagauuauggaauauuaugagaagaaagagaaacaggucgaacuccagaaaaaaauccaaucgucuaacauguugaaucaggcucgauugaaaguauugaagguuagggaagaucacguu

SEQ ID NO: 4 - RNA, 408 nt, Artificial Sequence
  Synthetic polynucleotide
cguaccguacuagaggaggcgcguaaacgacuuggucaggucacaaacgaccagggaaaauauucccaaauccuggaaagccucauuuugcagggauuauaucagcuuuuugagaaagauguuaccauucgaguucggccccaggaccgagaacuggucaaauccaucauucccaccgucacgaacaaguauaaagaugccaccgguaaggacauccaucugaaaauugaugacgaaauccaucugucccaagaaaccaccgggggaaucgaccugcuggcgcagaaaaacaaaaucaagaucagcaauacuauggaggcucgucuggagcugauuucgcagcaguuggugccacaaauccguacggcucuguuuggacguaacgucaacaggaaauucaccgauuag

SEQ ID NO: 5 - RNA, 1011 nt, Artificial Sequence
  Synthetic polynucleotide
auaaaacaaaaaaaauacuauuaacaaggaauauguuauauacagauuaauguuccaguaaccacgauauccuaaugaacauaauucagcuaaaacuuuccgaaacacuuuaguguuucaauuacagcgaauugcagaauacaacaugcaaaacugaccaccaaauaaaugccuaaucggugaauuuccuguugacguuacguccaaacagagccguacggauuuguggcaccaacugcugcgaaaucagcuccagacgagccuccauaguauugcugaucuugauuuuguuuuucugcgccagcaggucgauucccccggugguuucuugggacagauggauuucgucaucaauuuucagauggauguccuuaccgguggcaucuuuauacuuguucgugacggugggaaugauggauuugaccaguucucgguccuggggccgaacucgaaugguaacaucuuucucaaaaagcugauauaaucccugcaaaaugaggcuuuccaggauuugggaauauuuucccuggucguuugugaccugaccaagucguuuacgcgccuccucuaguacgguacgaacgugaucuucccuaaccuucaauacuuucaaucgagccugauucaacauguuagacgauuggauuuuuuucuggaguucgaccuguuucucuuucuucucauaauauuccauaaucuugagacguuguugcugaacaaggcgccccuuuucaauauuaaauucuuccucggccuuggcaucgauuucuucugccuuuucguuugccucuuguucaaugaaagccaucauaugcuugaucugcuuuuguacaucaacgucgcucaaagccauggugaacuuuuucaagaaauacuaucaagagauauaaguuaaugaagauacagacaacgccuucacuuaacaagagacacccaaauuacggacgugaaacgcaaagcuuauuugauugaucauaugacuuacuucuguuaagucuugugacuuuucag

SEQ ID NO: 6 - RNA, 400 nt, Artificial Sequence
  Synthetic polynucleotide
aacgugaucuucccuaaccuucaauacuuucaaucgagccugauucaacauguuagacgauuggauuuuuuucuggaguucgaccuguuucucuuucuucucauaauauuccauaaucuugagacguuguugcugaacaaggcgccccuuuucaauauuaaauucuuccucggccuuggcaucgauuucuucugccuuuucguuugccucuuguucaaugaaagccaucauaugcuugaucugcuuuuguacaucaacgucgcucaaagccauggugaacuuuuucaagaaauacuaucaagagauauaaguuaaugaagauacagacaacgccuucacuuaacaagagacacccaaauuacggacgugaaacgcaaagcuuauuugauugaucauauga

SEQ ID NO: 7 - RNA, 408 nt, Artificial Sequence
  Synthetic polynucleotide
cuaaucggugaauuuccuguugacguuacguccaaacagagccguacggauuuguggcaccaacugcugcgaaaucagcuccagacgagccuccauaguauugcugaucuugauuuuguuuuucugcgccagcaggucgauucccccggugguuucuugggacagauggauuucgucaucaauuuucagauggauguccuuaccgguggcaucuuuauacuuguucgugacggugggaaugauggauuugaccaguucucgguccuggggccgaacucgaaugguaacaucuuucucaaaaagcugauauaaucccugcaaaaugaggcuuuccaggauuugggaauauuuucccuggucguuugugaccugaccaagucguuuacgcgccuccucuaguacgguacg

SEQ ID NO: 8 - DNA, 856 bp, Artificial Sequence
  Synthetic polynucleotide
cagacgtcaaaatctgaatacctatacctctgtgattctgtgattttgttttttagttgcaagtaatcatggcttcggagcggtatagtttttcgctgacaactttcagtccatctggaaaactagttcaaattgaatatgccctagctgctgtagccgctggagctccttcagtgggcattaaagcttcaaatggtgtagttatcgccacagaaaacaaacataagtcgatcctctatgaagaacacagtgttcataaagtggaaatgattacaaaacatataggaatgatatattctggtatgggacctgattatcgcttgttagtgaaacaagctcgtaaaatggcccaacagtattatctagtttatcaagagcctataccaacagttcaactcgttcaacgagttgccactgttatgcaagaatatactcagtccggaggagttaggccgtttggggtttcattattgatatgtggttgggacagtgaacgaccatacttatttcaatgtgatccatcaggagcttattttgcctggaaagctactgccatgggcaagaatttcatcaatggaaaaacatttttggaaaaaagatatagcgaggatttggaacttgatgacgcagtacacacagcaattctgacgttgaaggagagttttgaaggccaaatgacagcggacaacattgaagtgggaatttgtgatgaagcaggattcaggaggctagatccctctcatgtgaaggattacctagctaatattccataaggcatttaggttatataacaagatttctcttaattttttatgaaactcatgtttcacttgaataaaaccggatttgaaggaaa

SEQ ID NO: 9 - DNA, 1093 bp, Artificial Sequence
  Synthetic polynucleotide
atatatttatttgggaactttcgatgtttagagacgatgtaatgtccatgtcaatgaccaatgcaaataaaagataccatgtaggtatatgctctgtgattccatgatttttccgatacataataaagttcaaatacttccgtttatttaaggcagttattttattcacaaaaattttgtgaaatttgaacaagccatgctgaacctttttcaatgccttaatgagaaaaatctttttgacgtcttgagaagagcaaataaaagtcagacgtcaaaatctgaatacctatacctctgtgattctgtgattttgttttttagttgcaagtaatcatggcttcggagcggtatagtttttcgctgacaactttcaggtgattataattcaattattgaaggaacagtattcatattgttttaattttcagtccatctggaaaactagttcaaattgaatatgccctagctgctgtagccgctggagctccttcagtgggcattaaaggtatttaacctctacctagtgaatgatacatcattttcatgcacataattaaatttttcttatagcttcaaatggtgtagttatcgccacagaaaacaaacataagtcgatcctctatgaagaacacagtgttcataaagtggaaatgattacaaaacatataggaatgatatattctggtatgggacctgattatcgcttgttagtgaaacaagctcgtaaaatggcccaacagtattatctagtttatcaagagcctataccaacagttcaactcgttcaacgagttgccactgttatgcaagaatatactcagtccggaggagttaggccgtttggggtttcattattgatatgtggttgggacagtgaacgaccatacttatttcaatgtgatccatcaggagcttattttgcctggaaagctactgccatgggcaagaatttcatcaatggaaaaacatttttggaaaaaaggtaacagcaaatttaaaaaacatgaaaatagaaaataatcaaacaaatctcataatatttatagacgtagacaaattttgaatgctaatgacgggaaagtagtttttttct

SEQ ID NO: 10 - RNA, 856 nt, Artificial Sequence
  Synthetic polynucleotide
cagacgucaaaaucugaauaccuauaccucugugauucugugauuuuguuuuuuaguugcaaguaaucauggcuucggagcgguauaguuuuucgcugacaacuuucaguccaucuggaaaacuaguucaaauugaauaugcccuagcugcuguagccgcuggagcuccuucagugggcauuaaagcuucaaaugguguaguuaucgccacagaaaacaaacauaagucgauccucuaugaagaacacaguguucauaaaguggaaaugauuacaaaacauauaggaaugauauauucugguaugggaccugauuaucgcuuguuagugaaacaagcucguaaaauggcccaacaguauuaucuaguuuaucaagagccuauaccaacaguucaacucguucaacgaguugccacuguuaugcaagaauauacucaguccggaggaguuaggccguuugggguuucauuauugauaugugguugggacagugaacgaccauacuuauuucaaugugauccaucaggagcuuauuuugccuggaaagcuacugccaugggcaagaauuucaucaauggaaaaacauuuuuggaaaaaagauauagcgaggauuuggaacuugaugacgcaguacacacagcaauucugacguugaaggagaguuuugaaggccaaaugacagcggacaacauugaagugggaauuugugaugaagcaggauucaggaggcuagaucccucucaugugaaggauuaccuagcuaauauuccauaaggcauuuagguuauauaacaagauuucucuuaauuuuuuaugaaacucauguuucacuugaauaaaaccggauuugaaggaaa

SEQ ID NO: 11 - RNA, 1093 nt, Artificial Sequence
  Synthetic polynucleotide
auauauuuauuugggaacuuucgauguuuagagacgauguaauguccaugucaaugaccaaugcaaauaaaagauaccauguagguauaugcucugugauuccaugauuuuuccgauacauaauaaaguucaaauacuuccguuuauuuaaggcaguuauuuuauucacaaaaauuuugugaaauuugaacaagccaugcugaaccuuuuucaaugccuuaaugagaaaaaucuuuuugacgucuugagaagagcaaauaaaagucagacgucaaaaucugaauaccuauaccucugugauucugugauuuuguuuuuuaguugcaaguaaucauggcuucggagcgguauaguuuuucgcugacaacuuucaggugauuauaauucaauuauugaaggaacaguauucauauuguuuuaauuuucaguccaucuggaaaacuaguucaaauugaauaugcccuagcugcuguagccgcuggagcuccuucagugggcauuaaagguauuuaaccucuaccuagugaaugauacaucauuuucaugcacauaauuaaauuuuucuuauagcuucaaaugguguaguuaucgccacagaaaacaaacauaagucgauccucuaugaagaacacaguguucauaaaguggaaaugauuacaaaacauauaggaaugauauauucugguaugggaccugauuaucgcuuguuagugaaacaagcucguaaaauggcccaacaguauuaucuaguuuaucaagagccuauaccaacaguucaacucguucaacgaguugccacuguuaugcaagaauauacucaguccggaggaguuaggccguuugggguuucauuauugauaugugguugggacagugaacgaccauacuuauuucaaugugauccaucaggagcuuauuuugccuggaaagcuacugccaugggcaagaauuucaucaauggaaaaacauuuuuggaaaaaagguaacagcaaauuuaaaaaacaugaaaauagaaaauaaucaaacaaaucucauaauauuuauagacguagacaaauuuugaaugcuaaugacgggaaaguaguuuuuuucu

SEQ ID NO: 12 - RNA, 385 nt, Artificial Sequence
  Synthetic polynucleotide
uaaucauggcuucggagcgguauaguuuuucgcugacaacuuucaggugauuauaauucaauuauugaaggaacaguauucauauuguuuuaauuuucaguccaucuggaaaacuaguucaaauugaauaugcccuagcugcuguagccgcuggagcuccuucagugggcauuaaagguauuuaaccucuaccuagugaaugauacaucauuuucaugcacauaauuaaauuuuucuuauagcuucaaaugguguaguuaucgccacagaaaacaaacauaagucgauccucuaugaagaacacaguguucauaaaguggaaaugauuacaaaacauauaggaaugauauauucugguaugggaccugauuaucgcuuguuagug

SEQ ID NO: 13 - RNA, 388 nt, Artificial Sequence
  Synthetic polynucleotide
cagacgucaaaaucugaauaccuauaccucugugauucugugauuuuguuuuuuaguugcaaguaaucauggcuucggagcgguauaguuuuucgcugacaacuuucaguccaucuggaaaacuaguucaaauugaauaugcccuagcugcuguagccgcuggagcuccuucagugggcauuaaagcuucaaaugguguaguuaucgccacagaaaacaaacauaagucgauccucuaugaagaacacaguguucauaaaguggaaaugauuacaaaacauauaggaaugauauauucugguaugggaccugauuaucgcuuguuagugaaacaagcucguaaaauggcccaacaguauuaucuaguuuaucaagagccuauaccaac

SEQ ID NO: 14 - RNA, 856 nt, Artificial Sequence
  Synthetic polynucleotide
uuuccuucaaauccgguuuuauucaagugaaacaugaguuucauaaaaaauuaagagaaaucuuguuauauaaccuaaaugccuuauggaauauuagcuagguaauccuucacaugagagggaucuagccuccugaauccugcuucaucacaaauucccacuucaauguuguccgcugucauuuggccuucaaaacucuccuucaacgucagaauugcuguguguacugcgucaucaaguuccaaauccucgcuauaucuuuuuuccaaaaauguuuuuccauugaugaaauucuugcccauggcaguagcuuuccaggcaaaauaagcuccugauggaucacauugaaauaaguauggucguucacugucccaaccacauaucaauaaugaaaccccaaacggccuaacuccuccggacugaguauauucuugcauaacaguggcaacucguugaacgaguugaacuguugguauaggcucuugauaaacuagauaauacuguugggccauuuuacgagcuuguuucacuaacaagcgauaaucaggucccauaccagaauauaucauuccuauauguuuuguaaucauuuccacuuuaugaacacuguguucuucauagaggaucgacuuauguuuguuuucuguggcgauaacuacaccauuugaagcuuuaaugcccacugaaggagcuccagcggcuacagcagcuagggcauauucaauuugaacuaguuuuccagauggacugaaaguugucagcgaaaaacuauaccgcuccgaagccaugauuacuugcaacuaaaaaacaaaaucacagaaucacagagguauagguauucagauuuugacgucug

SEQ ID NO: 15 - RNA, 1093 nt, Artificial Sequence
  Synthetic polynucleotide
agaaaaaaacuacuuucccgucauuagcauucaaaauuugucuacgucuauaaauauuaugagauuuguuugauuauuuucuauuuucauguuuuuuaaauuugcuguuaccuuuuuuccaaaaauguuuuuccauugaugaaauucuugcccauggcaguagcuuuccaggcaaaauaagcuccugauggaucacauugaaauaaguauggucguucacugucccaaccacauaucaauaaugaaaccccaaacggccuaacuccuccggacugaguauauucuugcauaacaguggcaacucguugaacgaguugaacuguugguauaggcucuugauaaacuagauaauacuguugggccauuuuacgagcuuguuucacuaacaagcgauaaucaggucccauaccagaauauaucauuccuauauguuuuguaaucauuuccacuuuaugaacacuguguucuucauagaggaucgacuuauguuuguuuucuguggcgauaacuacaccauuugaagcuauaagaaaaauuuaauuaugugcaugaaaaugauguaucauucacuagguagagguuaaauaccuuuaaugcccacugaaggagcuccagcggcuacagcagcuagggcauauucaauuugaacuaguuuuccagauggacugaaaauuaaaacaauaugaauacuguuccuucaauaauugaauuauaaucaccugaaaguugucagcgaaaaacuauaccgcuccgaagccaugauuacuugcaacuaaaaaacaaaaucacagaaucacagagguauagguauucagauuuugacgucugacuuuuauuugcucuucucaagacgucaaaaagauuuuucucauuaaggcauugaaaaagguucagcauggcuuguucaaauuucacaaaauuuuugugaauaaaauaacugccuuaaauaaacggaaguauuugaacuuuauuauguaucggaaaaaucauggaaucacagagcauauaccuacaugguaucuuuuauuugcauuggucauugacauggacauuacaucgucucuaaacaucgaaaguucccaaauaaauauau

SEQ ID NO: 16 - RNA, 385 nt, Artificial Sequence
  Synthetic polynucleotide
cacuaacaagcgauaaucaggucccauaccagaauauaucauuccuauauguuuuguaaucauuuccacuuuaugaacacuguguucuucauagaggaucgacuuauguuuguuuucuguggcgauaacuacaccauuugaagcuauaagaaaaauuuaauuaugugcaugaaaaugauguaucauucacuagguagagguuaaauaccuuuaaugcccacugaaggagcuccagcggcuacagcagcuagggcauauucaauuugaacuaguuuuccagauggacugaaaauuaaaacaauaugaauacuguuccuucaauaauugaauuauaaucaccugaaaguugucagcgaaaaacuauaccgcuccgaagccaugauua

SEQ ID NO: 17 - RNA, 388 nt, Artificial Sequence
  Synthetic polynucleotide
guugguauaggcucuugauaaacuagauaauacuguugggccauuuuacgagcuuguuucacuaacaagcgauaaucaggucccauaccagaauauaucauuccuauauguuuuguaaucauuuccacuuuaugaacacuguguucuucauagaggaucgacuuauguuuguuuucuguggcgauaacuacaccauuugaagcuuuaaugcccacugaaggagcuccagcggcuacagcagcuagggcauauucaauuugaacuaguuuuccagauggacugaaaguugucagcgaaaaacuauaccgcuccgaagccaugauuacuugcaacuaaaaaacaaaaucacagaaucacagagguauagguauucagauuuugacgucug

SEQ ID NO: 18 - DNA, 1010 bp, Artificial Sequence
  Synthetic polynucleotide
cgtcattctgccaacttctcctttactgtcaaccataagtaacagaactgttactatcaaattaaaaaactcttaaaataataattgggcgattttaaagacttttctgtagtgaatatcaactttacatattttttgtgggataattggtcgtttccaacgttagttcaaattacaaaaatgttcatctgggattggtttaccggtgtacttggttacttgggattatggaagaaatcaggcaagctgcttttcctaggcctggacaatgctggaaaaacaactttactgcatatgctcaaagatgataggctggcacagcatcttcccactttacatcctagtaaggctcttaacatttttcataatgaaatgtccagtccacatcacatgcttcaccttcatttttgtagaagatcatacccccgaaaaacattaaggaacttcgattcctccacaaaaaatgtctccaaatatgctaaccttatttttctctctacagcaaggcgagtgtggaaggactacttccctgctgttgacgccattgtctttttagtagatgccaatgataggaacagattcaaagagagtaaacaagaattggattcgctactcacagacgagactctttcgaattgtccggttcttatattaggtaacaaaattgatttgcctcaagcagcttcggaagacgaactaagaaattactatgccttgtatggccaaacaactggaaagggaaaggtggcaaggtccgatttgccaggacgtcccctcgagctcttcatgtgctccattctgaagaggcagggatacggagaaggtttccgttggctggcccagtacatcgactgaagtttttttgtgcgagtggattcgttaaggtgtacctacacggtattctttcatgaacaaggtttttctatatcaattcatatgtgacctttttttttcggtataattggttttttatcgtgtagtatatataaaaaaaatagttttaatagtag

SEQ ID NO: 19 - RNA, 1010 nt, Artificial Sequence
  Synthetic polynucleotide
cgucauucugccaacuucuccuuuacugucaaccauaaguaacagaacuguuacuaucaaauuaaaaaacucuuaaaauaauaauugggcgauuuuaaagacuuuucuguagugaauaucaacuuuacauauuuuuugugggauaauuggucguuuccaacguuaguucaaauuacaaaaauguucaucugggauugguuuaccgguguacuugguuacuugggauuauggaagaaaucaggcaagcugcuuuuccuaggccuggacaaugcuggaaaaacaacuuuacugcauaugcucaaagaugauaggcuggcacagcaucuucccacuuuacauccuaguaaggcucuuaacauuuuucauaaugaaauguccaguccacaucacaugcuucaccuucauuuuuguagaagaucauacccccgaaaaacauuaaggaacuucgauuccuccacaaaaaaugucuccaaauaugcuaaccuuauuuuucucucuacagcaaggcgaguguggaaggacuacuucccugcuguugacgccauugucuuuuuaguagaugccaaugauaggaacagauucaaagagaguaaacaagaauuggauucgcuacucacagacgagacucuuucgaauuguccgguucuuauauuagguaacaaaauugauuugccucaagcagcuucggaagacgaacuaagaaauuacuaugccuuguauggccaaacaacuggaaagggaaagguggcaagguccgauuugccaggacguccccucgagcucuucaugugcuccauucugaagaggcagggauacggagaagguuuccguuggcuggcccaguacaucgacugaaguuuuuuugugcgaguggauucguuaagguguaccuacacgguauucuuucaugaacaagguuuuucuauaucaauucauaugugaccuuuuuuuuucgguauaauugguuuuuuaucguguaguauauauaaaaaaaauaguuuuaauaguag

SEQ ID NO: 20 - RNA, 600 nt, Artificial Sequence
  Synthetic polynucleotide
gcaauuguugaugaaacgccggacauacaauuuacagccacagcgaggcuauuuauuccggaaaauaauuuugacacauggacagguauauuucuuucaacaaacaucaauagaaugaaauguccaguccacaucacaugcuucaccuucauuuuuguagaagaucauacccccgaaaaacauuaaggaacuucgauuccuccacaaaaaaugucuccaaauaugcuaaccuuauuuuucucucuacagcaaggcgaguguggaaggacuacuucccugcuguugacgccauugucuuuuuaguagaugccaaugauaggaacagauucaaagagaguaaacaagaauuggauucgcuacucacagacgagacucuuucgaauuguccgguucuuauauuagguaacaaaauugauuugccucaagcagcuucggaagacgaacuaagaaauuacuaugccuuguauggccaaacaacuggaaaggugaguguaauuuuauuauuaauguugaguaaggaguuuugcaugagaaauauuuuauuucgaucauuagguggucauuuuggaacgauucuugguguuaaaaguguauaggagg

SEQ ID NO: 21 - RNA, 373 nt, Artificial Sequence
  Synthetic polynucleotide
aaugaaauguccaguccacaucacaugcuucaccuucauuuuuguagaagaucauacccccgaaaaacauuaaggaacuucgauuccuccacaaaaaaugucuccaaauaugcuaaccuuauuuuucucucuacagcaaggcgaguguggaaggacuacuucccugcuguugacgccauugucuuuuuaguagaugccaaugauaggaacagauucaaagagaguaaacaagaauuggauucgcuacucacagacgagacucuuucgaauuguccgguucuuauauuagguaacaaaauugauuugccucaagcagcuucggaagacgaacuaagaaauuacuaugccuuguauggccaaacaacuggaaagg

SEQ ID NO: 22 - RNA, 1010 nt, Artificial Sequence
  Synthetic polynucleotide
cuacuauuaaaacuauuuuuuuuauauauacuacacgauaaaaaaccaauuauaccgaaaaaaaaaggucacauaugaauugauauagaaaaaccuuguucaugaaagaauaccguguagguacaccuuaacgaauccacucgcacaaaaaaacuucagucgauguacugggccagccaacggaaaccuucuccguaucccugccucuucagaauggagcacaugaagagcucgaggggacguccuggcaaaucggaccuugccaccuuucccuuuccaguuguuuggccauacaaggcauaguaauuucuuaguucgucuuccgaagcugcuugaggcaaaucaauuuuguuaccuaauauaagaaccggacaauucgaaagagucucgucugugaguagcgaauccaauucuuguuuacucucuuugaaucuguuccuaucauuggcaucuacuaaaaagacaauggcgucaacagcagggaaguaguccuuccacacucgccuugcuguagagagaaaaauaagguuagcauauuuggagacauuuuuuguggaggaaucgaaguuccuuaauguuuuucggggguaugaucuucuacaaaaaugaaggugaagcaugugauguggacuggacauuucauuaugaaaaauguuaagagccuuacuaggauguaaagugggaagaugcugugccagccuaucaucuuugagcauaugcaguaaaguuguuuuuccagcauuguccaggccuaggaaaagcagcuugccugauuucuuccauaaucccaaguaaccaaguacaccgguaaaccaaucccagaugaacauuuuuguaauuugaacuaacguuggaaacgaccaauuaucccacaaaaaauauguaaaguugauauucacuacagaaaagucuuuaaaaucgcccaauuauuauuuuaagaguuuuuuaauuugauaguaacaguucuguuacuuaugguugacaguaaaggagaaguuggcagaaugacg

SEQ ID NO: 23 - RNA, 600 nt, Artificial Sequence
  Synthetic polynucleotide
ccuccuauacacuuuuaacaccaagaaucguuccaaaaugaccaccuaaugaucgaaauaaaauauuucucaugcaaaacuccuuacucaacauuaauaauaaaauuacacucaccuuuccaguuguuuggccauacaaggcauaguaauuucuuaguucgucuuccgaagcugcuugaggcaaaucaauuuuguuaccuaauauaagaaccggacaauucgaaagagucucgucugugaguagcgaauccaauucuuguuuacucucuuugaaucuguuccuaucauuggcaucuacuaaaaagacaauggcgucaacagcagggaaguaguccuuccacacucgccuugcuguagagagaaaaauaagguuagcauauuuggagacauuuuuuguggaggaaucgaaguuccuuaauguuuuucggggguaugaucuucuacaaaaaugaaggugaagcaugugauguggacuggacauuucauucuauugauguuuguugaaagaaauauaccuguccaugugucaaaauuauuuuccggaauaaauagccucgcuguggcuguaaauuguauguccggcguuucaucaacaauugc

SEQ ID NO: 24 - RNA, 373 nt, Artificial Sequence
  Synthetic polynucleotide
ccuuuccaguuguuuggccauacaaggcauaguaauuucuuaguucgucuuccgaagcugcuugaggcaaaucaauuuuguuaccuaauauaagaaccggacaauucgaaagagucucgucugugaguagcgaauccaauucuuguuuacucucuuugaaucuguuccuaucauuggcaucuacuaaaaagacaauggcgucaacagcagggaaguaguccuuccacacucgccuugcuguagagagaaaaauaagguuagcauauuuggagacauuuuuuguggaggaaucgaaguuccuuaauguuuuucggggguaugaucuucuacaaaaaugaaggugaagcaugugauguggacuggacauuucauu

(19) United States
(12) Patent Application Publication
(10) Pub. No.: US 2022/0061335 A1
(43) Pub. Date: Mar. 3, 2022

(54) CONTROL OF INSECT INFESTATION

(71) Applicant: GreenLight Biosciences, Inc., Medford, MA (US)

(72) Inventors: Thais Barros Rodrigues, Durham, NC (US); Suresh D. Desai, Apex, NC (US)

(73) Assignee: GreenLight Biosciences, Inc., Medford, MA (US)

(21) Appl. No.: 17/312,740

(22) PCT Filed: Dec. 10, 2019
(86) PCT No.: PCT/US2019/065326

§ 371 Date: Jun. 10, 2021

Related U.S. Application Data
(60) Provisional application No. 62/787,411, filed on Jan. 2, 2019.
(60) Provisional application No. 62/781,525, filed on Dec. 18, 2018.
(60) Provisional application No. 62/777,980, filed on Dec. 11, 2018.

(51) Int. Cl. A01N 63/60; C12N 15/113

(52) U.S. Cl. CPC A01N 63/60; C12N 2310/14; C12N 15/1137

(57) ABSTRACT

Provided herein are methods for using RNAi molecules targeting a vATPase-E, PTSA2, or SAR1 gene for controlling Coleopteran insects, methods for producing RNAi molecules targeting vATPase-E, PTSA2, or SAR1, and compositions comprising RNAi molecules targeting vATPase-E, PTSA2, or SAR1.

### RELATED APPLICATIONS

This application claims the benefit under 35 U.S.C. § 119(e) of U.S. provisional application No. 62/787,411, filed Jan. 2, 2019, U.S. provisional application No. 62/781,525, filed Dec. 18, 2018, and U.S. provisional application No. 62/777,980 filed Dec. 11, 2018, each of which is incorporated by reference herein in its entirety.

### BACKGROUND

Crops are often the target of insect attacks. Globally, farmers lose 30 to 40 percent of their crops due to pests and diseases, according to the UN Food and Agricultural Organization. Crop maintenance and crop health are essential for yield and quality of produce, which ultimately require long-term strategies for the minimization of pest and disease occurrence. The annual costs of controlling crop pests (e.g., Coleoptera) are estimated to be in the tens of millions of dollars, with projected annual costs of crop loss reaching billions of dollars if left uncontrolled.

While chemical pesticides have been one solution for eradicating pest infestations, alternative, more environmentally safe, solutions are needed. Chemical pesticides are harmful to the environment and may lack specificity or selectivity which ultimately results in non-target effects. Additionally, given the slow metabolism of chemical pesticides and the likelihood of chemical pesticides to accumulate, resistance is likely to occur. Thus, there has been a long-felt need for more environmentally friendly methods for controlling or eradicating insect infestations which are more selective, environmentally safe, and biodegradable.

### SUMMARY

The present disclosure provides, in some aspects, compositions, genetic constructs, and methods for controlling Colorado potato beetle infestation that cause damage to crop plants. For example, aspects of the present disclosure provide compositions that include interfering RNA molecules (e.g., double-stranded RNA) for controlling crop infestation by these pests. Aspects of the present disclosure further provide methods for controlling a pest including, but not limited to, killing the pest, inhibiting the growth and development of the pest, altering fertility or growth of the pest such that the pest provides less damage to a crop plant, decreasing the number of offspring produced by a pest, producing less fit pests, reducing insect infestation populations, producing pests more susceptible to predator attack, or deterring the pests from eating a crop plant. To reduce dependence on broad-spectrum chemical insecticides and their related problems, reduced-risk pesticides are required. A new technology that offers the promise of a reduced risk approach to insect pest control is RNA interference (RNAi). In some embodiments, the present disclosure provides RNAi-based technologies that can mitigate insect (e.g., Colorado potato beetle) damage by delivering ribonucleic acid (RNA) interference (RNAi) molecules that target (e.g., bind to) and interfere with the messenger RNA (mRNA) of an insect (e.g., Colorado potato beetle) Vacuolar ATPase-E (vATPase-E) (e.g., vATPase-E1 or vATPase-E2) gene, a Proteasome Alpha type-2 (PTSA2) gene, or a Secretion Associated Ras Related GTPase 1 (SAR1) gene.

v-ATPase-E is responsible for acidifying a variety of intracellular compartments in eukaryotic cells. vATPase-E1 encodes a component of v-ATPase-E, a multi-subunit enzyme that mediates acidification of eukaryotic intracellular organelles. vATPase-E2 is essential for energy coupling involved in acidification of acrosome. v-ATPase-E dependent organelle acidification is necessary for such intracellular processes as protein sorting, zymogen activation, receptor-mediated endocytosis, and synaptic vesicle proton gradient generation.

PTSA2 is a multi-catalytic proteinase complex which is characterized by its ability to cleave peptides with Arg, Phe, Tyr, Leu, and Glu adjacent to the leaving group at neutral or slightly basic pH. PTSA2 has ATP-dependent proteolytic activity (see UniProtKB-P40301 (PSA2_DROME)). Proteasome-dependent degradation serves an essential role in the removal of a wide variety of key nuclear and cytosolic proteins (Rock K L et al., 1994, Cell 78:761-771; Ulrich H D et al., 2002, Curr. Top. Microbiol. Immunol. 268:137-174; von Arnim A G et al., 2001, Sci. STKE 2001:PE2; and Zwickl P et al., 2001, Adv. Protein Chem. 59:187-222). The pathway also carries out a housekeeping function by clearing cells from potentially harmful abnormal proteins that arise as the result of mutations, translational errors, misfolding, or postsynthetic damage and functions in the cytoplasm as a part of the protein quality control system for the endoplasmic reticulum (Kostova A et al., 2003, EMBO J. 22:2309-2317). See, e.g., Lundgren J et al., 2005, Mol Cell Biol. 25(11):4662-4675, incorporated herein by reference.

SAR1 is primarily involved in transport from the endoplasmic reticulum to the Golgi apparatus and in the selection of the protein cargo and the assembly of the COPII coat complex. SAR1 is activated by the guanine nucleotide exchange factor PREB. SAR1 is also known to interact selectively and non-covalently with a scaffold protein. Scaffold proteins are crucial regulators of many key signaling pathways. Although not strictly defined in function, they are known to interact and/or bind with multiple members of a signaling pathway, tethering them into complexes.

Laboratory studies have confirmed that oral delivery of RNA molecules whose mode of action is through the RNAi process (e.g., double-stranded RNA (dsRNA)) are effective for many insect species and hence, topical dsRNA is considered a suitable form of delivery. However, spray-on dsRNA insect pest control technology does not exist today. The cost of production of dsRNA at a relatively low price is a major challenge for the Ag-Bio industry. For agricultural pests, transgenic plants that can express insecticidal dsRNA may protect the plants from insect herbivory. However, not all countries are receptive to genetically-modified crops, and spray-on application of dsRNA is being considered as an alternative delivery method of protection.

To identify targets for RNAi knockdown, whole genome information was used to identify the appropriate gene sequence for vATPase-E, PTSA2, or SAR1 in the target species (e.g., Leptinotarsa decemlineata), which when silenced selectively, controls these key pests, without adversely affecting non-target species in the potato agriculture ecosystem. The dsRNA was identified by searching comprehensive sequence databases for Tribolium and Drosophila genomes (e.g., Flybase, SnapDragon, Beetlebase, etc.).

In some embodiments, the RNAi molecules comprise single-stranded RNA (ssRNA), and in some embodiments, the RNAi molecules comprise double-stranded RNA (dsRNA) or partially dsRNA. In still other embodiments, the RNAi molecules may be single-stranded RNA molecules with secondary structure containing significant double-stranded character, such as, but not limited to, hairpin RNA. The present disclosure provides RNA, for example single stranded RNA (ssRNA), small interfering RNA (siRNA), micro RNA (miRNA), messenger RNA (mRNA), short hairpin RNA (shRNA) or double stranded RNA (dsRNA) for targeting vATPase-E, PTSA2, or SAR1 mRNA.

An RNAi molecule that targets any one of vATPase-E, PTSA2, or SAR1, in some embodiments, is effective for reducing vATPase-E, PTSA2, or SAR1 expression in an insect, stunting of larvae, inhibiting growth, reproduction (e.g., fertility and/or fecundity) and/or repair of the insect, killing of the larvae or the insect, and decreasing feeding of the insect. Accordingly, one aspect of the present disclosure provides a method for controlling an insect comprising delivering (e.g., contacting) an effective amount of a vATPase-E-targeting RNA, PTSA2-targeting RNA, or SAR1-targeting RNA with a plant and/or an insect. vATPase-E-targeting RNA, PTSA2-targeting RNA, or SAR1-targeting RNA are particularly useful for controlling a Coleopteran insect (e.g., Colorado potato beetle), thereby reducing and/or preventing infestation of certain plants (e.g., a potato) that are a major food source for humans.

Some aspects of the present disclosure also provide cell-free methods of producing vATPase-E-targeting RNA, PTSA2-targeting RNA, or SAR1-targeting RNA, the method comprising: (a) incubating in a reaction mixture cellular RNA, and a ribonuclease under conditions appropriate for the production of 5′ nucleoside monophosphates (5′ NMPs); (b) eliminating the ribonuclease; and (c) incubating the reaction mixture, or in a second reaction mixture, the 5′ NMPs, a polyphosphate kinase, a polyphosphate, a polymerase, and a DNA (also referred to a DNA template) under conditions appropriate for the production of the vATPase-E-targeting RNA, PTSA2-targeting RNA, or SAR1-targeting RNA from the DNA.

Also provided herein are compositions comprising a vATPase-E-targeting RNA, PTSA2-targeting RNA, or SAR1-targeting RNA. In some embodiments, the composition comprising a vATPase-E-targeting RNA, PTSA2-targeting RNA, or SAR1-targeting RNA further comprises an additive, for example, a chemical, a pesticide, a surfactant, a biological, or other non-pesticidal ingredient. In some embodiments, vATPase-E-targeting RNA, PTSA2-targeting RNA, or SAR1-targeting RNA is provided in an expression vector. In some embodiments, a vATPase-E-targeting RNA, PTSA2-targeting RNA, or SAR1-targeting RNA is provided in a plant or a plant cell.

It should be understood that an “RNAi molecule targeting vATPase-E” encompasses “RNAi molecules targeting mRNA encoded by vATPase-E”; that an “RNAi molecule targeting PTSA2” encompasses “RNAi molecules targeting mRNA encoded by PTSA2”; and that “RNAi molecule targeting SAR1” encompasses “RNAi molecules targeting mRNA encoded by SAR1.” An RNAi molecule is considered to target a gene of interest if the RNAi molecule binds to (e.g., transiently binds to) and inhibits (reduces or blocks) translation of the mRNA, e.g., due to the mRNA being degraded. In some embodiments, if there are epigenetic changes, an RNAi molecule may inhibit expression of the mRNA encoded by the gene of interest. It should also be understood that in some embodiments, the polynucleotide is a double-stranded RNA (e.g., dsRNA) that inhibits expression of a coding region of the gene (e.g., vATPase-E, PTSA2, or SAR1). In other embodiments, the polynucleotide is a DNA sequence that encodes a dsRNA. In yet other embodiments, the polynucleotide is an antisense RNA. It should be understood that the sequences disclosed herein as DNA sequences can be converted from a DNA sequence to an RNA sequence by replacing each thymidine with a uracil.

### BRIEF DESCRIPTION OF THE DRAWINGS

FIGS. 1A-1B include graphs showing the percent mortality of Colorado potato beetles (CPBs) (FIG. 1A) and percent leaf disc consumption by CPBs (FIG. 1B) following a nine-day exposure of the CPBs to either a vATPase-E1 RNAi (GS 58) composition of the present disclosure or to a control RNAi (GS 4) composition (10 μg/cm² concentration of RNAi).

FIGS. 2A-2B include graphs showing the percent mortality of Colorado potato beetles (CPBs) (FIG. 2A) and percent leaf disc consumption by CPBs (FIG. 2B) following a nine-day exposure of the CPBs to either a vATPase-E2 RNAi (GS 59) composition of the present disclosure or to a control RNAi (GS 4) composition (10 μg/cm² concentration of RNAi).

FIGS. 3A-3B include graphs showing the percent mortality of Colorado potato beetles (CPBs) (FIG. 3A) and percent leaf disc consumption by CPBs (FIG. 3B) following a three-day dose-trial time course in CPBs exposed to either a vATPase-E1 RNAi (GS 58) composition of the present disclosure (at 1.0 μg/cm², 0.1 μg/cm², 0.01 μg/cm², or 0.001 μg/cm²), a control RNAi composition (GS 4 or GS 1 at 1.0 μg/cm²). Untreated CPBs are also included as control experiments.

FIGS. 4A-4B include graphs showing the percent mortality of Colorado potato beetles (CPBs) (FIG. 4A) and percent leaf disc consumption by CPBs (FIG. 4B) following an eight-day exposure of the CPBs to a PTSA2 RNAi (GS 51) composition of the present disclosure or to a control RNAi (GS 4) composition (10 μg/cm² concentration of RNAi).

FIGS. 5-5B include graphs showing the percent mortality of Colorado potato beetles (CPBs) (FIG. 5A) and percent leaf disc consumption by CPBs (FIG. 5B) following an eight-day exposure of the CPBs to either a SAR1 (GS 48) composition of the present disclosure or to a control RNAi (GS 4) composition (10 μg/cm² concentration of RNAi).

### DETAILED DESCRIPTION

According to some aspects of the present disclosure, RNAi molecules (e.g., dsRNAs) targeting vATPase-E, PTSA2, or SAR1 are effective at interfering with the mRNA encoded by a vATPase-E, PTSA2, or SAR1 gene in insect (e.g. Coleopteran) cells, thereby reducing or eliminating translation of the mRNA (e.g., into its corresponding protein). Accordingly, in some aspects, the present disclosure provides compositions and methods for controlling insect (e.g. Coleopteran) infestations by contacting any portion of a plant (e.g., roots, tubers, stem, branches, leaves, flower, etc.), ground (e.g., soil, dirt, grass, etc.), insect (e.g., Coleopteran) and/or diet (e.g., food and/or water ingested by) of the insect with an RNAi molecule as provided herein. Also provided herein are cell-free methods of synthesizing RNAi molecules that target vATPase-E, PTSA2, or SAR1 gene products (mRNA).

An insect, as used herein, refers to an insect in any stage of development. In some embodiments, the insect is an insect egg. In some embodiments, the insect is an insect larva. In some embodiments, the insect is an insect pupa. In some embodiments, the insect is an adult insect.

A Coleopteran insect may be any Coleopteran insect of order Coleoptera. Examples of insects of the order Coleoptera include, but are not limited to, Chrysomelidae (leaf beetle, broad-shouldered leaf beetle, alligator weed flea beetle), Curculionidae (snout beetle), Meloidae (blister beetle), Tenebrionidae (darkling beetle), Scarabaeidae (scarab beetle), Cerambycidae (Japanese pine sawyer), Curculionidae (Chinese white pine beetle), Nitidulidae (small hive beetle), Cerambycidae (mulberry longhorn beetle), Phyllotreta (flea beetle), Diabrotica (corn rootworm) Chrysomela (cottonwood leaf beetle), Hypothenemus (coffee berry borer), Sitophilus (maize weevil), Epitrix (tobacco flea beetle), E. cucumeris (potato flea beetle), P. pusilla (western black flea beetle); Anthonomus (pepper weevil), Hemicrepidus (wireworms), Melanotus (wireworm), Ceutorhynchus (cabbage seedpod weevil), Aeolus (wireworm), Horistonotus (sand wireworm), Sphenophorus (maize billbug), S. zea (timothy billbug), S. parvulus (bluegrass billbug), S. callosus (southern corn billbug); Phyllophaga (white grubs), Chaetocnema (corn flea beetle), Popillia (Japanese beetle), Epilachna (Mexican bean beetle), Cerotoma (bean leaf beetle), Epicauta (blister beetle), and any combination thereof.

Further, the Coleopteran insect may be any species of Leptinotarsa. Leptinotarsa species include, but are not limited to, Leptinotarsa decemlineata (Colorado potato beetle), Leptinotarsa juncta (False potato Beetle), Leptinotarsa behrensi, Leptinotarsa collinsi, Leptinotarsa defecta, Leptinotarsa haldemani (Haldeman's green potato beetle), Leptinotarsa heydeni, Leptinotarsa juncta (false potato beetle), Leptinotarsa lineolata (burrobrush leaf beetle), Leptinotarsa peninsularis, Leptinotarsa rubiginosa, Leptinotarsa texana, Leptinotarsa tlascalana, Leptinotarsa tumamoca, and Leptinotarsa typographica.

### RNAi Molecules

RNAi molecules targeting vATPase-E, PTSA2, or SAR1 have been identified through examination of vATPase-E, PTSA2, or SAR1 mRNA, in vitro and in vivo (e.g., plant) testing. Such RNAi molecules targeting vATPase-E, PTSA2, or SAR1 are useful for controlling Coleopteran insects (e.g., Colorado potato beetles), for example, by inhibiting or reducing expression of vATPase-E, PTSA2, or SAR1, and consequently, by increasing insect mortality, as well as decreasing growth, reproduction (e.g., fertility and/or fecundity), and/or feeding (e.g., eating and/or drinking) of Coleopteran insects.

Expression of a gene in a cell (e.g., insect cell), for example, is considered to be inhibited or reduced through contact with an RNAi molecule if the level of mRNA and/or protein encoded by the gene is reduced in the cell by at least 10% relative to a control cell that has not been contacted with the RNAi molecule. For example, delivering to a cell (e.g., contacting a cell) with an RNAi molecule (e.g., dsRNA) targeting vATPase-E, PTSA2, or SAR1 may result in a reduction (e.g., by at least 10%) in the amount of RNA transcript and/or protein (e.g., encoded by the vATPase-E, PTSA2, or SAR1 gene) compared to a cell that is not contacted with RNAi molecular targeting vATPase-E, PTSA2, or SAR1.

In some embodiments, RNAi molecules of the present disclosure specifically inhibit expression of a vATPase-E, PTSA2, or SAR1 gene without biologically relevant or biologically significant off-target effects (no relevant or significant change in the expression of non-target genes). In some embodiments, an RNAi molecule specifically inhibits (reduces or blocks) translation of a vATPase-E, PTSA2, or SAR1 protein by specifically inhibiting expression of (e.g., degrading) a vATPase-E, PTSA2, or SAR1 mRNA (e.g., mRNA of any one of SEQ ID NOS: 2, 10, 11, or 19) that encodes the vATPase-E, PTSA2, or SAR1 protein. Specific inhibition of a vATPase-E, PTSA2, or SAR1 gene includes a measurable reduction in vATPase-E, PTSA2, or SAR1 gene expression (e.g., vATPase-E, PTSA2, or SAR1 mRNA expression, and/or vATPase-E, PTSA2, or SAR1 protein expression) or a complete lack of detectable gene expression (e.g., vATPase-E, PTSA2, or SAR1 mRNA expression, and/or vATPase-E, PTSA2, or SAR1 protein expression).

In some embodiments, an RNAi molecule specifically inhibits the expression of a vATPase-E, PTSA2, or SAR1 protein by specifically inhibiting an mRNA that encodes a vATPase-E, PTSA2, or SAR1 protein (e.g., mRNA of SEQ ID NOS: 2, 10, 11, or 19). Specific inhibition of a vATPase-E, PTSA2, or SAR1 gene involves a measurable reduction in vATPase-E, PTSA2, or SAR1 gene expression (e.g., vATPase-E, PTSA2, or SAR1 mRNA expression, and/or vATPase-E, PTSA2, or SAR1 protein expression) or a complete lack of detectable gene expression (e.g., vATPase-E, PTSA2, or SAR1 mRNA expression, and/or vATPase-E, PTSA2, or SAR1 protein expression).

RNAi molecules targeting vATPase-E, PTSA2, or SAR1 provided herein, in some embodiments, are designed to have complementarity to vATPase-E, PTSA2, or SAR1 mRNA of a Coleopteran insect, e.g., a Colorado potato beetle. An example of a DNA sequence encoding Colorado potato beetle vATPase is provided in the sequence of SEQ ID NO: 1. An example of a DNA sequence encoding Colorado potato beetle PTSA2 is provided in the sequence of SEQ ID NO: 8 or 9. An example of a DNA sequence encoding Colorado potato beetle SAR1 is provided in the sequence of SEQ ID NO: 18. An example of an mRNA sequence encoding Colorado potato beetle vATPase-E is provided in the sequence of SEQ ID NO: 2. An example of an mRNA sequence encoding Colorado potato beetle PTSA2 is provided in the sequence of SEQ ID NO: 10 or 11. An example of an mRNA sequence encoding Colorado potato beetle SAR1 is provided in the sequence of SEQ ID NO: 19. Examples of Colorado potato beetle vATPase-E mRNA sequences targeted by an RNAi molecule of the present disclosure encoding are provided in the sequences of SEQ ID NOS: 2-4. Examples of RNA molecules targeting vATPase-E are provided in the sequences of SEQ ID NOS: 5-7. Examples of Colorado potato beetle PTSA2 mRNA sequences targeted by an RNAi molecule of the present disclosure encoding are provided in the sequences of SEQ ID NOS: 10-13. Examples of RNA molecules targeting PTSA2 are provided in the sequences of SEQ ID NOS: 14-17. Examples of Colorado potato beetle SAR1 mRNA sequences targeted by an RNAi molecule of the present disclosure encoding are provided in the sequences of SEQ ID NOS: 19-21. Examples of RNA molecules targeting SAR1 are provided in the sequences of SEQ ID NOS: 22-24.

In some embodiments, the RNAi molecule targeting vATPase-E, PTSA2, or SAR1 provided herein is designed to have complementarity to vATPase-E, PTSA2, or SAR1 mRNA of a Coleopteran insect, e.g., a Chrysomelidae (a leaf beetle), a Curculionidae (a snout beetle), a Meloidae (a blister beetle), Tenebrionidae (a darkling beetle), a Scarabaeidae (a scarab beetle), a Cerambycidae (a Japanese pine sawyer), a Curculionidae (a Chinese white pine beetle), a Nitidulidae (a small hive beetle), a Chrysomelidae (a broad-shouldered leaf beetle), a Cerambycidae (a mulberry longhorn beetle), C. scripta (cottonwood leaf beetle), H. hampei (coffee berry borer), S. Zeamais (maize weevil), F. hirtipennis (tobacco flea beetle), F. cucumeris (potato flea beetle), P. cruciferae (crucifer flea beetle) and P. pusilla (western black flea beetle), A. eugenii (pepper weevil), H. memnonius (wireworms), M. communis (wireworm), C. assimilis (cabbage seedpod weevil), P. striolata (striped flea beetle), A. mellillus (wireworm), A. mancus (wheat wireworm), H. uhlerii (sand wireworm), S. maidis (maize billbug), S. zeae (timothy billbug), S. parvulus (bluegrass billbug), and S. callosus (southern corn billbug), Phyllophaga spp. (White grubs), C. pulicaria (corn flea beetle), P. japonica (Japanese beetle), F. varivestis (Mexican bean beetle), C. trifurcate (Bean leaf beetle), F. pestifera and F. lemniscata (Blister beetles), Oulema melanapus (Cereal leaf beetle), Hypera postica (Alfalfa weevil), Dendroctonus (Mountain Pine beetle), Agrilus (Emerald Ash Borer), Hylurgopinus (native elm bark beetle), Scolytus (European elm bark beetle) and/or a Chrysomelidae (an alligator weed flea beetle).

In some embodiments, the RNAi molecule targeting vATPase-E, PTSA2, or SAR1 provided herein is designed to have complementarity to vATPase-E, PTSA2, or SAR1 mRNA of a Leptinotarsa insect, e.g., a Leptinotarsa decemlineata (a Colorado potato beetle), a Leptinotarsa behrensi, a Leptinotarsa collinsi, a Leptinotarsa defecta, a Leptinotarsa haldemani (a Haldeman's green potato beetle), a Leptinotarsa heydeni, a Leptinotarsa juncta (a false potato beetle), a Leptinotarsa lineolata (a burrobrush leaf beetle), a Leptinotarsa peninsularis, a Leptinotarsa rubiginosa, a Leptinotarsa texana, a Leptinotarsa tlascalana, a Leptinotarsa tumamoca, and/or a Leptinotarsa typographica.

A double-stranded RNA (dsRNA) of the present disclosure, in some embodiments, comprises a first strand that binds to (e.g., is at least partially complementary to or is wholly complementary to) a messenger RNA (mRNA) encoded by a Coleoptera vATPase-E, PTSA2, or SAR1 gene, and a second strand that is complementary to the first strand.

dsRNA may comprise RNA strands that are the same length or different lengths. In some embodiments, a dsRNA comprises a first strand (e.g., an antisense strand) that is the same length as a second strand (e.g., a sense strand). In some embodiments, a dsRNA comprises a first strand (e.g., an antisense strand) that is a different length than a second strand (e.g., a sense strand). A first strand may be about 1%, 2%, 3%, 4%, 5%, 10%, 15%, 20%, or more than 20% longer than a second strand. A first strand may be 1-5, 2-5, 2-10, 5-10, 5-15, 10-20, 15-20, or more than 20 nucleotides longer than a second strand.

dsRNA molecules can also be assembled from a single oligonucleotide in a stem-loop structure, wherein self-complementary sense and antisense regions of the RNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active RNAi molecule capable of mediating RNAi. An RNAi molecule may comprise a 3′ overhang at one end of the molecule; the other end may be blunt-ended or have also an overhang (5′ or 3′). When the RNAi molecule comprises an overhang at both ends of the molecule, the length of the overhangs may be the same or different.

A single-stranded RNA of the present disclosure, in some embodiments, comprises a strand that binds to a mRNA encoded by a Coleoptera vATPase-E, PTSA2, or SAR1 gene.

RNAi molecules targeting vATPase-E, PTSA2, or SAR1 as provided herein may vary in length. It should be understood that, in some embodiments, while a long RNA (e.g., dsRNA or ssRNA) molecule is applied (e.g., to a plant) as the insecticide, after entering cells the dsRNA is cleaved by the Dicer enzyme into shorter double-stranded RNA fragments having a length of, for example, 15 to 25 nucleotides. Thus, RNAi molecules of the present disclosure may be delivered as 15 to 25 nucleotide fragments, for example, or they may be delivered as longer double-stranded nucleic acids (e.g., at least 100 nucleotides).

Thus, in some embodiments, RNAi molecules targeting vATPase-E, PTSA2, or SAR1 comprise 15-1010 nucleotides (ssRNA) or nucleotide base pairs (dsRNA). For example, an RNAi molecule of the present disclosure may comprise 15-1000, 15-950, 15-900, 15-850, 15-800, 15-750, 15-700, 15-650, 15-600, 15-500, 15-450, 15-400, 15-350, 15-300, 15-250, 15-200, 15-150, 15-100, 15-50, 16-1000, 16-950, 16-900, 16-850, 16-800, 16-750, 16-700, 16-650, 16-600, 16-500, 16-450, 16-400, 16-350, 16-300, 16-250, 16-200, 16-150, 16-100, 16-50, 17-1000, 17-950, 17-900, 17-850, 17-800, 17-750, 17-700, 17-650, 17-600, 17-500, 17-450, 17-400, 17-350, 17-300, 17-250, 17-200, 17-150, 17-100, 17-50, 18-1000, 18-950, 18-900, 18-850, 18-800, 18-750, 18-700, 18-650, 18-600, 18-500, 18-450, 18-400, 18-350, 18-300, 18-250, 18-200, 18-180, 18-100, 18-50, 19-1000, 19-950, 19-900, 19-850, 19-800, 19-750, 19-700, 19-650, 19-600, 19-500, 19-450, 19-400, 19-350, 19-300, 19-250, 19-200, 19-190, 19-100, 19-50, 20-1000, 20-950, 20-900, 20-850, 20-800, 20-750, 20-700, 20-650, 20-600, 20-500, 20-450, 20-400, 20-350, 20-300, 20-250, 20-200, 20-200, 20-100, 20-50, 21-1000, 21-950, 21-900, 21-850, 21-800, 21-750, 21-700, 21-650, 21-600, 21-500, 21-450, 21-400, 21-350, 21-300, 21-250, 21-210, 21-210, 21-100, 21-50, 22-1000, 22-950, 22-900, 22-850, 22-800, 22-750, 22-700, 22-650, 22-600, 22-500, 22-450, 22-400, 22-350, 22-300, 22-250, 22-220, 22-220, 22-100, 22-50, 23-1000, 23-950, 23-900, 23-850, 23-800, 23-750, 23-700, 23-650, 23-600, 23-500, 23-450, 23-400, 23-350, 23-300, 23-250, 23-230, 23-230, 23-100, 23-50, 24-1000, 24-950, 24-900, 24-850, 24-800, 24-750, 24-700, 24-650, 24-600, 24-500, 24-450, 24-400, 24-350, 24-300, 24-250, 24-240, 24-240, 24-100, 24-50, 25-1000, 25-950, 25-900, 25-850, 25-800, 25-750, 25-700, 25-650, 25-600, 25-500, 25-450, 25-400, 25-350, 25-300, 25-250, 25-250, 25-250, 25-100, or 25-50 nucleotides or nucleotide base pairs. In some embodiments, RNAi molecules targeting vATPase-E, PTSA2, or SAR1 comprise or consist of at least 15, at least 16, at least 17, at least 18, at least 19, at least 20, at least 25, at least 50, at least 200, at least 300, at least 400, at least 500, at least 600, at least 700, at least 800, at least 900, or at least 1000 nucleotides or nucleotide base pairs.

In some embodiments, an RNAi molecule targeting vATPase-E, PTSA2, or SAR1 comprises or consists of a sequence that is complementary to an mRNA or a segment of an mRNA encoded by a Coleoptera vATPase-E, PTSA2, or SAR1 gene.

In some embodiments, an RNAi molecule targeting vATPase-E comprises or consists of a sequence that is complementary to an mRNA or a segment of an mRNA encoded by a DNA sequence of SEQ ID NO: 1. In some embodiments, an RNAi molecule targeting PTSA2 comprises or consists of a sequence that is complementary to an mRNA or a segment of an mRNA encoded by a DNA sequence of SEQ ID NO: 8 or 9. In some embodiments, an RNAi molecule targeting SAR1 comprises or consists of a sequence that is complementary to an mRNA or a segment of an mRNA encoded by a DNA sequence of SEQ ID NO: 18.

In some embodiments, an RNAi molecule targeting vATPase-E, PTSA2, or SAR1 comprises or consists of a sequence that is complementary to an mRNA encoded by a region or segment of a Coleoptera vATPase-E, PTSA2, or SAR1 DNA. In some embodiments, an RNAi molecule targets an mRNA encoded by a region of a Coleoptera vATPase-E, PTSA2, or SAR1 DNA that may comprise or consist of any sequence encompassed by nucleotides 1 to 500, nucleotides 10 to 500, nucleotides 25 to 500, nucleotides 50 to 500, nucleotides 100 to 500, nucleotides 150 to 500, nucleotides 200 to 500, nucleotides 250 to 500, nucleotides 300 to 500, nucleotides 350 to 500, nucleotides 400 to 500, or nucleotides 450 to 500 of the vATPase-E, PTSA2, or SAR1 DNA. In some embodiments, an RNAi molecule targets an mRNA encoded by a region of a Coleoptera vATPase-E, PTSA2, or SAR1 DNA that may comprise or consist of any sequence encompassed by nucleotides 200 to 950, nucleotides 250 to 950, nucleotides 300 to 950, nucleotides 350 to 950, nucleotides 400 to 950, nucleotides 450 to 950, nucleotides 500 to 950, nucleotides 550 to 950, nucleotides 200 to 700, nucleotides 250 to 700, nucleotides 300 to 700, nucleotides 350 to 700, nucleotides 400 to 700, nucleotides 450 to 700, nucleotides 500 to 700, nucleotides 550 to 700, nucleotides 600 to 700, or nucleotides 650 to 700 of the vATPase-E, PTSA2, or SAR1 DNA. In some embodiments, an RNAi molecule targets an mRNA encoded by a region or segment of a Coleoptera vATPase-E, PTSA2, or SAR1 DNA that may comprise or consist of any sequence encompassed by nucleotides 400 to 1010, nucleotides 4500 to 1010, nucleotides 500 to 1010, nucleotides 550 to 1010, nucleotides 600 to 1010, nucleotides 650 to 1010, nucleotides 700 to 1010, nucleotides 750 to 1010, nucleotides 800 to 1010, nucleotides 850 to 1010, nucleotides 900 to 1010, or nucleotides 950 to 1010 of the vATPase-E, PTSA2, or SAR1 DNA.

It should be understood that the term ‘gene’ encompasses coding and non-coding nucleic acid. Thus, in some embodiments, a vATPase-E, PTSA2, or SAR1 gene encodes an mRNA that comprises a 5′ untranslated region, an open reading frame, and a 3′ untranslated region. Thus, an RNAi molecule herein, in some embodiments, binds to a 5′ untranslated region, an open reading frame, and/or a 3′ untranslated region of an mRNA.

In some embodiments, an RNAi molecule targeting vATPase-E comprises or consists of an RNA sequence of any one of SEQ ID NOS: 5-7. In some embodiments, an RNAi molecule targeting PTSA2 comprises or consists of an RNA sequence of SEQ ID NOS: 14-17. In some embodiments, an RNAi molecule targeting SAR1 comprises or consists of an RNA sequence of SEQ ID NOS: 22-24.

In some embodiments, an RNAi molecule targeting vATPase-E comprises or consists of a sequence that is complementary to a RNA sequence of any one of SEQ ID NOS: 2-4. In some embodiments, an RNAi molecule targeting PTSA2 comprises or consists of a sequence that is complementary to a RNA sequence of any one of SEQ ID NOS: 10-13. In some embodiments, an RNAi molecule targeting SAR1 comprises or consists of a sequence that is complementary to a RNA sequence of any one of SEQ ID NOS: 19-21.

In some embodiments, RNAi molecules targeting vATPase-E, PTSA2, or SAR1 comprise or consist of a (at least one) contiguous sequence that has 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a Coleoptera vATPase-E, PTSA2, or SAR1 gene, respectively.

In some embodiments, the vATPase gene comprises a DNA sequence of SEQ ID NO: 1. In some embodiments, RNAi molecules targeting vATPase comprise or consist of a (at least one) contiguous sequence that has 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a DNA sequence of SEQ ID NO: 1.

In some embodiments, the PTSA2 gene comprises a DNA sequence of SEQ ID NO: 8 or 9. In some embodiments, RNAi molecules targeting PTSA2 comprise or consist of a (at least one) contiguous sequence that has 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a DNA sequence of SEQ ID NO: 8 or 9.

In some embodiments, the SAR1 gene comprises a DNA sequence of SEQ ID NO: 18. In some embodiments, RNAi molecules targeting SAR1 comprise or consist of a (at least one) contiguous sequence that has 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a DNA sequence of SEQ ID NO: 18.

In some embodiments, RNAi molecules targeting vATPase-E, PTSA2, or SAR1 comprise or consist of a (at least one) contiguous sequence that is 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence encoded by a Coleoptera vATPase-E, PTSA2, or SAR1 gene.

In some embodiments, the vATPase-E gene comprises a DNA sequence of SEQ ID NO: 1. In some embodiments, RNAi molecules targeting vATPase-E comprise or consist of a (at least one) contiguous sequence that is 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence encoded by a DNA sequence of SEQ ID NO: 1.

In some embodiments, the PTSA2 gene comprises a DNA sequence of SEQ ID NO: 8 or 9. In some embodiments, RNAi molecules targeting PTSA2 comprise or consist of a (at least one) contiguous sequence that is 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence encoded by a DNA sequence of SEQ ID NO: 8 or 9.

In some embodiments, the SAR1 gene comprises a DNA sequence of SEQ ID NO: 18. In some embodiments, RNAi molecules targeting SAR1 comprise or consist of a (at least one) contiguous sequence that is 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence encoded by a DNA sequence of SEQ ID NO: 18.

In some embodiments, RNAi molecules targeting vATPase-E comprise or consist of at least 15, at least 16, at least 17, at least 18, at least 19, at least 20, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 2-4.

In some embodiments, RNAi molecules targeting PTSA2 comprise or consist of at least 15, at least 16, at least 17, at least 18, at least 19, at least 20, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 10-13.

In some embodiments, RNAi molecules targeting SAR1 comprise or consist of at least 15, at least 16, at least 17, at least 18, at least 19, at least 20, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 19-21.

In some embodiments, RNAi molecules targeting vATPase-E comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of any one of SEQ ID NOS: 5-7.

In some embodiments, RNAi molecules targeting PTSA2 comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of any one of SEQ ID NOS: 14-17.

In some embodiments, RNAi molecules targeting SAR1 comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of any one of SEQ ID NOS: 22-24.

In some embodiments, RNAi molecules targeting vATPase-E comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 2-4.

In some embodiments, RNAi molecules targeting PTSA2 comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 10-13.

In some embodiments, RNAi molecules targeting SAR1 comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 19-21.

The “percent identity” of two nucleic acid sequences may be determined by any method known in the art. The variants provided herein, in some embodiments, contain randomly placed mutations with the four nucleotides (A, U, G, C) selected at an approximately equal probability for a given mutation. In some embodiments, these mutations might be distributed either over a small region of the sequence, or widely distributed across the length of the sequence. In some embodiments, the percent identity of two nucleic acid sequences is determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul et al. J. Mol. Biol. 215:403-10, 1990. BLAST nucleotide searches can be performed with the NBLAST program, score=100, wordlength-12 to obtain guide sequences homologous to a target nucleic acid. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

The polynucleotides provided herein, such as RNAi molecules targeting vATPase-E, PTSA2, or SAR1, in some embodiments, are designed to have at least one silencing element complementary (e.g., wholly (100%) or partially (less than 100%, e.g., 90% to 99%) complementary) to a segment of a sequence of vATPase-E, PTSA2, or SAR1 mRNA of a Coleopteran insect, e.g., a Colorado potato beetle. In some embodiments, polynucleotides comprise at least one silencing element that is essentially identical or essentially complementary to vATPase-E, PTSA2, or SAR1 mRNA of a Coleopteran insect. In some embodiments, the polynucleotides comprise 2 to 5, to 10, 2 to 20, 2 to 20, 2 to 40, or 2 to 50 silencing elements. In some embodiments, the polynucleotides comprise at least 2, at least 3, at least 4, at least 5, at least 6, at least 7, at least 8, at least 9, at least 10, at least 15, at least 20, at least 25, at least 30, at least 35, at least 40, at least 45 or at least 50 silencing elements.

RNAi molecules targeting vATPase-E, PTSA2, or SAR1 provided herein may be of any form of RNA, including single-stranded RNA (ssRNA) and double-stranded RNA (dsRNA). Non-limiting examples of single-stranded RNA include mRNA, micro RNA (miRNA) (e.g., artificial miRNA (amiRNA)), small interfering RNA (siRNA), piwi-interacting RNA (piRNA), and antisense RNA. Double-stranded RNA includes wholly double-stranded molecules that do not contain a single-stranded region (e.g., a loop or overhang), as well as partially double-stranded molecules that contain a double-stranded region and a single-stranded region (e.g., a loop or overhang). Further, the RNAi molecules may be single-stranded RNA molecules with secondary structure containing significant double-stranded character, such as, but not limited to, hairpin RNA. Thus, RNAi molecules targeting vATPase-E, PTSA2, or SAR1, in some embodiments, may be short hairpin RNA (shRNA).

In some embodiments, RNAi molecules targeting vATPase-E, PTSA2, or SAR1 comprise dsRNA, ssRNA, siRNA, miRNA (e.g., amiRNA), piRNA, mRNA, or shRNA. In some embodiments, RNAi molecules targeting vATPase-E, PTSA2, or SAR1 comprise more than one form of RNA. For example, the RNAi molecules targeting vATPase-E, PTSA2, or SAR1 may comprise ssRNA and dsRNA. In some embodiments, RNAi molecules targeting vATPase-E, PTSA2, or SAR1 comprise a hybrid with RNA and DNA. In some embodiments, RNAi molecules targeting vATPase-E, PTSA2, or SAR1 comprise amiRNAs processed from a long precursor transcript of nonprotein-coding RNA, that is partially self-complementary to mediate silencing of target mRNAs. amiRNAs are designed, in some embodiments, by replacing the mature 21 nucleotide miRNA sequences within pre-miRNA with 21 nucleotide long fragments derived from the target gene (Frontiers in Plant Science, Sebastian et al., 2017). An amiRNA may have a length of, for example, at least 18 to 500 nucleotides, at least 21 to 500 nucleotides, at least 50 to 500 nucleotides, at least 100 to 500 nucleotides, or at least 200 to 500 nucleotides.

RNAi molecules targeting vATPase-E, PTSA2, or SAR1 may be provided as a mixture of RNAi molecules targeting vATPase-E, PTSA2, or SAR1, for example, a mixture of RNAi molecules targeting vATPase-E, PTSA2, or SAR1 having different sequences. Any number of distinct RNAi molecules targeting vATPase-E, PTSA2, or SAR1 may be provided in a mixture of RNAi molecules targeting vATPase-E, PTSA2, or SAR1. In some embodiments, the mixture of RNAi molecules targeting vATPase-E, PTSA2, or SAR1 comprises at least 2, at least 3, at least 4, at least 5, at least 6, at least 7, at least 8, at least 9, or at least 10 distinct (having different sequences/nucleotide compositions) RNAi molecules targeting vATPase-E, PTSA2, or SAR1.

In some embodiment, RNAi molecules targeting vATPase-E, PTSA2, or SAR1 are provided as a mixture of RNAi molecules that are complementary (wholly or partially) to different segments of an mRNA encoded by a vATPase-E, PTSA2, or SAR1 gene. In some embodiment, RNAi molecules targeting vATPase-E are provided as a mixture of RNAi molecules that are complementary (wholly or partially) to different segments of an RNA sequence of SEQ ID NO: 2. In some embodiment, RNAi molecules targeting PTSA2 are provided as a mixture of RNAi molecules that are complementary (wholly or partially) to different segments of an RNA sequence of SEQ ID NO: 10 or 11. In some embodiment, RNAi molecules targeting SAR1 are provided as a mixture of RNAi molecules that are complementary (wholly or partially) to different segments of an RNA sequence of SEQ ID NO: 19. In some embodiments, the mixture of RNAi molecules targeting vATPase-E, PTSA2, or SAR1 comprises at least 2, at least 3, at least 4, at least 5, at least 6, at least 7, at least 8, at least 9, or at least 10 RNAi molecules targeting vATPase-E, PTSA2, or SAR1, respectively. In some embodiments, the mixture of RNAi molecules targeting vATPase-E, PTSA2, or SAR1 comprises 2 to 5, or 2 to 10 RNAi molecules targeting vATPase-E, PTSA2, or SAR1, respectively.

In some embodiments, RNAi molecules targeting vATPase-E, PTSA2, or SAR1 provided herein may have one or more mismatches compared with the corresponding sequence of vATPase-E, PTSA2, or SAR1 mRNA. A region of complementarity on RNAi molecule targeting vATPase-E, PTSA2, or SAR1 may have up to 1, up to 2, up to 3, up to 4, etc. mismatches provided that it maintains the ability to form complementary base pairs with vATPase-E, PTSA2, or SAR1 mRNA under appropriate hybridization conditions. Alternatively, a region of complementarity on RNAi molecules targeting vATPase-E, PTSA2, or SAR1 may have no more than 1, no more than 2, no more than 3, or no more than 4 mismatches provided that it maintains the ability to form complementary base pairs with vATPase-E, PTSA2, or SAR1 mRNA under appropriate hybridization conditions. In some embodiments, if there is more than one mismatch in a region of complementarity, they may be positioned consecutively (e.g., 2, 3, 4, or more in a row), or interspersed throughout the region of complementarity provided that the RNAi molecule targeting vATPase-E, PTSA2, or SAR1 maintains the ability to form complementary base pairs with vATPase-E, PTSA2, or SAR1 mRNA under appropriate hybridization conditions.

RNAi molecules targeting vATPase-E, PTSA2, or SAR1 may be modified in various ways to improve or control specificity, stability, delivery, bioavailability, degradation, resistance to nuclease degradation, base-pairing properties, RNA distribution, and cellular uptake, and other features relevant to its use. See, e.g., Bramsen et al., Nucleic Acids Res., 2009, 37, 2867-2881; Bramsen and Kjems, Frontiers in Genetics, 3 (2012): 1-22. Accordingly, in some embodiments, RNAi molecules targeting vATPase-E, PTSA2, or SAR1 may include one or more (at least one) suitable modifications. In some embodiments, a modified RNAi molecule targeting vATPase-E, PTSA2, or SAR1 has a modification in its base, sugar (e.g., ribose, deoxyribose), or phosphate group.

RNAi molecules targeting vATPase-E, PTSA2, or SAR1 produced by the methods provided herein may be modified as described herein. In some embodiments, RNAi molecules targeting vATPase-E, PTSA2, or SAR1 is produced according to a method described herein and subsequently modified. In some embodiments, RNAi molecules targeting vATPase-E, PTSA2, or SAR1 are produced according to a method described herein using a modified starting material. In some embodiments, the modified starting material is a modified nucleobase. In some embodiments, the modified starting material is a modified nucleoside. In some embodiments, the modified starting material is a modified nucleotide.

In some embodiments, modified RNAi molecules targeting vATPase-E, PTSA2, or SAR1 comprise a backbone modification. In some embodiments, backbone modification results in a longer half-life for the RNA due to reduced degradation (e.g., nuclease-mediated degradation). This in turn results in a longer half-life. Examples of suitable backbone modifications include, but are not limited to, phosphorothioate modifications, phosphorodithioate modifications, p-ethoxy modifications, methylphosphonate modifications, methylphosphorothioate modifications, alkyl- and aryl-phosphates (in which the charged phosphonate oxygen is replaced by an alkyl or aryl group), alkylphosphotriesters (in which the charged oxygen moiety is alkylated), peptide nucleic acid (PNA) backbone modifications, and locked nucleic acid (LNA) backbone modifications. These modifications may be used in combination with each other and/or in combination with phosphodiester backbone linkages.

Alternatively or additionally, RNAi molecules targeting vATPase-E, PTSA2, or SAR1 may comprise other modifications, including modifications at the base or sugar moiety. Examples include RNA having sugars that are covalently attached to low molecular weight organic groups other than a hydroxyl group at the 3′ position and other than a phosphate group at the 5′ position (e.g., a 2′-O-alkylated ribose), or RNA having sugars such as arabinose instead of ribose. RNA also embraces substituted purines and pyrimidines such as C-5 propyne modified bases (Wagner et al., Nature Biotechnology 14:840-844, 1996). Other purines and pyrimidines include, but are not limited to, 5-methylcytosine, 2-aminopurine, 2-amino-6-chloropurine, 2,6-diaminopurine, and hypoxanthine. Other such modifications are well known to those of skill in the art.

RNAi molecules that comprise a nucleotide sequence complementary to all or a segment of the target sequence can be designed and prepared using any suitable methods. In some embodiments, an RNAi molecule may be designed with assistance from comprehensive sequence databases, such as those known for Tribolium and Drosophila genetics (e.g., Flybase, SnapDragon, Beetlebase, etc.). In some embodiments, a sequence database is utilized to determine off-target effects of a designed RNAi molecule (e.g., as in Arziman, Z., Horn, T., & Boutros, M. (2005). E-RNAi: a web application to design optimized RNAi constructs. Nucleic Acids Research, 33 (Web Server issue), W582-W588. doi:10.1093/nar/gki468.)

### Methods of Use

Aspects of the present disclosure, in some embodiments, provide methods for controlling an insect infestation comprising delivering to a plant or insect (e.g., a Coleopteran insect, e.g., a Colorado potato beetle) an effective amount of an RNAi molecule targeting vATPase-E, PTSA2, or SAR1 (or a composition comprising an RNAi molecule targeting vATPase-E, PTSA2, or SAR1). In some embodiments, the method of delivery comprises applying to a surface of a plant or insect, a composition comprising the RNAi molecule. In some embodiments, a composition comprising an RNAi molecule targeting vATPase-E, PTSA2, or SAR1 is a solid or liquid (e.g., solution, suspension, or emulsions). Non limiting examples include emulsifiable concentrates, concentrate solutions, low concentrate solutions, ultra-low volume concentrate solutions, water-soluble concentrate solutions, water-soluble liquid solutions, baits (paste, gel, liquid, solid or injectable), smoke, fog, invert emulsions, flowables, aerosols, homogenous and non-homogenous mixtures, suspensions (water and oil-based), dust, powders (wettable or soluble), granules (water-dispersible or dry flowables), pellets, capsules, fumigants, encapsulated or micro-encapsulation formulations, or any combinations thereof.

In some embodiments, a composition comprising an RNAi molecule targeting vATPase-E, PTSA2, or SAR1 may be applied as a concentrate, spray (after dilution or concentrate), fog, in furrow, seed treatment, seed coating, drench, drip, insect diet, bait, or any other forms suited for applying to a furrow. The RNAi molecule targeting vATPase-E, PTSA2, or SAR1 described herein may be delivered to any portion of a plant, including, but are not limited to, leaf, stem, flower, fruit, shoot, root, seed, tuber, anther, stamen, and/or pollen. In some embodiments, RNAi is delivered mechanically, through high pressure spray or sandblasting. In some embodiments, a composition comprises an RNAi molecules and at least one additive selected from adjuvants, attractants, sterilizing agents, growth-regulating substances, carriers or diluents, stabilizers, and/or pesticidal agent(s) (e.g., insecticides, fungicides, and/or herbicides). Pesticidal agents include, for example, other dsRNA targeting genes distinct from vATPase-E, PTSA2, or SAR1, insecticidal proteins (patatins, plant lectins, phytoecdysteroids, cry proteins, vegetative insecticidal proteins (vip), cytolytic proteins (cyt)), biotin-binding proteins, protease inhibitors, chitinases, organic compounds, or any combination thereof. Non-pesticidal agents may also be used (e.g. adjuvants, such as antifoaming agents, buffers, compatibility agents, drift control additives, emulsifiers, extenders, invert emulsifiers, plant penetrants, safeners, spreaders, stickers, surfactants, thickeners, and wetting agents).

A composition, in some embodiments, include a mixture of an RNAi molecule targeting vATPase-E, PTSA2, or SAR1 and at least one of a variety of agricultural chemicals, insecticides, miticides, fungicides, pesticidal agents and/or biopesticidal (e.g., microbial, plant-incorporated-protectant (PIP), and/or biochemical) agents, such as Spiromesifen, Spirodiclofen, Spirotetramat, Pyridaben, Tebufenpyrad, Tolfenpyrad, Fenpyroximate, Flufenerim, Pyrimidifen, Fenazaquin, Rotenone, Cyenopyrafen, Hydramethylnon, Acequinocyl, Fluacrypyrim, Aluminium phosphide, Calcium phosphide, Phosphine, Zinc phosphide, Cyanide, Diafenthiuron, Azocyclotin, Cyhexatin, Fenbutatin oxide, Propargite, Tetradifon, Bensultap, Thiocyclam, Thiosultap-sodium, Flonicamid, Etoxazole, Clofentezine, Diflovidazin, Hexythiazox, Chlorfluazuron, Bistrifluron, Diflubenzuron, Flucycloxuron, Flufenoxuron, Hexaflumuron, Lufenuron, Novaluron, Noviflumuron, Teflubenzuron, Triflumuron, Buprofezin, Cyromazine, Hydroprene, Kinoprene, Methoprene, Fenoxycarb, Pyriproxyfen, Pymetrozine, Pyrifluquinazon, Chlorfenapyr, Tralopyril, methyl bromide and/or other alkyl halides, Chloropicrin, Sulfuryl fluoride, Benclothiaz, Chinomethionat, Cryolite, Methylneodecanamide, Benzoximate, Cymiazole, Fluensulfone, Azadirachtin, Bifenazate, Amidoflumet, Dicofol, Plifenate, Cyflumetofen, Pyridalyl, Beauveria bassiana GHA, Sulfoxaflor, Spinetoram, Spinosad, Spinosad, Emamectin benzoate, Lepimectin, Milbemectin, Abamectin, Methoxyfenozide, Chromafenozide, Halofenozide, Tebufenozide, Amitraz, Chlorantraniliprole, Cyantraniliprole, Flubendiamide, alpha-endosulfan, Chlordane, Endosulfan, Fipronil, Acetoprole, Ethiprole, Pyrafluprole, Pyriprole, Indoxacarb, Metaflumizone, Acrinathrin, Allethrin, Allethrin-cis-trans, Allethrin-trans, beta-Cyfluthrin, beta-Cypermethrin, Bifenthrin, Bioallethrin, Bioallethrin S-cyclopentenyl, Bioresmethrin, Cycloprothrin, Cyfluthrin, Cyhalothrin, Cypermethrin, Cyphenothrin [(1R)-trans-isomers], Dimefluthrin, Empenthrin [(EZ)-(1R)-isomers], Esfenvalerate, Etofenprox, Fenpropathrin, Fenvalerate, Flucythrinate, Flumethrin, Gamma-cyhalothrin, lambda-Cyhalothrin, Meperfluthrin, Metofluthrin, Permethrin, Phenothrin [(1R)-trans-isomer], Prallethrin, Profluthrin, Protrifenbute, Resmethrin, Silafluofen, tau-Fluvalinate, Tefluthrin, Tetramethrin, Tetramethrin [(1R)-isomers], Tetramethylfluthrin, theta-Cypermethrin, Tralomethrin, Transfluthrin, zeta-Cypermethrin, alpha-Cypermethrin, Deltamethrin, DDT, Methoxychlor, Thiodicarb, Alanycarb, Aldicarb, Bendiocarb, Benfuracarb, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiofanox, Triazamate, Trimethacarb, XMC, Xylylcarb, Chlorpyrifos, Malathion, Acephate, Azamethiphos, Azinphos-ethyl, Azinphos-methyl, Cadusafos, Chlorethoxyfos, Chlorfenvinphos, Chlormephos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fonofos, Fosthiazate, Imicyafos, Isofenphos-methyl, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-ethyl, Profenofos, Propaphos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion Imidacloprid, Thiamethoxam, Acetamiprid, Clothianidin, Dinotefuran, Nitenpyram, Nithiozine, Nicotine, Thiacloprid, cyantraniliprole, carbamates, organophosphates, cyclodiene organochlorines, phenylpyrazoles (fiproles), pyrethroids, pyrethins, DDT Methoxychlor, Neonicotinoids, Nicotine, Sulfoximines, Butenolides, Mesoionics, Spinosyns, Avermectins, Milbernycins, Juvenile hormone analogues, Fenoxycarb, Pyriproxyfen, Alkyl halides, Chloropicrin, Fluorides, Borates, Tarter emetic, Methyl isothiocyanate generators, Pyridine azomethine derivatives, Pyropenes, Clofentezine, Diflovidazin, Hexythiazox, Etoxazole, Diafenthiuron, Organotin miticides, Propargite, Tetradifon, Pyrroles, Dinitrophenols, Sulfuramid, Nereistoxin analogues, Benzoylureas, Buprofezin, Cyromazine, Diacylhydrazines, Amitraz, Hydramethylnon, Acequinocyl, Fluacrypyrim, Bifenazate, METI acaricides and insecticides, Rotenone, Oxadiazines, Semicarbazones, Tetronic and Tetramic acid derivatives, Phosphides, Cyanides, Beta-ketonitrile derivatives, Carboxanilides, Diamides, Flonicamid, Meta-diamides Isoxazolines, Granuloviruses (GVs), Nucleopolyhedroviruses (NPVs), GS-omega/kappa HXTX-Hv1a peptide, Azadirachtin, Benzoximate, Bromopropylate, Chinomethionat, Dicofol, Lime sulfur, Mancozeb, Pyridalyl, Sulfur, Benzimidazoles, Dicarboximides, Pyridines, Pyrimidines, Triazoles, Acylalanines, Pyridine carboxamides, Anilino-pyrimidines, Quinone outside Inhibitors (QoI-fungicides), Phenylpyrroles, Quinolines, Hydroxyanilides, Toluamides, Cyanoacetamide-oximes, Dinitrophenyl crotonates, Phosphonates, Carboxylic Acid Amides (CAA-fungicides), M1 inorganic, M2 inorganic, M3 dithiocarbamates, M4 phthalimides, paraffinic oil, petroleum-based horticultural oils, palmitic oil, steric oil, linoleic oil, oleic oils, canola oil, soybean oil, oregano oil, tagetes oil, balsam fir oil, thyme oil, black pepper oil, mint oil, cedarwood oil, fish oil, jojoba oil, lavadin oil, castor oil, eucalyptus oil, ocimum oil, patchouli oil, citrus oil, artemisia oil, camphor oil, wintergreen oil, methyl eugenol oil, thymol oil, geranium oil, sesame oil, linseed oil, cottonseed oil, lemongrass oil, bergamot oil, mustard oil, orange oil, citronella oil, tea tree oil, neem oil, garlic oil, Bacillus sphaericus, Bacillus thuringiensis (e.g., Bacillus thuringiensis var. aizawai, Bacillus thuringiensis var. israelensis, Bacillus thuringiensis var. kurstaki, Bacillus thuringiensis var. sphaericus, Bacillus thuringiensis var. tenebrionensis) and the insecticidal proteins they produce (e.g., Cry1Ab, Cry1Ac, Cry1Fa, Cry1A.105, Cry2Ab, Vip3A, mCry3A, Cry3Ab, Cry3Bb, Cry34Ab1/Cr35Ab1, and as further exemplified in Crickmore, N., Baum, J., Bravo, A., Lereclus, D., Narva, K., Sampson, K., Schnepf, E., Sun, M. and Zeigler, D. R. “Bacillus thuringiensis toxin nomenclature” (2018)). Paenibacillus popilliae, Serratia entomophila, nuclear polyhedrosis viruses, granulosis viruses, non-occluded baculoviruses, Beauveria spp, Metarhizium, Entomophaga, Zoopthora, Paecilomyces fumosoroseus, Normuraea, Lecanicillium lecanii, Nosema, Thelohania, Vairimorpha, Steinernema spp, Heterorhabditis spp or any combination thereof, which may further comprise an active ingredient selected from the group consisting of azinphos-methyl, acephate, isoxathion, isofenphos, ethion, etrimfos, oxydemeton-methyl, oxydeprofos, quinalphos, chlorpyrifos, chlorpyrifos-methyl, chlorfenvin phos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafen thion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofen carb, carbaryl, carbosulfan, xylylcarb, thiodicarb, pirimicarb, fenobucarb, furathiocarb, propoxur, ben diocarb, benfuracarb, methomyl, metolcarb, XMC, carbofuran, aldoxycarb, oxamyl, acrin athrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cyper-methrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, ben sultap, acetamiprid, imidacloprid, clothianidin, dinotefuran, thiacloprid, thiamethoxam, nitenpyram, chlorfluazuron, difluben zuron, teflubenzuron, triflumuron, novaluron, noviflumuron, bistrifluoron, fluazuron, flucy-cloxuron, flufenoxuron, hexaflumuron, lufenuron, chromafen ozide, tebufenozide, halofen ozide, methoxyfen ozide, diofen olan, cyromazin e, pyriproxyfen, buprofezin, methop-rene, hydroprene, kinoprene, triazamate, endosulfan, chlorfenson, chlorobenzilate, dicofol, bromopropylate, acetoprole, flpronil, ethiprole, pyrethrin, rotenone, nicotinesulphate, spinosad, finpronil, spirotetramat abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, flu-azinam, flufenzin, hexythiazox, propargite, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, tri-adimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triti-conazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuari-mol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D, carbam, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tia-dinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine acetate, quintozene, tolylfluanid, anilazine, nitrothalisopropyl, fenitropan, dimethirimol, benthiazole, flumetover, mandipropamide, and penthiopyrad, or any combinations thereof.

In some embodiments, an RNAi molecule targeting vATPase-E, PTSA2, or SAR1 is supplied in the diet of a Coleopteran insect. For example, an RNAi molecule targeting vATPase-E, PTSA2, or SAR1 may be applied topically to a plant, or seeds (e.g. via soaking, coating, dusting or spraying), or cells of a plant may be engineered to express the RNAi molecule. RNAi molecules may also be supplied in another food or water source.

The plant may be any plant that is subject to infestation by a Coleopteran insect. In some embodiments, the plant is a Solanaceous plant (e.g., family Solanaceae). Examples of Solanaceous plants include, but are not limited to, potato plants (Solanum tuberosum), buffalo bur plants (Solanum rostratum), eggplant plants (Solanum melongena), tomato plants (Solanum lycopersicum), tobacco plants (Nicotiana tabacum), pepper plants (Capsicum annum) and woody nightshade plants (Solanum dulcamara).

Thus, in some embodiments, the methods comprise delivering to a plant (e.g., a potato plant) with an RNAi molecule targeting vATPase-E, PTSA2, or SAR1, for example, in an effective amount to suppress infestation of the plant by a Coleopteran insect (e.g., Colorado potato beetle). In other embodiments, the methods comprise delivering to a buffalo bur plant with an RNAi molecule targeting vATPase-E, PTSA2, or SAR1, for example, in an effective amount to suppress infestation of the plant by a Coleopteran insect (e.g., Colorado potato beetle). In yet other embodiments, the methods comprise delivering to an eggplant plant with an RNAi molecule targeting vATPase-E, PTSA2, or SAR1, for example, in an effective amount to suppress infestation of the plant by a Coleopteran insect (e.g., Colorado potato beetle). In still other embodiments, the methods comprise delivering to a tomato plant with an RNAi molecule targeting vATPase-E, PTSA2, or SAR1, for example, in an effective amount to suppress infestation of the plant by a Coleopteran insect (e.g., Colorado potato beetle). In further embodiments, the methods comprise delivering to a tobacco plant with an RNAi molecule targeting vATPase-E, PTSA2, or SAR1, for example, in an effective amount to suppress infestation of the plant by a Coleopteran insect (e.g., Colorado potato beetle). In additional embodiments, the methods comprise delivering to a pepper plant with an RNAi molecule targeting vATPase-E, PTSA2, or SAR1, for example, in an effective amount to suppress infestation of the plant by a Coleopteran insect (e.g., Colorado potato beetle).

Delivering to a plant (e.g., a part of a plant) and/or Coleopteran insect an RNAi molecule targeting vATPase-E, PTSA2, or SAR1 may include, for example, applying (e.g., soaking, coating, or dusting) the RNAi molecule or a composition comprising the RNAi molecule topically to any portion of a plant (e.g., roots, tubers, stem, branches, leaves, flower, etc.), ground (e.g., soil, dirt, grass, etc.), insect and/or diet of the insect. A delivering step may also include genetically engineering cells of a plant to express the RNAi molecule. A delivering step may also include exposing a plant or Coleopteran insect to an organism (e.g., virus, bacteria, fungus, etc.) that has been genetically engineered to express and/or deliver the RNAi molecule to the plant or Coleopteran insect.

An effective amount is the amount of an RNAi molecule targeting vATPase-E, PTSA2, or SAR1 required to confer a beneficial effect on infestation (e.g. death, cessation of feeding, inhibition of growth, development or reproduction) by a Coleopteran insect, either alone or in combination with one or more other additives. Beneficial effects include a reduction in infestation, for example, by at least 10%, at least 20%, at least 30%, at least 40%, at least 50%, at least 60%, at least 70%, at least 80%, or at least 90%, relative to a control. In some embodiments, the control is the absence of an insecticide and/or pesticide. In some embodiments, an effective amount of an RNAi molecule targeting vATPase-E, PTSA2, or SAR1 completely eliminates Coleopteran insect (e.g., Colorado potato beetle) infestation of a plant.

Effective amounts vary, as recognized by those skilled in the art, depending on the particular plant, the severity of the infestation, the duration of the infestation, previous exposure to insecticides and like factors within the knowledge and expertise of a practitioner. These factors are well known to those of ordinary skill in that art and can be addressed with no more than routine experimentation. It is generally preferred that lower effective concentrations be used, that is, the lowest concentration that provides control of an insect, to increase efficiency and decrease cost.

An effective amount of an RNAi molecule targeting vATPase-E, PTSA2, or SAR1 may also vary depending on the method of delivery.

In some embodiments, an effective amount of an RNAi molecule targeting vATPase-E, PTSA2, or SAR1 is expressed as micrograms (μg) of RNAi molecule targeting vATPase-E, PTSA2, or SAR1 per centimeter squared (cm²) of a surface of a plant or ground (e.g., soil, dirt, grass, etc.), i.e., μg/cm². Thus, in some embodiments, an effective amount of an RNAi molecule targeting vATPase-E, PTSA2, or SAR1 comprises 0.0001 μg/cm² to 10 μg/cm². In some embodiments, an effective amount of an RNAi molecule targeting vATPase-E, PTSA2, or SAR1 comprises 0.0001 μg/cm² to 9 μg/cm², 0.0001 μg/cm² to 8 μg/cm², 0.0001 μg/cm² to 7 μg/cm², 0.0001 μg/cm² to 6 μg/cm², 0.0001 μg/cm² to 5 μg/cm², 0.0001 μg/cm² to 4 μg/cm², 0.0001 μg/cm² to 3 μg/cm², 0.0001 μg/cm² to 2 μg/cm², 0.0001 μg/cm² to 1 μg/cm², 0.0001 μg/cm² to 0.1 μg/cm², or 0.0001 μg/cm² to 0.01 μg/cm². In some embodiments, and effective amount of an RNAi molecule targeting vATPase-E, PTSA2, or SAR1 comprises 0.001 μg/cm² to 10 μg/cm². In some embodiments, an effective amount of an RNAi molecule targeting SAR1 comprises 0.001 μg/cm² to 9 μg/cm², 0.001 μg/cm² to 8 μg/cm², 0.001 μg/cm² to 7 μg/cm², 0.001 μg/cm² to 6 μg/cm², 0.001 μg/cm² to 5 μg/cm², 0.001 μg/cm² to 4 μg/cm², 0.001 μg/cm² to 3 μg/cm², 0.001 μg/cm² to 2 μg/cm², 0.001 μg/cm² to 1 μg/cm², 0.001 μg/cm² to 0.1 μg/cm², or 0.001 μg/cm² to 0.01 μg/cm². In some embodiments, an effective amount of an RNAi molecule targeting vATPase-E, PTSA2, or SAR1 comprises 0.001 μg/cm² to 10 μg/cm², 0.01 μg/cm² to 10 μg/cm², 0.1 μg/cm² to 10 μg/cm², 1 μg/cm² to 10 μg/cm², 2 μg/cm² to 10 μg/cm², 3 μg/cm² to 10 μg/cm², 4 μg/cm² to 10 μg/cm², 5 μg/cm² to 10 μg/cm², 6 μg/cm² to 10 μg/cm², 7 μg/cm² to 10 μg/cm², 8 μg/cm² to 10 μg/cm², or 9 μg/cm² to 10 μg/cm². In some embodiments, an effective amount of an RNAi molecule targeting vATPase-E, PTSA2, or SAR1 comprises 0.01 μg/cm² to 10 μg/cm², 0.1 μg/cm² to 10 μg/cm², 1 μg/cm² to 10 μg/cm², 2 μg/cm² to 10 μg/cm², 3 μg/cm² to 10 μg/cm², 4 μg/cm² to 10 μg/cm², 5 μg/cm² to 10 μg/cm², 6 μg/cm² to 10 μg/cm², 7 μg/cm² to 10 μg/cm², 8 μg/cm² to 10 μg/cm², or 9 μg/cm² to 10 μg/cm².

In some embodiments, an effective amount of an RNAi molecule targeting vATPase-E, PTSA2, or SAR1 is expressed as grams (g) of RNAi molecule targeting vATPase-E, PTSA2, or SAR1 per acre (ac.) of a surface of a plant or ground (e.g., soil, dirt, grass, etc.), i.e., g/ac. Thus, in some embodiments, an effective amount of an RNAi molecule targeting vATPase-E, PTSA2, or SAR1 comprises 0.01 g/ac. to 100 g/ac. In some embodiments, an effective amount of an RNAi molecule targeting vATPase-E or PTSA2 comprises 0.1 g/ac. to 100 g/ac., 1 g/ac. to 100 g/ac., 10 g/ac. to 100 g/ac., 20 g/ac. to 100 g/ac., 30 g/ac. to 100 g/ac., 40 g/ac. to 100 g/ac., 50 g/ac. to 100 g/ac., 60 g/ac. to 100 g/ac., 70 g/ac. to 100 g/ac., 80 g/ac. to 100 g/ac., or 90 g/ac. to 100 g/ac. In some embodiments, and effective amount of an RNAi molecule targeting SAR1 comprises, 0.01 g/ac. to 90 g/ac., 0.01 g/ac. to 80 g/ac., 0.01 g/ac. to 70 g/ac., 0.01 g/ac. to 60 g/ac., 0.01 g/ac. to 50 g/ac., 0.01 g/ac. to 40 g/ac., 0.01 g/ac. to 30 g/ac., 0.01 g/ac. to 20 g/ac., 0.01 g/ac. to 10 g/ac., 0.01 g/ac. to 1 g/ac., or 0.01 g/ac. to 0.1 g/ac. In some embodiments, an effective amount of an RNAi molecule targeting vATPase-E, PTSA2, or SAR1 comprises 0.1 g/ac. to 100 g/ac., 1 g/ac. to 100 g/ac., 10 g/ac. to 100 g/ac., 20 g/ac. to 100 g/ac., 30 g/ac. to 100 g/ac., 40 g/ac. to 100 g/ac., 50 g/ac. to 100 g/ac., 60 g/ac. to 100 g/ac., 70 g/ac. to 100 g/ac., 80 g/ac. to 100 g/ac., or 90 g/ac. to 100 g/ac.

In some embodiments, the effectiveness of an RNAi molecule to control Coleopteran insects can be determined using the ability of the RNAi molecule to kill or cause death of an insect or population of insects. The rate of death in a population of insects may be determined by percent mortality (e.g., percent mortality over time). Generally, percent mortality of a population of insects reflects the percentage of insects in said population that have died as a result of the RNAi molecule (e.g., 75% mortality indicates that an RNAi molecule has killed 75% of the total insect population). In some embodiments, percent mortality is measured over time (e.g., over the course of a multi-day exposure of insects to an RNAi molecule). In some embodiments, percent mortality is measured after at least 3, 4, 5, 6, 7, 8, 9, 10, 15, or 20 days of exposure. In some embodiments, an RNAi molecule causes a percent mortality of at least 30%, 40%, 50%, 60%, 70%, 80%, 90%, 95%, 98%, or 100% of a Coleopteran insect population. In some embodiments, at least 30%, 40%, 50%, 60%, 70%, 80%, 90%, 95%, 98%, or 100% of a Coleopteran insect population are killed by an RNAi molecule that targets vATPase-E, PTSA2, or SAR1. In some embodiments, percent mortality of an RNAi molecule is compared to a control (e.g., a control molecule or untreated conditions). In some embodiments, percent mortality of an RNAi molecule is at least 30%, 40%, 50%, 60%, 70%, 80%, 90%, 100%, 120%, 150%, or 200% higher than a control (e.g., a control molecule or untreated conditions).

In some embodiments, the effectiveness of an RNAi molecule to control Coleopteran insects can be determined using the ability of the RNAi molecule to limit the leaf disc consumption of a Coleopteran insect or an insect population. Leaf disc consumption refers to the amount (e.g., percentage) of plant material (e.g., an eggplant leaf) that is consumed or eaten by an insect or population of insects. In some embodiments, an RNAi molecule causes at least a 10%, 20%, 30%, 40%, 50%, 60%, 70%, 80%, 90%, 95%, 98%, or 100% decrease in the leaf disc consumption by an insect or population of insects. In some embodiments, the ability of an RNAi molecule to decrease leaf disc consumption is compared relative to a control (e.g., a control molecule or untreated conditions). In some embodiments, leaf disc consumption is measured over time (e.g., over the course of a multi-day exposure of insects to an RNAi molecule). In some embodiments, leaf disc consumption is measured after 3, 4, 5, 6, 7, 8, 9, 10, or more days of exposure.

In some embodiments, the effectiveness of an RNAi molecule to control Coleopteran insects can be determined using the ability of the RNAi molecule to decrease percent plant consumption by a Coleopteran insect or an insect population. Percent plant consumption refers to the percentage of plant material (e.g., a potato leaf) that is destroyed (e.g., consumed) by an insect or population of insects. In some embodiments, an RNAi molecule causes at least a 10%, 20%, 30%, 40%, 50%, 60%, 70%, 80%, 90%, 95%, 98%, or 100% decrease in the percent plant defoliation by an insect or population of insects. In some embodiments, an RNAi molecule causes percent plant consumption to decrease below 40%, 30, 25%, 20%, 15%, 10%, 5%, 3%, or 1%. In some embodiments, percent plant consumption remains below 40%, 30, 25%, 20%, 15%, 10%, 5%, 3%, or 1% for at least 5, 6, 7, 8, 9, 10, 15, or 20 days following exposure of insects to an RNAi molecule. In some embodiments, the ability of an RNAi molecule to decrease percent plant consumption is compared relative to a control (e.g., a control molecule or untreated conditions). In some embodiments, percent plant consumption is measured over time (e.g., over the course of a multi-day exposure of insects to an RNAi molecule). In some embodiments, percent plant consumption is measured after 3, 4, 5, 6, 7, 8, 9, 10, or more days of exposure.

In some embodiments, an RNAi molecule targeting vATPase-E, PTSA2, or SAR1 may be formulated in a solution (e.g., that is applied to a surface of the Coleopteran insect and/or diet (e.g., food and/or water ingested), a plant or ground (e.g., soil, dirt, grass, etc.)). In some embodiments, the effective amount of the RNAi molecule targeting vATPase-E, PTSA2, or SAR1 in the solution is expressed as nanograms (ng) or micrograms (μg) of RNAi molecule targeting vATPase-E, PTSA2, or SAR1 per milliliter (ml) of the solution, i.e., ng/ml. Thus, in some embodiments, a solution comprises an RNAi molecule targeting vATPase-E, PTSA2, or SAR1 at a concentration of 10 ng/ml to 100 μg/ml. In some embodiments, a solution comprises an RNAi molecule targeting vATPase-E, PTSA2, or SAR1 at a concentration of 10 ng/ml to 100 μg/ml, 100 ng/ml to 100 μg/ml, 250 ng/ml to 100 μg/ml, 750 ng/ml to 100 μg/ml, 1000 ng/ml to 100 μg/ml, 10 μg/ml to 100 μg/ml, 25 μg/ml to 100 μg/ml, 50 μg/ml to 100 μg/ml, or 75 μg/ml to 100 μg/ml. In some embodiments, a solution comprises an RNAi molecule targeting vATPase-E, PTSA2, or SAR1 at a concentration of 10 ng/ml to 100 μg/ml, 10 ng/ml to 75 μg/ml, 10 ng/ml to 50 μg/ml, 10 ng/ml to 25 μg/ml, 10 ng/ml to 10 μg/ml, 10 ng/ml to 1000 ng/ml, 10 ng/ml to 1000 ng/ml, 10 ng/ml to 750 ng/ml, 10 ng/ml to 500 ng/ml, 10 ng/ml to 250 ng/ml, 10 ng/ml to 100 ng/ml, 10 ng/ml to 75 ng/ml, 10 ng/ml to 50 ng/ml, or 10 ng/ml to 25 ng/ml.

A solution, in some embodiments, comprises an RNAi molecule targeting vATPase-E, PTSA2, or SAR1 and at least one additional additive (e.g., a pesticide, surfactant or other non-pesticidal agent). In some embodiments, such a mixture comprises an RNAi molecule targeting vATPase-E or PTSA2 at a concentration of 0.0001 μg/ml to 10 μg/ml (e.g., that is applied to a surface of a plant and/or ground (e.g., soil, dirt, grass, etc.)). In some embodiments, such a mixture comprises an RNAi molecule targeting vATPase-E at a concentration of 0.0001 μg/ml to 9 μg/ml, 0.0001 μg/ml to 8 μg/ml, 0.0001 μg/ml to 7 μg/ml, 0.0001 μg/ml to 6 μg/ml, 0.0001 μg/ml to 5 μg/ml, 0.0001 μg/ml to 4 μg/ml, 0.0001 μg/ml to 3 μg/ml, 0.0001 μg/ml to 2 μg/ml, 0.0001 μg/ml to 1 μg/ml, 0.0001 μg/ml to 0.1 μg/ml, or 0.001 μg/ml to 0.01 μg/ml. In some embodiments, such a mixture comprises an RNAi molecule targeting PTSA2 or SAR1 at a concentration of 0.001 μg/ml to 10 μg/ml, 0.01 μg/ml to 10 μg/ml, 0.1 μg/ml to 10 μg/ml, 1 μg/ml to 10 μg/ml, 2 μg/ml to 10 μg/ml, 3 μg/ml to 10 μg/ml, 4 μg/ml to 10 μg/ml, 5 μg/ml to 10 μg/ml, 6 μg/ml to 10 μg/ml, 7 μg/ml to 10 μg/ml, 8 μg/ml to 10 μg/ml, or 9 μg/ml to 10 μg/ml. In some embodiments, such a mixture comprises an RNAi molecule targeting vATPase-E, PTSA2, or SAR1 at a concentration of 0.0001 μg/ml to 9 μg/ml, 0.0001 μg/ml to 8 μg/ml, 0.0001 μg/ml to 7 μg/ml, 0.0001 μg/ml to 6 μg/ml, 0.0001 μg/ml to 5 μg/ml, 0.0001 μg/ml to 4 μg/ml, 0.0001 μg/ml to 3 μg/ml, 0.0001 μg/ml to 2 μg/ml, 0.0001 μg/ml to 1 μg/ml, 0.0001 μg/ml to 0.1 μg/ml, 0.0001 μg/ml to 0.01 μg/ml, or 0.0001 μg/ml to 0.001 μg/ml.

In some embodiments, an RNAi molecule targeting vATPase-E, PTSA2, or SAR1 is provided in a diet of an insect. Thus, in some embodiments, an effective amount of an RNAi molecule targeting vATPase-E, PTSA2, or SAR1 is expressed as micrograms (μg) of RNAi molecule targeting vATPase-E, PTSA2, or SAR1 per milliliter (ml) of the diet of the insect, i.e., μg/ml. In some embodiments, the diet of an insect comprises an RNAi molecule targeting vATPase-E, PTSA2, or SAR1 at a concentration of 0.001 μg/ml to 10 μg/ml. In some embodiments, the diet of an insect comprises an RNAi molecule targeting vATPase-E, PTSA2, or SAR1 at a concentration of 0.0001 μg/ml to 9 μg/ml, 0.0001 μg/ml to 8 μg/ml, 0.0001 μg/ml to 7 μg/ml, 0.0001 μg/ml to 6 μg/ml, 0.0001 μg/ml to 5 μg/ml, 0.0001 μg/ml to 4 μg/ml, 0.0001 μg/ml to 3 μg/ml, 0.0001 μg/ml to 2 μg/ml, 0.0001 μg/ml to 1 μg/ml, 0.0001 μg/ml to 0.1 μg/ml, or 0.001 μg/ml to 0.01 μg/ml, 0.001 μg/ml to 9 μg/ml, 0.001 μg/ml to 8 μg/ml, 0.001 μg/ml to 7 μg/ml, 0.001 μg/ml to 6 μg/ml, 0.001 μg/ml to 5 μg/ml, 0.001 μg/ml to 4 μg/ml, 0.001 μg/ml to 3 μg/ml, 0.001 μg/ml to 2 μg/ml, 0.001 μg/ml to 1 μg/ml, 0.001 μg/ml to 0.1 μg/ml, or 0.001 μg/ml to 0.01 μg/ml. In some embodiments, the diet of an insect comprises an RNAi molecule targeting vATPase-E, PTSA2, or SAR1 at a concentration of 0.01 μg/ml to 10 μg/ml, 0.1 μg/ml to 10 μg/ml, 1 μg/ml to 10 μg/ml, 2 μg/ml to 10 μg/ml, 3 μg/ml to 10 μg/ml, 4 μg/ml to 10 μg/ml, 5 μg/ml to 10 μg/ml, 6 μg/ml to 10 μg/ml, 7 μg/ml to 10 μg/ml, 8 μg/ml to 10 μg/ml, or 9 μg/ml to 10 μg/ml.

The step of delivering to any portion of a plant (e.g., roots, tubers, stem, branches, leaves, flower, etc.), ground (e.g., soil, dirt, grass, etc.), insect and/or diet of the insect with an RNAi molecule targeting vATPase-E, PTSA2, or SAR1 may include a single application (single contact) or multiple applications (multiple contacts) of the RNAi molecule targeting vATPase-E, PTSA2, or SAR1 to the plant, ground (e.g., soil, dirt, grass, etc.), insect and/or diet of the insect. Delivery to a portion of a plant, insect and/or diet of the insect may be in the form of a spray (e.g., pressurized/aerosolized spray, pump) solid, (e.g. powder, pellet, bait), or liquid (e.g., homogeneous mixtures such as solutions and non-homogeneous mixtures such as suspensions (water and oil based), colloids, micelles, and emulsions). The period of time of contact may vary. In some embodiments, delivering comprises an exposure of an RNAi molecule targeting vATPase-E, PTSA2, or SAR1 with a portion of a plant and/or Coleopteran insect for a suitable period sufficient for reduction of growth, reproduction (e.g., fertility and/or fecundity), and/or feeding of the Coleopteran insect and/or death of the Coleopteran insect, if any.

In some embodiments, delivery of an RNAi molecule targeting vATPase-E, PTSA2, or SAR1 with a plant and/or Coleopteran insect is followed by ingestion and/or absorption of the RNAi molecule targeting vATPase-E, PTSA2, or SAR1 by the plant and/or Coleopteran insect. In some embodiments, ingestion of the RNAi molecule targeting vATPase-E, PTSA2, or SAR1 by the Coleopteran insect alters a biological function of the Coleopteran insect, thereby controlling infestation by the Coleopteran insect. Examples of altered biological function of the Coleopteran insect include, but are not limited to, reduced growth, reduced reproduction (e.g., fertility and/or fecundity), reduced feeding, decreased movement, decreased development, decreased cellular repair, and/or increased mortality.

In some embodiments, delivering comprises applying an RNAi molecule targeting vATPase-E, PTSA2, or SAR1 to a portion of the surface of a plant, a surface contacted by a Coleopteran insect (e.g., ground (e.g., soil, dirt, grass, etc.)), and/or the Coleopteran insect. In some embodiments, applying an RNAi molecule targeting vATPase-E, PTSA2, or SAR1 to a portion of a surface comprises spraying, coating, and/or dusting the surface or portion thereof. In some embodiments, applying an RNAi molecule targeting vATPase-E, PTSA2, or SAR1 RNA to a portion of a surface comprises ground drenching or applying the RNAi molecule as a granulated or powdered formulation to the soil adjacent to the roots of the plant.

In some embodiments delivering comprises contacting a seed with an RNAi molecule targeting vATPase-E, PTSA2, or SAR1. In some embodiments, contacting a seed with an RNAi molecule targeting vATPase-E, PTSA2, or SAR1 can be accomplished using any method known in the art which allows a suppressive amount of dsRNA to enter the seed. These examples include, but are not limited to, soaking, spraying or coating the seed with powder, emulsion, suspension, or solution. In some embodiments, a seed coating or a seed treatment composition comprises an RNAi molecule targeting vATPase-E, PTSA2, or SAR1 and at least one plant-enhancing agent, including but not limited to active substances intended to positively influence seed germination, plant emergence, plant growth, plant defense, plant development, and/or plant yield.

An RNAi molecule targeting vATPase-E, PTSA2, or SAR1 may be applied to any portion of a plant (e.g., roots, tubers, stem, branches, leaves, flower, etc.). In some embodiments, the RNAi molecule targeting vATPase-E, PTSA2, or SAR1 is contacted with an above-ground portion of a plant (e.g., a leaf) and/or with a below-ground portion of a plant (e.g., a root), which may include at least one in furrow formulation selected from the group consisting of a powder, granule, pellet, capsule, soluble liquid concentrate, spray(after dilution or concentrate), fog, in furrow, seed treatment, seed coating, insect diet, bait, drench, drip irrigation, or any other forms suited for applying to a furrow. Portions of a plant that may be contacted with the RNAi molecule targeting vATPase-E, PTSA2, or SAR1 described herein include, but are not limited to, leaf, stem, flower, fruit, shoot, root, seed, tuber, anther, stamen, or pollen. In some embodiments, RNAi is delivered mechanically, through high pressure spray or sandblasting.

In some embodiments, delivering comprises providing an RNAi molecule targeting vATPase-E, PTSA2, or SAR1 for dietary uptake by the Coleopteran insect. In some embodiments, contacting comprises providing an RNAi molecule targeting vATPase-E, PTSA2, or SAR1 that can be ingested or otherwise absorbed internally by the Coleopteran insect. In some embodiments, the RNAi molecule targeting vATPase-E, PTSA2, or SAR1 is provided in a diet for dietary uptake by the Coleopteran insect. In some embodiments, the RNAi molecule targeting vATPase-E, PTSA2, or SAR1 is provided in/on a plant or plant part, or topically applied to a plant or plant part (e.g., soaking, coating, dusting). In some embodiments, the RNAi molecule targeting vATPase-E, PTSA2, or SAR1 is expressed in a plant or plant part.

In some embodiments, delivering an RNAi molecule targeting vATPase-E, PTSA2, or SAR1 to a Coleopteran insect inhibits expression of (reduces or inhibits expression of) an endogenous complementary nucleotide sequence (e.g., RNA sequence) in the Coleopteran insect. In some embodiments, the endogenous complementary nucleotide sequence is an endogenous vATPase-E, PTSA2, or SAR1 sequence.

Consequences of inhibition can be confirmed by any appropriate assay to evaluate one or more properties of an insect, or by biochemical techniques that evaluate molecules indicative of vATPase-E, PTSA2, or SAR1 expression (e.g., RNA, protein). In some embodiments, the extent to which an RNAi molecule targeting vATPase-E, PTSA2, or SAR1 provided herein reduces levels of expression of vATPase-E, PTSA2, or SAR1 is evaluated by comparing expression levels (e.g., mRNA or protein levels of vATPase-E, PTSA2, or SAR1 to an appropriate control (e.g., a level of vATPase-E, PTSA2, or SAR1 expression in a cell or population of cells to which an RNAi molecule targeting vATPase-E, PTSA2, or SAR1 has not been delivered or to which a negative control has been delivered). In some embodiments, an appropriate control level of vATPase-E, PTSA2, or SAR1 expression may be a predetermined level or value, such that a control level need not be measured every time. The predetermined level or value can take a variety of forms. In some embodiments, a predetermined level or value can be single cut-off value, such as a median or mean.

In some embodiments, delivering an RNAi molecule targeting vATPase-E, PTSA2, or SAR1 as described herein results in a reduction in the level of vATPase-E, PTSA2, or SAR1 expression in a cell of an insect. In some embodiments, the reduction in levels of vATPase-E, PTSA2, or SAR1 expression may be a reduction by at least 10%, at least 20%, at least 30%, at least 40%, at least 50%, at least 60%, at least 70%, at least 80%, at least 90%, at least 95%, at least 98%, at least 99%, or 100% relative to a control level. In some embodiments, the control level is a level of vATPase-E, PTSA2, or SAR1 expression in a similar insect cell (or average level among a population of cells) not contacted with the RNAi molecule. In some embodiments, the control level is a level of vATPase-E, PTSA2, or SAR1 expression in a similar insect cell (or average level among a population of cells) contacted with an RNAi molecule targeting a gene not expressed by the insect cell, e.g., green fluorescent protein (GFP).

In some embodiments, the effect of delivering to a cell or insect an RNAi molecule targeting vATPase-E, PTSA2, or SAR1 is assessed after a finite period of time. For example, levels of vATPase-E, PTSA2, or SAR1 may be determined in a cell or insect at least 4 hours, 8 hours, 12 hours, 18 hours, 24 hours; or at least one, two, three, four, five, six, seven, or fourteen days after delivering to the cell or insect the RNAi molecule targeting vATPase-E, PTSA2, or SAR1.

In some embodiments, delivery of an RNAi molecule targeting vATPase-E, PTSA2, or SAR1 as described herein results in a reduction in the level of growth, reproduction (e.g., fertility and/or fecundity), and/or feeding of an insect. In some embodiments, the reduction in levels of growth, reproduction (e.g., fertility and/or fecundity), and/or feeding may be a reduction by at least 10%, at least 20%, at least 30%, at least 40%, at least 50%, at least 60%, at least 70%, at least 80%, at least 90%, at least 95%, at least 98%, at least 99%, or 100% relative to a control level. In some embodiments, the control level is a level of growth, reproduction (e.g., fertility and/or fecundity), and/or feeding of a similar insect not contacted with the RNAi molecule. In some embodiments, the control level is a level of growth, reproduction (e.g., fertility and/or fecundity), and/or feeding of a similar insect contacted with an RNAi molecule targeting a gene not expressed by the insect cell, e.g., GFP.

In some embodiments, delivery of an RNAi molecule targeting vATPase-E, PTSA2, or SAR1 as described herein results in an increase in mortality among a population of insects. In some embodiments, the increase in level of mortality may be an increase by at least 10%, at least 20%, at least 30%, at least 40%, at least 50%, at least 60%, at least 70%, at least 80%, at least 90%, at least 95%, at least 98%, at least 99%, or 100% relative to a control. In some embodiments, the control is mortality among a population of insects not contacted with the RNAi molecule. In some embodiments, the control is among a population of insects contacted with an RNAi molecule targeting a gene not expressed by the insect cell, e.g., GFP.

Aspects of the present disclosure provide plants that expresses an RNAi molecule targeting vATPase-E, PTSA2, or SAR1 as described herein. In some embodiments, DNA encoding an RNAi molecule targeting vATPase-E, PTSA2, or SAR1 provided herein is provided to a plant (seed or cells of a plant) such that the plant expresses the RNAi molecule targeting vATPase-E, PTSA2, or SAR1. In some embodiments, DNA encoding an RNAi molecule targeting vATPase-E, PTSA2, or SAR1 is expressed in a plant by transgenic expression, e.g., by stably integrating DNA encoding an RNAi molecule targeting vATPase-E, PTSA2, or SAR1 into a genome of a plant such that the plant expresses the RNAi molecule targeting vATPase-E, PTSA2, or SAR1.

### Methods of Producing RNAi Molecules

RNAi molecules targeting vATPase-E, PTSA2, or SAR1 as provided herein may be produced by any suitable method known in the art. Examples of methods for producing an RNAi molecule targeting vATPase-E, PTSA2, or SAR1 include, but are not limited to, in vitro transcription (IVT), chemical synthesis, expression in an organism (e.g., a plant), or expression in cell culture (e.g., a plant cell culture), and microbial fermentation.

RNAi molecules targeting vATPase-E, PTSA2, or SAR1 may be produced, in some embodiments, according to cell-free production methods described in International Application Publication WO 2017/176963 A1, published Oct. 12, 2017, entitled “Cell-Free Production of Ribonucleic Acid”; U.S. Provisional Application U.S. Ser. No. 62/571,071 filed Oct. 11, 2017, entitled “Methods and Compositions for Nucleoside Triphosphate and Ribonucleic Acid Production”; and International Application Publication WO 2019/075167 A1, published Apr. 18, 2019, entitled “Methods and Compositions for Nucleoside Triphosphate and Ribonucleic Acid Production”; each of which is incorporated herein by reference.

Any suitable DNA encoding RNAi molecules targeting vATPase-E, PTSA2, or SAR1 described herein may be used in the methods described herein. A DNA may be a single-stranded DNA (ssDNA) or a double-stranded DNA (dsDNA). In some embodiments, a DNA comprises one or more DNA expression cassette(s) that when transcribed produces a single-stranded RNA (ssRNA) molecule (e.g., that remains single-stranded or folds into an RNA hairpin) or complementary ssRNA molecules that anneal to produce the double-stranded RNA (dsRNA) molecule.

In some embodiments, a DNA comprises a promoter (e.g., an inducible promoter) operably linked to a nucleotide sequence encoding RNA that is complementary to a segment of vATPase-E, PTSA2, or SAR1, and optionally a terminator. In other embodiments, a DNA comprises a first promoter (e.g., an inducible promoter) operably linked to a nucleotide sequence encoding RNA that is complementary to a segment of vATPase-E, PTSA2, or SAR1, and optionally a terminator, and a second promoter (e.g., an inducible promoter) operably linked to a nucleotide sequence encoding a second RNA that is complementary to the first RNA, and optionally a terminator. In yet other embodiments, a DNA comprises a promoter (e.g., an inducible promoter) operably linked to a nucleotide sequence encoding a first region of an RNA, followed by one or more nucleotides of a loop region, followed by a second region of the RNA, and optionally followed by a terminator, wherein the first region of the RNA is complementary to a segment of vATPase-E, PTSA2, or SAR1 and the second region is complementary to the first region. In still other embodiments, a DNA comprises a first strand comprising a first promoter (e.g., an inducible promoter) operably linked to a nucleotide sequence encoding a first RNA that is complementary to a segment of vATPase-E, PTSA2, or SAR1, and optionally a terminator, and a second strand comprising a second promoter (e.g., an inducible promoter) operably linked to a nucleotide sequence encoding a second RNA that is complementary to the first RNA, and optionally a terminator wherein the first and second promoters are operably linked to the nucleotide sequence encoding a desired vATPase-E-targeting RNA, PTSA2-targeting RNA, or SAR1-targeting RNA and wherein the bidirectional transcription of the nucleotide sequence encoding the desired vATPase-E-targeting RNA, PTSA2-targeting RNA, or SAR1-targeting RNA results in complementary RNA molecules which anneal to form the dsRNA molecule.

A DNA is typically provided on a vector, such as a plasmid, although other template formats may be used (e.g., linear DNA generated by polymerase chain reaction (PCR), chemical synthesis, or other means known in the art). In some embodiments, more than one DNA is used in a reaction mixture. In some embodiments, 2, 3, 4, 5, or more different DNAs are used in a reaction mixture.

A promoter or terminator may be a naturally-occurring sequence or an engineered (e.g., synthetic) sequence. In some embodiments, an engineered sequence is modified to enhance transcriptional activity. In some embodiments, the promoter is a naturally-occurring sequence. In other embodiments, the promoter is an engineered sequence. In some embodiments, the terminator is a naturally-occurring sequence. In other embodiments, the terminator is an engineered sequence.

### EXAMPLES

In order that the invention described herein may be more fully understood, the following examples are set forth. The Examples described in this Application are offered to illustrate the methods, compositions, and systems provided herein and are not to be construed in any way as limiting their scope.

### Example 1: vATPase-E RNAi Composition Kills Colorado Potato Beetles

To evaluate the effect of vATPase-E RNAi polynucleotides (SEQ ID NOS: 6 or 7) on Colorado potato beetles (CPBs), compositions (e.g., comprising water) comprising either the vATPase-E1 or vATPase-E2 RNAi polynucleotide (hereafter, “GS 58 or GS 59”) is applied (at a concentration of 10 μg/cm²) onto the leaves of potato plants and allowed to dry. GS 58 comprises SEQ ID NO: 6; and GS 59 comprises SEQ ID NO: 7. Up to 100% of CPBs died following a 9 day exposure to the GS 58 or GS 59 covered potato plant leaves compared with no mortality following exposure to the control leaves. This increased mortality in response to exposure to GS 58 or GS 59 also resulted in a decrease of potato leaf consumption below 10% compared to CPBs exposed to the control. Percent potato leaf consumption refers to the percentage of potato leaf discs (punched out of potato leaves) following treatment of the discs with the RNAi composition and subsequent exposure of the discs to Colorado potato beetle, for example.

A dose-titration of the GS 58 composition was also performed to determine if a lower concentration of the GS 58 vATPase-E1 polynucleotide was equally effective in controlling CPBs. Up to 90%-100% of CPBs died following a 8 day exposure to GS 58 at 1.0 μg/cm², 0.1 μg/cm², and 0.01 μg/cm²; and about 50% of CPBs died following exposure to GS 58 at 0.001 μg/cm² compared to a control composition at 1.0 μg/cm². Potato leaf consumption also decreased to nearly 0% when CPBs were exposed to GS 58 at 1.0 μg/cm², 0.1 μg/cm², 0.01 μg/cm², and 0.001 μg/cm² compared to a control composition at 1.0 μg/cm².

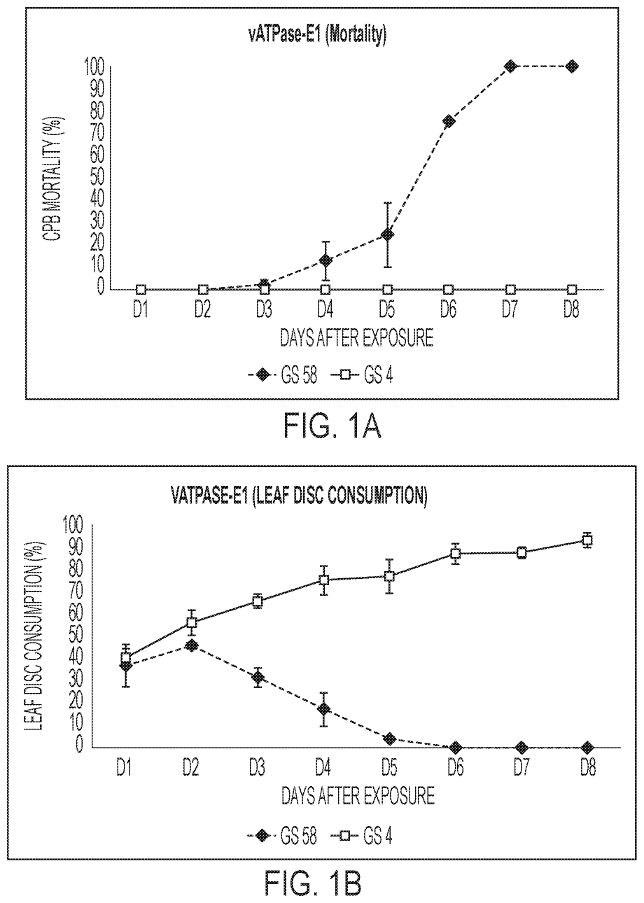

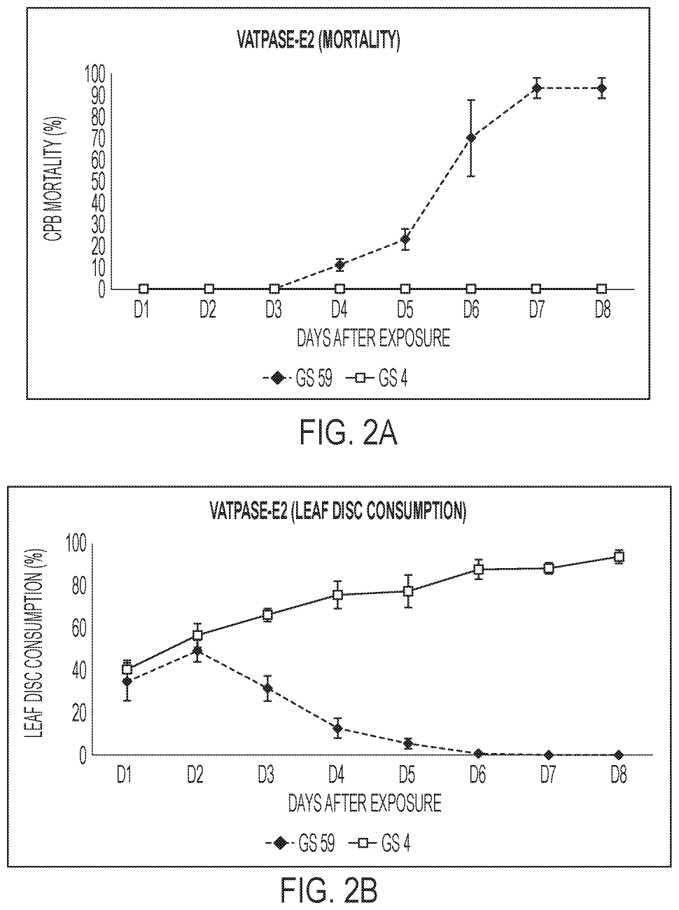

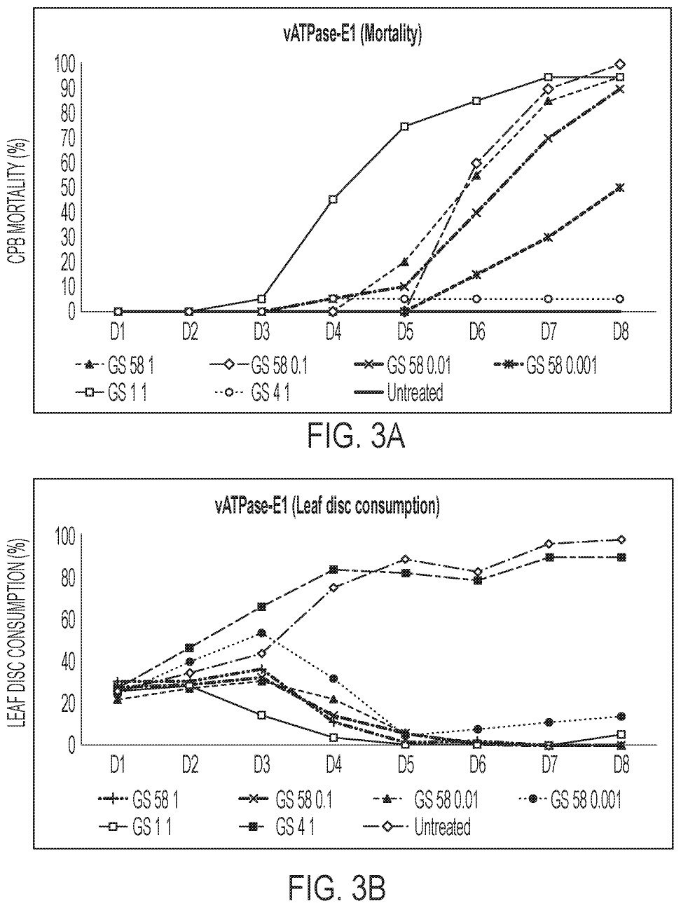

Exposure of CPBs to the vATPase-E1 polynucleotide GS 58 composition administered to potato leaves at a concentration of as low as 0.01 μg/cm² resulted in a 90% mortality and a 100% decreased potato leaf consumption compared to CPBs exposed to a control. See FIGS. 1-3.

### Example 2: PTSA2 RNAi Composition Kills Colorado Potato Beetles

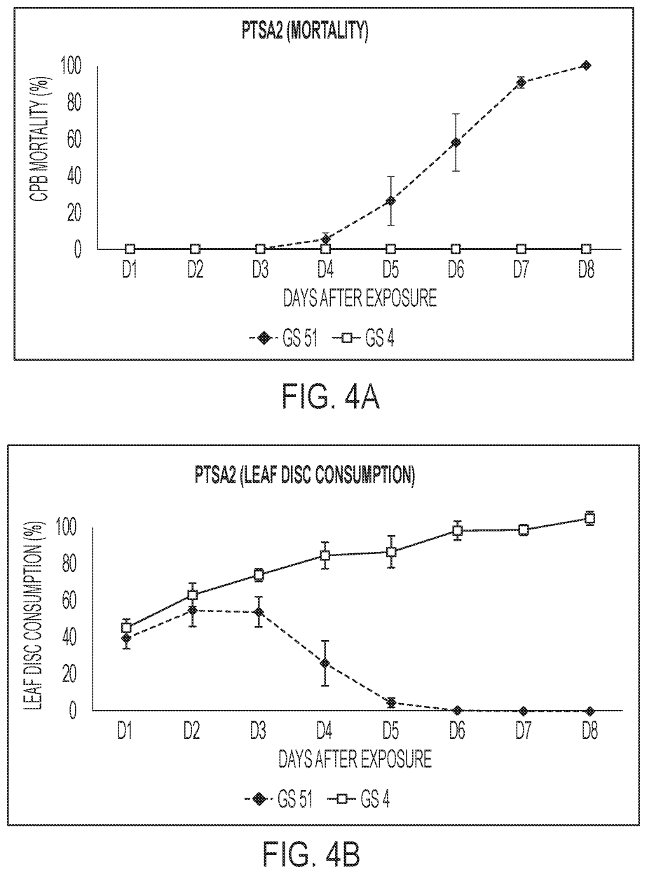

To evaluate the effect of PTSA2 RNAi polynucleotides (SEQ ID NO: 16) on Colorado potato beetles (CPBs), a composition (e.g., comprising water) comprising a PTSA2 RNAi polynucleotide (hereafter, “GS 51”) was applied (at a concentration of 10 μg/cm²) onto the leaves of potato plants and allowed to dry. Up to 100% of CPBs died following a 8 day exposure to the GS 51-covered potato plant leaves compared with no mortality of CPBs following exposure to the control leaves. This increased mortality in response to exposure to GS 51 also resulted in a decrease of potato leaf consumption below 5% at day 5 and stopped consumption at day 6 compared to CPBs exposed to the control. Percent potato leaf consumption refers to the percentage of potato leaf discs (punched out of potato leaves) following treatment of the discs with the RNAi composition and subsequent exposure of the discs to Colorado potato beetle, for example. See FIGS. 4A-4B.

### Example 3: SAR1 RNAi Composition Kills Colorado Potato Beetles

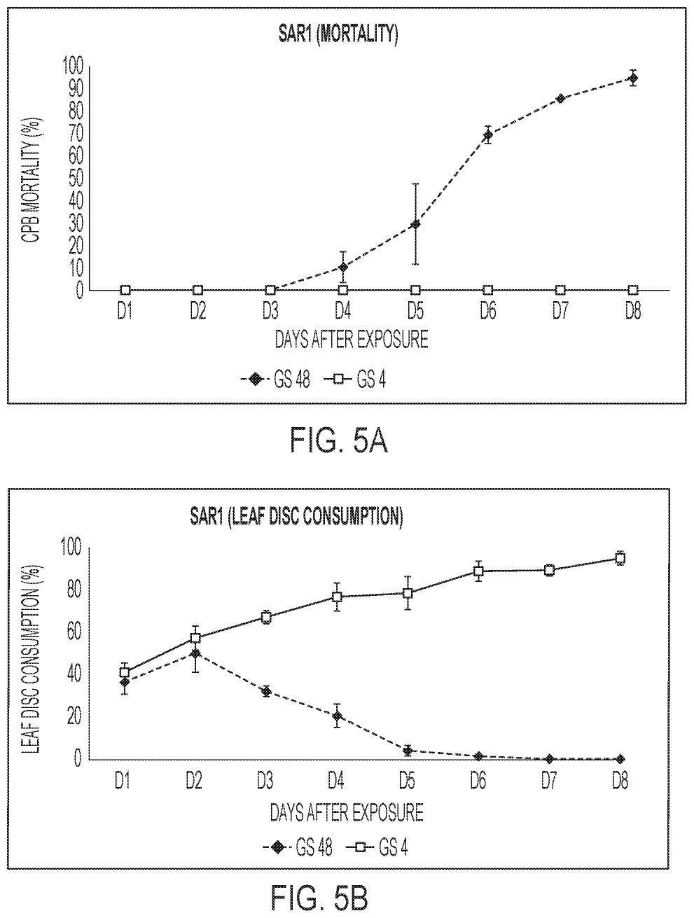

To evaluate the effect of SAR1 RNAi polynucleotides (SEQ ID NO: 23) on Colorado potato beetles (CPBs), a composition (e.g., comprising water) comprising a SAR1 RNAi polynucleotide (hereafter, “GS 48”) was applied (at a concentration of 10 μg/cm²) onto the leaves of potato plants and allowed to dry. Up to 90% of CPBs died following a 8-day exposure to the GL 47-covered potato plant leaves compared with less than 10% of CPBs that died following exposure to the control leaves. This increased mortality in response to exposure to GS 48 also resulted in a decrease of potato leaf consumption below 10% compared to CPBs exposed to the control. Percent potato leaf consumption refers to the percentage of potato leaf discs (punched out of potato leaves) following treatment of the discs with the RNAi composition and subsequent exposure of the discs to Colorado potato beetle, for example. See FIGS. 5A-5B

TABLE 1
vATPase Sequences, 5′ [symbol] 3

| | | | |
|---|---|---|---|
| | | | SEQ |
| | Length | | ID |
| Description | (bp) | Sequence | NO: |

DNA

| | | | |
|---|---|---|---|
| vATPase DNA | 1011 | CTGAAAAGTCACAAGACTTAACAGAAGTAAGTCATATGA | 1 |
| | bp | TCAATCAAATAAGCTTTGCGTTTCACGTCCGTAATTTGGG | |
| | | TGTCTCTTGTTAAGTGAAGGCGTTGTCTGTATCTTCATTA | |
| | | ACTTATATCTCTTGATAGTATTTCTTGAAAAAGTTCACCA | |
| | | TGGCTTTGAGCGACGTTGATGTACAAAAGCAGATCAAGC | |
| | | ATATGATGGCTTTCATTGAACAAGAGGCAAACGAAAAGG | |
| | | CAGAAGAAATCGATGCCAAGGCCGAGGAAGAATTTAATA | |
| | | TTGAAAAGGGGCGCCTTGTTCAGCAACAACGTCTCAAGA | |
| | | TTATGGAATATTATGAGAAGAAAGAGAAACAGGTCGAAC | |
| | | TCCAGAAAAAAATCCAATCGTCTAACATGTTGAATCAGG | |
| | | CTCGATTGAAAGTATTGAAGGTTAGGGAAGATCACGTTC | |
| | | GTACCGTACTAGAGGAGGCGCGTAAACGACTTGGTCAGG | |
| | | TCACAAACGACCAGGGAAAATATTCCCAAATCCTGGAAA | |
| | | GCCTCATTTTGCAGGGATTATATCAGCTTTTTGAGAAAGA | |
| | | TGTTACCATTCGAGTTCGGCCCCAGGACCGAGAACTGGTC | |
| | | AAATCCATCATTCCCACCGTCACGAACAAGTATAAAGAT | |
| | | GCCACCGGTAAGGACATCCATCTGAAAATTGATGACGAA | |
| | | ATCCATCTGTCCCAAGAAACCACCGGGGGAATCGACCTG | |
| | | CTGGCGCAGAAAAACAAAATCAAGATCAGCAATACTATG | |
| | | GAGGCTCGTCTGGAGCTGATTTCGCAGCAGTTGGTGCCAC | |
| | | AAATCCGTACGGCTCTGTTTGGACGTAACGTCAACAGGA | |
| | | AATTCACCGATTAGGCATTTATTTGGTGGTCAGTTTTGCA | |
| | | TGTTGTATTCTGCAATTCGCTGTAATTGAAACACTAAAGT | |
| | | GTTTCGGAAAGTTTTAGCTGAATTATGTTCATTAGGATAT | |
| | | CGTGGTTACTGGAACATTAATCTGTATATAACATATTCCT | |
| | | TGTTAATAGTATTTTTTTTGTTTTAT | |

RNA STRANDS

| | | | |
|---|---|---|---|
| vATPase mRNA | 1011 | CUGAAAAGUCACAAGACUUAACAGAAGUAAGUCAUAUG | 2 |
| | bases | AUCAAUCAAAUAAGCUUUGCGUUUCACGUCCGUAAUUU | |
| | | GGGUGUCUCUUGUUAAGUGAAGGCGUUGUCUGUAUCUU | |
| | | CAUUAACUUAUAUCUCUUGAUAGUAUUUCUUGAAAAAG | |
| | | UUCACCAUGGCUUUGAGCGACGUUGAUGUACAAAAGCA | |
| | | GAUCAAGCAUAUGAUGGCUUUCAUUGAACAAGAGGCAA | |
| | | ACGAAAAGGCAGAAGAAAUCGAUGCCAAGGCCGAGGAA | |
| | | GAAUUUAAUAUUGAAAAGGGGCGCCUUGUUCAGCAACA | |
| | | ACGUCUCAAGAUUAUGGAAUAUUAUGAGAAGAAAGAG | |
| | | AAACAGGUCGAACUCCAGAAAAAAAUCCAAUCGUCUAA | |
| | | CAUGUUGAAUCAGGCUCGAUUGAAAGUAUUGAAGGUUA | |
| | | GGGAAGAUCACGUUCGUACCGUACUAGAGGAGGCGCGU | |
| | | AAACGACUUGGUCAGGUCACAAACGACCAGGGAAAAUA | |
| | | UUCCCAAAUCCUGGAAAGCCUCAUUUUGCAGGGAUUAU | |
| | | AUCAGCUUUUUGAGAAAGAUGUUACCAUUCGAGUUCGG | |
| | | CCCCAGGACCGAGAACUGGUCAAAUCCAUCAUUCCCAC | |
| | | CGUCACGAACAAGUAUAAAGAUGCCACCGGUAAGGACA | |
| | | UCCAUCUGAAAAUUGAUGACGAAAUCCAUCUGUCCCAA | |
| | | GAAACCACCGGGGGAAUCGACCUGCUGGCGCAGAAAAA | |
| | | CAAAAUCAAGAUCAGCAAUACUAUGGAGGCUCGUCUGG | |
| | | AGCUGAUUUCGCAGCAGUUGGUGCCACAAAUCCGUACG | |
| | | GCUCUGUUUGGACGUAACGUCAACAGGAAAUUCACCGA | |
| | | UUAGGCAUUUAUUUGGUGGUCAGUUUUGCAUGUUGUA | |
| | | UUCUGCAAUUCGCUGUAAUUGAAACACUAAAGUGUUUC | |
| | | GGAAAGUUUUAGCUGAAUUAUGUUCAUUAGGAUAUCG | |
| | | UGGUUACUGGAACAUUAAUCUGUAUAUAACAUAUUCCU | |
| | | UGUUAAUAGUAUUUUUUUUGUUUUAU | |
| vATPase | 400 | UCAUAUGAUCAAUCAAAUAAGCUUUGCGUUUCACGUCC | 3 |
| GS 58 | bases | GUAAUUUGGGUGUCUCUUGUUAAGUGAAGGCGUUGUCU | |
| dsRNA | | GUAUCUUCAUUAACUUAUAUCUCUUGAUAGUAUUUCUU | |
| | | GAAAAAGUUCACCAUGGCUUUGAGCGACGUUGAUGUAC | |
| | | AAAAGCAGAUCAAGCAUAUGAUGGCUUUCAUUGAACAA | |
| | | GAGGCAAACGAAAAGGCAGAAGAAAUCGAUGCCAAGGC | |
| | | CGAGGAAGAAUUUAAUAUUGAAAAGGGGCGCCUUGUUC | |
| | | AGCAACAACGUCUCAAGAUUAUGGAAUAUUAUGAGAAG | |
| | | AAAGAGAAACAGGUCGAACUCCAGAAAAAAAUCCAAUC | |
| | | GUCUAACAUGUUGAAUCAGGCUCGAUUGAAAGUAUUGA | |
| | | AGGUUAGGGAAGAUCACGUU | |
| vATPase | 408 | CGUACCGUACUAGAGGAGGCGCGUAAACGACUUGGUCA | 4 |
| GS 59 | bases | GGUCACAAACGACCAGGGAAAAUAUUCCCAAAUCCUGG | |
| dsRNA | | AAAGCCUCAUUUUGCAGGGAUUAUAUCAGCUUUUUGAG | |
| | | AAAGAUGUUACCAUUCGAGUUCGGCCCCAGGACCGAGA | |
| | | ACUGGUCAAAUCCAUCAUUCCCACCGUCACGAACAAGU | |
| | | AUAAAGAUGCCACCGGUAAGGACAUCCAUCUGAAAAUU | |
| | | GAUGACGAAAUCCAUCUGUCCCAAGAAACCACCGGGGG | |
| | | AAUCGACCUGCUGGCGCAGAAAAACAAAAUCAAGAUCA | |
| | | GCAAUACUAUGGAGGCUCGUCUGGAGCUGAUUUCGCAG | |
| | | CAGUUGGUGCCACAAAUCCGUACGGCUCUGUUUGGACG | |
| | | UAACGUCAACAGGAAAUUCACCGAUUAG | |

REVERSE COMPLEMENT RNA STRANDS

| | | | |
|---|---|---|---|
| vATPase mRNA | 1011 | AUAAAACAAAAAAAAUACUAUUAACAAGGAAUAUGUU | 5 |
| reverse | bases | AUAUACAGAUUAAUGUUCCAGUAACCACGAUAUCCUAA | |
| complement | | UGAACAUAAUUCAGCUAAAACUUUCCGAAACACUUUAG | |
| | | UGUUUCAAUUACAGCGAAUUGCAGAAUACAACAUGCAA | |
| | | AACUGACCACCAAAUAAAUGCCUAAUCGGUGAAUUUCC | |
| | | UGUUGACGUUACGUCCAAACAGAGCCGUACGGAUUUGU | |
| | | GGCACCAACUGCUGCGAAAUCAGCUCCAGACGAGCCUC | |
| | | CAUAGUAUUGCUGAUCUUGAUUUUGUUUUUCUGCGCCA | |
| | | GCAGGUCGAUUCCCCCGGUGGUUUCUUGGGACAGAUGG | |
| | | AUUUCGUCAUCAAUUUUCAGAUGGAUGUCCUUACCGGU | |
| | | GGCAUCUUUAUACUUGUUCGUGACGGUGGGAAUGAUGG | |
| | | AUUUGACCAGUUCUCGGUCCUGGGGCCGAACUCGAAUG | |
| | | GUAACAUCUUUCUCAAAAAGCUGAUAUAAUCCCUGCAA | |
| | | AAUGAGGCUUUCCAGGAUUUGGGAAUAUUUUCCCUGGU | |
| | | CGUUUGUGACCUGACCAAGUCGUUUACGCGCCUCCUCU | |
| | | AGUACGGUACGAACGUGAUCUUCCCUAACCUUCAAUAC | |
| | | UUUCAAUCGAGCCUGAUUCAACAUGUUAGACGAUUGGA | |
| | | UUUUUUUCUGGAGUUCGACCUGUUUCUCUUUCUUCUCA | |
| | | UAAUAUUCCAUAAUCUUGAGACGUUGUUGCUGAACAAG | |
| | | GCGCCCCUUUUCAAUAUUAAAUUCUUCCUCGGCCUUGG | |
| | | CAUCGAUUUCUUCUGCCUUUUCGUUUGCCUCUUGUUCA | |
| | | AUGAAAGCCAUCAUAUGCUUGAUCUGCUUUUGUACAUC | |
| | | AACGUCGCUCAAAGCCAUGGUGAACUUUUUCAAGAAAU | |
| | | ACUAUCAAGAGAUAUAAGUUAAUGAAGAUACAGACAAC | |
| | | GCCUUCACUUAACAAGAGACACCCAAAUUACGGACGUG | |
| | | AAACGCAAAGCUUAUUUGAUUGAUCAUAUGACUUACUU | |
| | | CUGUUAAGUCUUGUGACUUUUCAG | |
| vATPase | 400 | AACGUGAUCUUCCCUAACCUUCAAUACUUUCAAUCGAG | 6 |
| GS 58 | bases | CCUGAUUCAACAUGUUAGACGAUUGGAUUUUUUUCUGG | |
| dsRNA | | AGUUCGACCUGUUUCUCUUUCUUCUCAUAAUAUUCCAU | |
| reverse | | AAUCUUGAGACGUUGUUGCUGAACAAGGCGCCCCUUUU | |
| complement | | CAAUAUUAAAUUCUUCCUCGGCCUUGGCAUCGAUUUCU | |
| | | UCUGCCUUUUCGUUUGCCUCUUGUUCAAUGAAAGCCAU | |
| | | CAUAUGCUUGAUCUGCUUUUGUACAUCAACGUCGCUCA | |
| | | AAGCCAUGGUGAACUUUUUCAAGAAAUACUAUCAAGAG | |
| | | AUAUAAGUUAAUGAAGAUACAGACAACGCCUUCACUUA | |
| | | ACAAGAGACACCCAAAUUACGGACGUGAAACGCAAAGC | |
| | | UUAUUUGAUUGAUCAUAUGA | |
| vATPase | 408 | CUAAUCGGUGAAUUUCCUGUUGACGUUACGUCCAAACA | 7 |
| GS 59 | bases | GAGCCGUACGGAUUUGUGGCACCAACUGCUGCGAAAUC | |
| dsRNA | | AGCUCCAGACGAGCCUCCAUAGUAUUGCUGAUCUUGAU | |
| reverse | | UUUGUUUUUCUGCGCCAGCAGGUCGAUUCCCCCGGUGG | |
| complement | | UUUCUUGGGACAGAUGGAUUUCGUCAUCAAUUUUCAGA | |
| | | UGGAUGUCCUUACCGGUGGCAUCUUUAUACUUGUUCGU | |
| | | GACGGUGGGAAUGAUGGAUUUGACCAGUUCUCGGUCCU | |
| | | GGGGCCGAACUCGAAUGGUAACAUCUUUCUCAAAAAGC | |
| | | UGAUAUAAUCCCUGCAAAAUGAGGCUUUCCAGGAUUUG | |
| | | GGAAUAUUUUCCCUGGUCGUUUGUGACCUGACCAAGUC | |
| | | GUUUACGCGCCUCCUCUAGUACGGUACG | |

TABLE 2
PTSA2 Sequences, 5′ [symbol] 3

| | | | |
|---|---|---|---|
| | | | SEQ |
| | Length | | ID |
| Description | (bp) | Sequence | NO: |

DNA

| | | | |
|---|---|---|---|
| PTSA2 DNA#1 | 856 | CAGACGTCAAAATCTGAATACCTATACCTCTGTGATTCTG | 8 |
| | bp | TGATTTTGTTTTTTAGTTGCAAGTAATCATGGCTTCGGAG | |
| | | CGGTATAGTTTTTCGCTGACAACTTTCAGTCCATCTGGAA | |
| | | AACTAGTTCAAATTGAATATGCCCTAGCTGCTGTAGCCGC | |
| | | TGGAGCTCCTTCAGTGGGCATTAAAGCTTCAAATGGTGTA | |
| | | GTTATCGCCACAGAAAACAAACATAAGTCGATCCTCTAT | |
| | | GAAGAACACAGTGTTCATAAAGTGGAAATGATTACAAAA | |
| | | CATATAGGAATGATATATTCTGGTATGGGACCTGATTATC | |
| | | GCTTGTTAGTGAAACAAGCTCGTAAAATGGCCCAACAGT | |
| | | ATTATCTAGTTTATCAAGAGCCTATACCAACAGTTCAACT | |
| | | CGTTCAACGAGTTGCCACTGTTATGCAAGAATATACTCAG | |
| | | TCCGGAGGAGTTAGGCCGTTTGGGGTTTCATTATTGATAT | |
| | | GTGGTTGGGACAGTGAACGACCATACTTATTTCAATGTGA | |
| | | TCCATCAGGAGCTTATTTTGCCTGGAAAGCTACTGCCATG | |
| | | GGCAAGAATTTCATCAATGGAAAAACATTTTTGGAAAAA | |
| | | AGATATAGCGAGGATTTGGAACTTGATGACGCAGTACAC | |
| | | ACAGCAATTCTGACGTTGAAGGAGAGTTTTGAAGGCCAA | |
| | | ATGACAGCGGACAACATTGAAGTGGGAATTTGTGATGAA | |
| | | GCAGGATTCAGGAGGCTAGATCCCTCTCATGTGAAGGAT | |
| | | TACCTAGCTAATATTCCATAAGGCATTTAGGTTATATAAC | |
| | | AAGATTTCTCTTAATTTTTTATGAAACTCATGTTTCACTTG | |
| | | AATAAAACCGGATTTGAAGGAAA | |
| PTSA2 DNA#2 | 1093 | ATATATTTATTTGGGAACTTTCGATGTTTAGAGACGATGT | 9 |
| | bp | AATGTCCATGTCAATGACCAATGCAAATAAAAGATACCA | |
| | | TGTAGGTATATGCTCTGTGATTCCATGATTTTTCCGATAC | |
| | | ATAATAAAGTTCAAATACTTCCGTTTATTTAAGGCAGTTA | |
| | | TTTTATTCACAAAAATTTTGTGAAATTTGAACAAGCCATG | |
| | | CTGAACCTTTTTCAATGCCTTAATGAGAAAAATCTTTTTG | |
| | | ACGTCTTGAGAAGAGCAAATAAAAGTCAGACGTCAAAAT | |
| | | CTGAATACCTATACCTCTGTGATTCTGTGATTTTGTTTTTT | |
| | | AGTTGCAAGTAATCATGGCTTCGGAGCGGTATAGTTTTTC | |
| | | GCTGACAACTTTCAGGTGATTATAATTCAATTATTGAAGG | |
| | | AACAGTATTCATATTGTTTTAATTTTCAGTCCATCTGGAA | |
| | | AACTAGTTCAAATTGAATATGCCCTAGCTGCTGTAGCCGC | |
| | | TGGAGCTCCTTCAGTGGGCATTAAAGGTATTTAACCTCTA | |
| | | CCTAGTGAATGATACATCATTTTCATGCACATAATTAAAT | |
| | | TTTTCTTATAGCTTCAAATGGTGTAGTTATCGCCACAGAA | |
| | | AACAAACATAAGTCGATCCTCTATGAAGAACACAGTGTT | |
| | | CATAAAGTGGAAATGATTACAAAACATATAGGAATGATA | |
| | | TATTCTGGTATGGGACCTGATTATCGCTTGTTAGTGAAAC | |
| | | AAGCTCGTAAAATGGCCCAACAGTATTATCTAGTTTATCA | |
| | | AGAGCCTATACCAACAGTTCAACTCGTTCAACGAGTTGCC | |
| | | ACTGTTATGCAAGAATATACTCAGTCCGGAGGAGTTAGG | |
| | | CCGTTTGGGGTTTCATTATTGATATGTGGTTGGGACAGTG | |
| | | AACGACCATACTTATTTCAATGTGATCCATCAGGAGCTTA | |
| | | TTTTGCCTGGAAAGCTACTGCCATGGGCAAGAATTTCATC | |
| | | AATGGAAAAACATTTTTGGAAAAAAGGTAACAGCAAATT | |
| | | TAAAAAACATGAAAATAGAAAATAATCAAACAAATCTCA | |
| | | TAATATTTATAGACGTAGACAAATTTTGAATGCTAATGAC | |
| | | GGGAAAGTAGTTTTTTTCT | |

RNA STRANDS

| | | | |
|---|---|---|---|
| PTSA2 mRNA#1 | 856 | CAGACGUCAAAAUCUGAAUACCUAUACCUCUGUGAUUC | 10 |
| | bases | UGUGAUUUUGUUUUUUAGUUGCAAGUAAUCAUGGCUUC | |
| | | GGAGCGGUAUAGUUUUUCGCUGACAACUUUCAGUCCAU | |
| | | CUGGAAAACUAGUUCAAAUUGAAUAUGCCCUAGCUGCU | |
| | | GUAGCCGCUGGAGCUCCUUCAGUGGGCAUUAAAGCUUC | |
| | | AAAUGGUGUAGUUAUCGCCACAGAAAACAAACAUAAGU | |
| | | CGAUCCUCUAUGAAGAACACAGUGUUCAUAAAGUGGAA | |
| | | AUGAUUACAAAACAUAUAGGAAUGAUAUAUUCUGGUA | |
| | | UGGGACCUGAUUAUCGCUUGUUAGUGAAACAAGCUCGU | |
| | | AAAAUGGCCCAACAGUAUUAUCUAGUUUAUCAAGAGCC | |
| | | UAUACCAACAGUUCAACUCGUUCAACGAGUUGCCACUG | |
| | | UUAUGCAAGAAUAUACUCAGUCCGGAGGAGUUAGGCCG | |
| | | UUUGGGGUUUCAUUAUUGAUAUGUGGUUGGGACAGUG | |
| | | AACGACCAUACUUAUUUCAAUGUGAUCCAUCAGGAGCU | |
| | | UAUUUUGCCUGGAAAGCUACUGCCAUGGGCAAGAAUUU | |
| | | CAUCAAUGGAAAAACAUUUUUGGAAAAAAGAUAUAGCG | |
| | | AGGAUUUGGAACUUGAUGACGCAGUACACACAGCAAUU | |
| | | CUGACGUUGAAGGAGAGUUUUGAAGGCCAAAUGACAGC | |
| | | GGACAACAUUGAAGUGGGAAUUUGUGAUGAAGCAGGA | |
| | | UUCAGGAGGCUAGAUCCCUCUCAUGUGAAGGAUUACCU | |
| | | AGCUAAUAUUCCAUAAGGCAUUUAGGUUAUAUAACAAG | |
| | | AUUUCUCUUAAUUUUUUAUGAAACUCAUGUUUCACUUG | |
| | | AAUAAAACCGGAUUUGAAGGAAA | |
| PTSA2 mRNA#2 | 1093 | AUAUAUUUAUUUGGGAACUUUCGAUGUUUAGAGACGA | 11 |
| | bases | UGUAAUGUCCAUGUCAAUGACCAAUGCAAAUAAAAGAU | |
| | | ACCAUGUAGGUAUAUGCUCUGUGAUUCCAUGAUUUUUC | |
| | | CGAUACAUAAUAAAGUUCAAAUACUUCCGUUUAUUUAA | |
| | | GGCAGUUAUUUUAUUCACAAAAAUUUUGUGAAAUUUG | |
| | | AACAAGCCAUGCUGAACCUUUUUCAAUGCCUUAAUGAG | |
| | | AAAAAUCUUUUUGACGUCUUGAGAAGAGCAAAUAAAAG | |
| | | UCAGACGUCAAAAUCUGAAUACCUAUACCUCUGUGAUU | |
| | | CUGUGAUUUUGUUUUUUAGUUGCAAGUAAUCAUGGCUU | |
| | | CGGAGCGGUAUAGUUUUUCGCUGACAACUUUCAGGUGA | |
| | | UUAUAAUUCAAUUAUUGAAGGAACAGUAUUCAUAUUG | |
| | | UUUUAAUUUUCAGUCCAUCUGGAAAACUAGUUCAAAUU | |
| | | GAAUAUGCCCUAGCUGCUGUAGCCGCUGGAGCUCCUUC | |
| | | AGUGGGCAUUAAAGGUAUUUAACCUCUACCUAGUGAAU | |
| | | GAUACAUCAUUUUCAUGCACAUAAUUAAAUUUUUCUUA | |
| | | UAGCUUCAAAUGGUGUAGUUAUCGCCACAGAAAACAAA | |
| | | CAUAAGUCGAUCCUCUAUGAAGAACACAGUGUUCAUAA | |
| | | AGUGGAAAUGAUUACAAAACAUAUAGGAAUGAUAUAU | |
| | | UCUGGUAUGGGACCUGAUUAUCGCUUGUUAGUGAAACA | |
| | | AGCUCGUAAAAUGGCCCAACAGUAUUAUCUAGUUUAUC | |
| | | AAGAGCCUAUACCAACAGUUCAACUCGUUCAACGAGUU | |
| | | GCCACUGUUAUGCAAGAAUAUACUCAGUCCGGAGGAGU | |
| | | UAGGCCGUUUGGGGUUUCAUUAUUGAUAUGUGGUUGG | |
| | | GACAGUGAACGACCAUACUUAUUUCAAUGUGAUCCAUC | |
| | | AGGAGCUUAUUUUGCCUGGAAAGCUACUGCCAUGGGCA | |
| | | AGAAUUUCAUCAAUGGAAAAACAUUUUUGGAAAAAAG | |
| | | GUAACAGCAAAUUUAAAAAACAUGAAAAUAGAAAAUA | |
| | | AUCAAACAAAUCUCAUAAUAUUUAUAGACGUAGACAAA | |
| | | UUUUGAAUGCUAAUGACGGGAAAGUAGUUUUUUUCU | |
| PTSA2 | 385 | UAAUCAUGGCUUCGGAGCGGUAUAGUUUUUCGCUGACA | 12 |
| dsRNA#1 | bases | ACUUUCAGGUGAUUAUAAUUCAAUUAUUGAAGGAACAG | |
| GS 51 | | UAUUCAUAUUGUUUUAAUUUUCAGUCCAUCUGGAAAAC | |
| | | UAGUUCAAAUUGAAUAUGCCCUAGCUGCUGUAGCCGCU | |
| | | GGAGCUCCUUCAGUGGGCAUUAAAGGUAUUUAACCUCU | |
| | | ACCUAGUGAAUGAUACAUCAUUUUCAUGCACAUAAUUA | |
| | | AAUUUUUCUUAUAGCUUCAAAUGGUGUAGUUAUCGCCA | |
| | | CAGAAAACAAACAUAAGUCGAUCCUCUAUGAAGAACAC | |
| | | AGUGUUCAUAAAGUGGAAAUGAUUACAAAACAUAUAG | |
| | | GAAUGAUAUAUUCUGGUAUGGGACCUGAUUAUCGCUUG | |
| | | UUAGUG | |
| PTSA2 | 388 | CAGACGUCAAAAUCUGAAUACCUAUACCUCUGUGAUUC | 13 |
| dsRNA#2 | bases | UGUGAUUUUGUUUUUUAGUUGCAAGUAAUCAUGGCUUC | |
| | | GGAGCGGUAUAGUUUUUCGCUGACAACUUUCAGUCCAU | |
| | | CUGGAAAACUAGUUCAAAUUGAAUAUGCCCUAGCUGCU | |
| | | GUAGCCGCUGGAGCUCCUUCAGUGGGCAUUAAAGCUUC | |
| | | AAAUGGUGUAGUUAUCGCCACAGAAAACAAACAUAAGU | |
| | | CGAUCCUCUAUGAAGAACACAGUGUUCAUAAAGUGGAA | |
| | | AUGAUUACAAAACAUAUAGGAAUGAUAUAUUCUGGUA | |
| | | UGGGACCUGAUUAUCGCUUGUUAGUGAAACAAGCUCGU | |
| | | AAAAUGGCCCAACAGUAUUAUCUAGUUUAUCAAGAGCC | |
| | | UAUACCAAC | |

REVERSE COMPLEMENT RNA STRANDS

| | | | |
|---|---|---|---|
| PTSA2 mRNA#1 | 856 | UUUCCUUCAAAUCCGGUUUUAUUCAAGUGAAACAUGAG | 14 |
| reverse | bases | UUUCAUAAAAAAUUAAGAGAAAUCUUGUUAUAUAACCU | |
| complement | | AAAUGCCUUAUGGAAUAUUAGCUAGGUAAUCCUUCACA | |
| | | UGAGAGGGAUCUAGCCUCCUGAAUCCUGCUUCAUCACA | |
| | | AAUUCCCACUUCAAUGUUGUCCGCUGUCAUUUGGCCUU | |
| | | CAAAACUCUCCUUCAACGUCAGAAUUGCUGUGUGUACU | |
| | | GCGUCAUCAAGUUCCAAAUCCUCGCUAUAUCUUUUUUC | |
| | | CAAAAAUGUUUUUCCAUUGAUGAAAUUCUUGCCCAUGG | |
| | | CAGUAGCUUUCCAGGCAAAAUAAGCUCCUGAUGGAUCA | |
| | | CAUUGAAAUAAGUAUGGUCGUUCACUGUCCCAACCACA | |
| | | UAUCAAUAAUGAAACCCCAAACGGCCUAACUCCUCCGG | |
| | | ACUGAGUAUAUUCUUGCAUAACAGUGGCAACUCGUUGA | |
| | | ACGAGUUGAACUGUUGGUAUAGGCUCUUGAUAAACUAG | |
| | | AUAAUACUGUUGGGCCAUUUUACGAGCUUGUUUCACUA | |
| | | ACAAGCGAUAAUCAGGUCCCAUACCAGAAUAUAUCAUU | |
| | | CCUAUAUGUUUUGUAAUCAUUUCCACUUUAUGAACACU | |
| | | GUGUUCUUCAUAGAGGAUCGACUUAUGUUUGUUUUCUG | |
| | | UGGCGAUAACUACACCAUUUGAAGCUUUAAUGCCCACU | |
| | | GAAGGAGCUCCAGCGGCUACAGCAGCUAGGGCAUAUUC | |
| | | AAUUUGAACUAGUUUUCCAGAUGGACUGAAAGUUGUCA | |
| | | GCGAAAAACUAUACCGCUCCGAAGCCAUGAUUACUUGC | |
| | | AACUAAAAAACAAAAUCACAGAAUCACAGAGGUAUAGG | |
| | | UAUUCAGAUUUUGACGUCUG | |
| PTSA2 mRNA#2 | 1093 | AGAAAAAAACUACUUUCCCGUCAUUAGCAUUCAAAAUU | 15 |
| reverse | bases | UGUCUACGUCUAUAAAUAUUAUGAGAUUUGUUUGAUU | |
| complement | | AUUUUCUAUUUUCAUGUUUUUUAAAUUUGCUGUUACCU | |
| | | UUUUUCCAAAAAUGUUUUUCCAUUGAUGAAAUUCUUGC | |
| | | CCAUGGCAGUAGCUUUCCAGGCAAAAUAAGCUCCUGAU | |
| | | GGAUCACAUUGAAAUAAGUAUGGUCGUUCACUGUCCCA | |
| | | ACCACAUAUCAAUAAUGAAACCCCAAACGGCCUAACUC | |
| | | CUCCGGACUGAGUAUAUUCUUGCAUAACAGUGGCAACU | |
| | | CGUUGAACGAGUUGAACUGUUGGUAUAGGCUCUUGAUA | |
| | | AACUAGAUAAUACUGUUGGGCCAUUUUACGAGCUUGUU | |
| | | UCACUAACAAGCGAUAAUCAGGUCCCAUACCAGAAUAU | |
| | | AUCAUUCCUAUAUGUUUUGUAAUCAUUUCCACUUUAUG | |
| | | AACACUGUGUUCUUCAUAGAGGAUCGACUUAUGUUUGU | |
| | | UUUCUGUGGCGAUAACUACACCAUUUGAAGCUAUAAGA | |
| | | AAAAUUUAAUUAUGUGCAUGAAAAUGAUGUAUCAUUC | |
| | | ACUAGGUAGAGGUUAAAUACCUUUAAUGCCCACUGAAG | |
| | | GAGCUCCAGCGGCUACAGCAGCUAGGGCAUAUUCAAUU | |
| | | UGAACUAGUUUUCCAGAUGGACUGAAAAUUAAAACAAU | |
| | | AUGAAUACUGUUCCUUCAAUAAUUGAAUUAUAAUCACC | |
| | | UGAAAGUUGUCAGCGAAAAACUAUACCGCUCCGAAGCC | |
| | | AUGAUUACUUGCAACUAAAAAACAAAAUCACAGAAUCA | |
| | | CAGAGGUAUAGGUAUUCAGAUUUUGACGUCUGACUUUU | |
| | | AUUUGCUCUUCUCAAGACGUCAAAAAGAUUUUUCUCAU | |
| | | UAAGGCAUUGAAAAAGGUUCAGCAUGGCUUGUUCAAAU | |
| | | UUCACAAAAUUUUUGUGAAUAAAAUAACUGCCUUAAAU | |
| | | AAACGGAAGUAUUUGAACUUUAUUAUGUAUCGGAAAA | |
| | | AUCAUGGAAUCACAGAGCAUAUACCUACAUGGUAUCUU | |
| | | UUAUUUGCAUUGGUCAUUGACAUGGACAUUACAUCGUC | |
| | | UCUAAACAUCGAAAGUUCCCAAAUAAAUAUAU | |
| PTSA2 | 385 | CACUAACAAGCGAUAAUCAGGUCCCAUACCAGAAUAUA | 16 |
| dsRNA#1 | bases | UCAUUCCUAUAUGUUUUGUAAUCAUUUCCACUUUAUGA | |
| reverse | | ACACUGUGUUCUUCAUAGAGGAUCGACUUAUGUUUGUU | |
| complement | | UUCUGUGGCGAUAACUACACCAUUUGAAGCUAUAAGAA | |
| GS 51 | | AAAUUUAAUUAUGUGCAUGAAAAUGAUGUAUCAUUCAC | |
| | | UAGGUAGAGGUUAAAUACCUUUAAUGCCCACUGAAGGA | |
| | | GCUCCAGCGGCUACAGCAGCUAGGGCAUAUUCAAUUUG | |
| | | AACUAGUUUUCCAGAUGGACUGAAAAUUAAAACAAUAU | |
| | | GAAUACUGUUCCUUCAAUAAUUGAAUUAUAAUCACCUG | |
| | | AAAGUUGUCAGCGAAAAACUAUACCGCUCCGAAGCCAU | |
| | | GAUUA | |
| PTSA2 | 388 | GUUGGUAUAGGCUCUUGAUAAACUAGAUAAUACUGUUG | 17 |
| dsRNA#2 | bases | GGCCAUUUUACGAGCUUGUUUCACUAACAAGCGAUAAU | |
| reverse | | CAGGUCCCAUACCAGAAUAUAUCAUUCCUAUAUGUUUU | |
| complement | | GUAAUCAUUUCCACUUUAUGAACACUGUGUUCUUCAUA | |
| | | GAGGAUCGACUUAUGUUUGUUUUCUGUGGCGAUAACUA | |
| | | CACCAUUUGAAGCUUUAAUGCCCACUGAAGGAGCUCCA | |
| | | GCGGCUACAGCAGCUAGGGCAUAUUCAAUUUGAACUAG | |
| | | UUUUCCAGAUGGACUGAAAGUUGUCAGCGAAAAACUAU | |
| | | ACCGCUCCGAAGCCAUGAUUACUUGCAACUAAAAAACA | |
| | | AAAUCACAGAAUCACAGAGGUAUAGGUAUUCAGAUUUU | |
| | | GACGUCUG | |

TABLE 3
SAR1 Sequences, 5′ [symbol] 3

| | | | |
|---|---|---|---|
| | | | SEQ |
| | Length | | ID |
| Description | (bp) | Sequence | NO: |

DNA

| | | | |
|---|---|---|---|
| SAR1 DNA#1 | 1010 | CGTCATTCTGCCAACTTCTCCTTTACTGTCAACCATAAGT | 18 |
| | bp | AACAGAACTGTTACTATCAAATTAAAAAACTCTTAAAAT | |
| | | AATAATTGGGCGATTTTAAAGACTTTTCTGTAGTGAATAT | |
| | | CAACTTTACATATTTTTTGTGGGATAATTGGTCGTTTCCA | |
| | | ACGTTAGTTCAAATTACAAAAATGTTCATCTGGGATTGGT | |
| | | TTACCGGTGTACTTGGTTACTTGGGATTATGGAAGAAATC | |
| | | AGGCAAGCTGCTTTTCCTAGGCCTGGACAATGCTGGAAA | |
| | | AACAACTTTACTGCATATGCTCAAAGATGATAGGCTGGC | |
| | | ACAGCATCTTCCCACTTTACATCCTAGTAAGGCTCTTAAC | |
| | | ATTTTTCATAATGAAATGTCCAGTCCACATCACATGCTTC | |
| | | ACCTTCATTTTTGTAGAAGATCATACCCCCGAAAAACATT | |
| | | AAGGAACTTCGATTCCTCCACAAAAAATGTCTCCAAATAT | |
| | | GCTAACCTTATTTTTCTCTCTACAGCAAGGCGAGTGTGGA | |
| | | AGGACTACTTCCCTGCTGTTGACGCCATTGTCTTTTTAGT | |
| | | AGATGCCAATGATAGGAACAGATTCAAAGAGAGTAAACA | |
| | | AGAATTGGATTCGCTACTCACAGACGAGACTCTTTCGAAT | |
| | | TGTCCGGTTCTTATATTAGGTAACAAAATTGATTTGCCTC | |
| | | AAGCAGCTTCGGAAGACGAACTAAGAAATTACTATGCCT | |
| | | TGTATGGCCAAACAACTGGAAAGGGAAAGGTGGCAAGGT | |
| | | CCGATTTGCCAGGACGTCCCCTCGAGCTCTTCATGTGCTC | |
| | | CATTCTGAAGAGGCAGGGATACGGAGAAGGTTTCCGTTG | |
| | | GCTGGCCCAGTACATCGACTGAAGTTTTTTTGTGCGAGTG | |
| | | GATTCGTTAAGGTGTACCTACACGGTATTCTTTCATGAAC | |
| | | AAGGTTTTTCTATATCAATTCATATGTGACCTTTTTTTTTC | |
| | | GGTATAATTGGTTTTTTATCGTGTAGTATATATAAAAAAA | |
| | | ATAGTTTTAATAGTAG | |

RNA STRANDS

| | | | |
|---|---|---|---|
| SAR1 mRNA | 1010 | CGUCAUUCUGCCAACUUCUCCUUUACUGUCAACCAUAA | 19 |
| | bases | GUAACAGAACUGUUACUAUCAAAUUAAAAAACUCUUAA | |
| | | AAUAAUAAUUGGGCGAUUUUAAAGACUUUUCUGUAGU | |
| | | GAAUAUCAACUUUACAUAUUUUUUGUGGGAUAAUUGG | |
| | | UCGUUUCCAACGUUAGUUCAAAUUACAAAAAUGUUCAU | |
| | | CUGGGAUUGGUUUACCGGUGUACUUGGUUACUUGGGAU | |
| | | UAUGGAAGAAAUCAGGCAAGCUGCUUUUCCUAGGCCUG | |
| | | GACAAUGCUGGAAAAACAACUUUACUGCAUAUGCUCAA | |
| | | AGAUGAUAGGCUGGCACAGCAUCUUCCCACUUUACAUC | |
| | | CUAGUAAGGCUCUUAACAUUUUUCAUAAUGAAAUGUCC | |
| | | AGUCCACAUCACAUGCUUCACCUUCAUUUUUGUAGAAG | |
| | | AUCAUACCCCCGAAAAACAUUAAGGAACUUCGAUUCCU | |
| | | CCACAAAAAAUGUCUCCAAAUAUGCUAACCUUAUUUUU | |
| | | CUCUCUACAGCAAGGCGAGUGUGGAAGGACUACUUCCC | |
| | | UGCUGUUGACGCCAUUGUCUUUUUAGUAGAUGCCAAUG | |
| | | AUAGGAACAGAUUCAAAGAGAGUAAACAAGAAUUGGA | |
| | | UUCGCUACUCACAGACGAGACUCUUUCGAAUUGUCCGG | |
| | | UUCUUAUAUUAGGUAACAAAAUUGAUUUGCCUCAAGCA | |
| | | GCUUCGGAAGACGAACUAAGAAAUUACUAUGCCUUGUA | |
| | | UGGCCAAACAACUGGAAAGGGAAAGGUGGCAAGGUCCG | |
| | | AUUUGCCAGGACGUCCCCUCGAGCUCUUCAUGUGCUCC | |
| | | AUUCUGAAGAGGCAGGGAUACGGAGAAGGUUUCCGUUG | |
| | | GCUGGCCCAGUACAUCGACUGAAGUUUUUUUGUGCGAG | |
| | | UGGAUUCGUUAAGGUGUACCUACACGGUAUUCUUUCAU | |
| | | GAACAAGGUUUUUCUAUAUCAAUUCAUAUGUGACCUUU | |
| | | UUUUUUCGGUAUAAUUGGUUUUUUAUCGUGUAGUAUA | |
| | | UAUAAAAAAAAUAGUUUUAAUAGUAG | |
| SAR1 | 600 | GCAAUUGUUGAUGAAACGCCGGACAUACAAUUUACAGC | 20 |
| dsRNA#1 | bases | CACAGCGAGGCUAUUUAUUCCGGAAAAUAAUUUUGACA | |
| GS 48 | | CAUGGACAGGUAUAUUUCUUUCAACAAACAUCAAUAGA | |
| | | AUGAAAUGUCCAGUCCACAUCACAUGCUUCACCUUCAU | |
| | | UUUUGUAGAAGAUCAUACCCCCGAAAAACAUUAAGGAA | |
| | | CUUCGAUUCCUCCACAAAAAAUGUCUCCAAAUAUGCUA | |
| | | ACCUUAUUUUUCUCUCUACAGCAAGGCGAGUGUGGAAG | |
| | | GACUACUUCCCUGCUGUUGACGCCAUUGUCUUUUUAGU | |
| | | AGAUGCCAAUGAUAGGAACAGAUUCAAAGAGAGUAAAC | |
| | | AAGAAUUGGAUUCGCUACUCACAGACGAGACUCUUUCG | |
| | | AAUUGUCCGGUUCUUAUAUUAGGUAACAAAAUUGAUUU | |
| | | GCCUCAAGCAGCUUCGGAAGACGAACUAAGAAAUUACU | |
| | | AUGCCUUGUAUGGCCAAACAACUGGAAAGGUGAGUGUA | |
| | | AUUUUAUUAUUAAUGUUGAGUAAGGAGUUUUGCAUGA | |
| | | GAAAUAUUUUAUUUCGAUCAUUAGGUGGUCAUUUUGG | |
| | | AACGAUUCUUGGUGUUAAAAGUGUAUAGGAGG | |
| SAR1 | 373 | AAUGAAAUGUCCAGUCCACAUCACAUGCUUCACCUUCA | 21 |
| dsRNA#2 | bases | UUUUUGUAGAAGAUCAUACCCCCGAAAAACAUUAAGGA | |
| | | ACUUCGAUUCCUCCACAAAAAAUGUCUCCAAAUAUGCU | |
| | | AACCUUAUUUUUCUCUCUACAGCAAGGCGAGUGUGGAA | |
| | | GGACUACUUCCCUGCUGUUGACGCCAUUGUCUUUUUAG | |
| | | UAGAUGCCAAUGAUAGGAACAGAUUCAAAGAGAGUAAA | |
| | | CAAGAAUUGGAUUCGCUACUCACAGACGAGACUCUUUC | |
| | | GAAUUGUCCGGUUCUUAUAUUAGGUAACAAAAUUGAUU | |
| | | UGCCUCAAGCAGCUUCGGAAGACGAACUAAGAAAUUAC | |
| | | UAUGCCUUGUAUGGCCAAACAACUGGAAAGG | |

REVERSE COMPLEMENT RNA STRANDS

| | | | |
|---|---|---|---|
| SAR1 mRNA | 1010 | CUACUAUUAAAACUAUUUUUUUUAUAUAUACUACACGA | 22 |
| reverse | bases | UAAAAAACCAAUUAUACCGAAAAAAAAAGGUCACAUAU | |
| complement | | GAAUUGAUAUAGAAAAACCUUGUUCAUGAAAGAAUACC | |
| | | GUGUAGGUACACCUUAACGAAUCCACUCGCACAAAAAA | |
| | | ACUUCAGUCGAUGUACUGGGCCAGCCAACGGAAACCUU | |
| | | CUCCGUAUCCCUGCCUCUUCAGAAUGGAGCACAUGAAG | |
| | | AGCUCGAGGGGACGUCCUGGCAAAUCGGACCUUGCCAC | |
| | | CUUUCCCUUUCCAGUUGUUUGGCCAUACAAGGCAUAGU | |
| | | AAUUUCUUAGUUCGUCUUCCGAAGCUGCUUGAGGCAAA | |
| | | UCAAUUUUGUUACCUAAUAUAAGAACCGGACAAUUCGA | |
| | | AAGAGUCUCGUCUGUGAGUAGCGAAUCCAAUUCUUGUU | |
| | | UACUCUCUUUGAAUCUGUUCCUAUCAUUGGCAUCUACU | |
| | | AAAAAGACAAUGGCGUCAACAGCAGGGAAGUAGUCCUU | |
| | | CCACACUCGCCUUGCUGUAGAGAGAAAAAUAAGGUUAG | |
| | | CAUAUUUGGAGACAUUUUUUGUGGAGGAAUCGAAGUUC | |
| | | CUUAAUGUUUUUCGGGGGUAUGAUCUUCUACAAAAAUG | |
| | | AAGGUGAAGCAUGUGAUGUGGACUGGACAUUUCAUUAU | |
| | | GAAAAAUGUUAAGAGCCUUACUAGGAUGUAAAGUGGG | |
| | | AAGAUGCUGUGCCAGCCUAUCAUCUUUGAGCAUAUGCA | |
| | | GUAAAGUUGUUUUUCCAGCAUUGUCCAGGCCUAGGAAA | |
| | | AGCAGCUUGCCUGAUUUCUUCCAUAAUCCCAAGUAACC | |
| | | AAGUACACCGGUAAACCAAUCCCAGAUGAACAUUUUUG | |
| | | UAAUUUGAACUAACGUUGGAAACGACCAAUUAUCCCAC | |
| | | AAAAAAUAUGUAAAGUUGAUAUUCACUACAGAAAAGUC | |
| | | UUUAAAAUCGCCCAAUUAUUAUUUUAAGAGUUUUUUAA | |
| | | UUUGAUAGUAACAGUUCUGUUACUUAUGGUUGACAGUA | |
| | | AAGGAGAAGUUGGCAGAAUGACG | |
| SAR1 | 600 | CCUCCUAUACACUUUUAACACCAAGAAUCGUUCCAAAA | 23 |
| dsRNA#1 | bases | UGACCACCUAAUGAUCGAAAUAAAAUAUUUCUCAUGCA | |
| reverse | | AAACUCCUUACUCAACAUUAAUAAUAAAAUUACACUCA | |
| complement | | CCUUUCCAGUUGUUUGGCCAUACAAGGCAUAGUAAUUU | |
| GS 48 | | CUUAGUUCGUCUUCCGAAGCUGCUUGAGGCAAAUCAAU | |
| | | UUUGUUACCUAAUAUAAGAACCGGACAAUUCGAAAGAG | |
| | | UCUCGUCUGUGAGUAGCGAAUCCAAUUCUUGUUUACUC | |
| | | UCUUUGAAUCUGUUCCUAUCAUUGGCAUCUACUAAAAA | |
| | | GACAAUGGCGUCAACAGCAGGGAAGUAGUCCUUCCACA | |
| | | CUCGCCUUGCUGUAGAGAGAAAAAUAAGGUUAGCAUAU | |
| | | UUGGAGACAUUUUUUGUGGAGGAAUCGAAGUUCCUUAA | |
| | | UGUUUUUCGGGGGUAUGAUCUUCUACAAAAAUGAAGGU | |
| | | GAAGCAUGUGAUGUGGACUGGACAUUUCAUUCUAUUGA | |
| | | UGUUUGUUGAAAGAAAUAUACCUGUCCAUGUGUCAAAA | |
| | | UUAUUUUCCGGAAUAAAUAGCCUCGCUGUGGCUGUAAA | |
| | | UUGUAUGUCCGGCGUUUCAUCAACAAUUGC | |
| SAR1 | 373 | CCUUUCCAGUUGUUUGGCCAUACAAGGCAUAGUAAUUU | 24 |
| dsRNA#2 | bases | CUUAGUUCGUCUUCCGAAGCUGCUUGAGGCAAAUCAAU | |
| reverse | | UUUGUUACCUAAUAUAAGAACCGGACAAUUCGAAAGAG | |
| complement | | UCUCGUCUGUGAGUAGCGAAUCCAAUUCUUGUUUACUC | |
| | | UCUUUGAAUCUGUUCCUAUCAUUGGCAUCUACUAAAAA | |
| | | GACAAUGGCGUCAACAGCAGGGAAGUAGUCCUUCCACA | |
| | | CUCGCCUUGCUGUAGAGAGAAAAAUAAGGUUAGCAUAU | |
| | | UUGGAGACAUUUUUUGUGGAGGAAUCGAAGUUCCUUAA | |
| | | UGUUUUUCGGGGGUAUGAUCUUCUACAAAAAUGAAGGU | |
| | | GAAGCAUGUGAUGUGGACUGGACAUUUCAUU | |

### EQUIVALENTS AND SCOPE

In the claims articles such as “a,” “an,” and “the” ˜ may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A polynucleotide that specifically inhibits expression of a Coleopteran vacuolar ATPase-E (vATPase-E) gene.

2. The polynucleotide of claim 1, wherein the Coleoptera vATPase-E gene comprises a deoxynucleic acid (DNA) sequence of SEQ ID NO: 1.

3. The polynucleotide of claim 1 or 2, wherein the polynucleotide is a ribonucleic acid (RNA).

4. The polynucleotide of claim 3, wherein the RNA is a double-stranded RNA (dsRNA) comprising a first strand that is complementary to a messenger RNA (mRNA) encoded by the Coleoptera vATPase-E gene, and a second strand that is complementary to the first strand.

5. The polynucleotide of claim 4, wherein the mRNA comprises the RNA sequence of SEQ ID NO: 2.

6. The polynucleotide of claim 4 or 5, wherein the first strand of the dsRNA comprises an RNA sequence 70% to 100% complementary to the mRNA or a segment of the mRNA encoded by the Coleoptera vATPase-E gene.

7. The polynucleotide of claim 6, wherein the first strand of the dsRNA comprises an RNA sequence 75% to 80%, 80% to 100%, 85% to 100%, 90% to 100%, 95% to 100%, or 98% to 100% complementary to the mRNA or the segment of the mRNA encoded by the Coleoptera vATPase-E gene.

8. The polynucleotide of claim 7, wherein the segment of the mRNA has a length of at least 18 to 500 nucleotides, at least 21 to 500 nucleotides, at least 50 to 500 nucleotides, at least 100 to 500 nucleotides, or at least 200 to 500 nucleotides.

9. The polynucleotide of any one of claims 4-8, wherein the first strand of the dsRNA comprises at least 18 to 21 contiguous nucleotides at least 90% to 100%, 95% to 100%, or at least 98% to 100%, complementary to the mRNA or the segment of the mRNA encoded by the Coleoptera vATPase-E gene.

10. The polynucleotide of any one of claims 4-9, wherein the first strand of the dsRNA comprises an RNA sequence that has 70% to 100% identity to the RNA sequence or a segment of the RNA sequence of any one of SEQ ID NO: 5-7.

11. The polynucleotide of claim 10, wherein the first strand of the dsRNA comprises an RNA sequence that has 75% to 100%, 80% to 100%, 85% to 100%, 90% to 100%, 95% to 100%, or 98% to 100% identity to the RNA sequence or the segment of the RNA sequence of any one of SEQ ID NOS: 5-7.

12. The polynucleotide of claim 10 or 11, wherein the segment of the RNA sequence of any one of SEQ ID NOS: 5-7 has a length of at least 18 to 500 nucleotides, at least 21 to 500 nucleotides, at least 50 to 500 nucleotides, at least 100 to 500 nucleotides, or at least 200 to 500 nucleotides.

13. The polynucleotide of any one of claims 10-12, wherein the first strand of the dsRNA comprises at least 18 to 21 contiguous nucleotides that have at least 90% to 100%, 95% to 100%, or at least 98% to 100%, identity to the RNA sequence of any one of SEQ ID NOS: 5-7.

14. The polynucleotide of claim 2 or 3, wherein the RNA is a single-stranded RNA (ssRNA) that binds to an mRNA encoded by the Coleoptera vATPase-E gene.

15. The polynucleotide of claim 14, wherein the mRNA comprises the RNA sequence of SEQ ID NO: 2.

16. The polynucleotide of claim 14 or 15, wherein the ssRNA comprises an RNA sequence 70% to 100% complementary to the mRNA or a segment of the mRNA encoded by the Coleoptera vATPase-E gene.

17. The polynucleotide of claim 16, wherein the ssRNA comprises an RNA sequence 75% to 100%, 80% to 100%, 85% to 100%, 90% to 100%, 95% to 100%, or 98% to 100% complementary to the mRNA or the segment of the mRNA encoded by the Coleoptera vATPase-E gene.

18. The polynucleotide of claim 17, wherein the segment of the mRNA has a length of at least 18 to 500 nucleotides, at least 21 to 500 nucleotides, at least 50 to 500 nucleotides, at least 100 to 500 nucleotides, or at least 200 to 500 nucleotides.

19. The polynucleotide of any one of claims 14-18, wherein the ssRNA comprises at least 18 to 21 contiguous nucleotides at least 90% to 100%, 95% to 100%, or at least 98% to 100%, complementary to the mRNA.

20. The polynucleotide of any one of claims 14-19, wherein the ssRNA comprises an RNA sequence that has 70% to 100% identity to the RNA sequence of any one of SEQ ID NOS: 5-7.

21. The polynucleotide of claim 20, wherein the ssRNA comprises an RNA sequence that has 75% to 100%, 80% to 100%, 85% to 100%, 90% to 100%, 95% to 100%, or 98% to 100% identity to the RNA sequence or the segment of the RNA sequence of any one of SEQ ID NOS: 5-7.

22. The polynucleotide of claim 21, wherein the segment of the RNA sequence of any one of SEQ ID NOS: 5-7 has a length of at least 18 to 500 nucleotides, at least 21 to 500 nucleotides, at least 50 to 500 nucleotides, at least 100 to 500 nucleotides, or at least 200 to 500 nucleotides.

23. The polynucleotide of any one of claims 20-22, wherein the ssRNA comprises at least 18 to 21 contiguous nucleotides that have at least 90% to 100%, 95% to 100%, or at least 98% to 100%, identity to the RNA sequence of any one of SEQ ID NOS: 5-7.

24. A composition comprising the polynucleotide of any one of claims 1-23.

25. The composition of claim 24, wherein the composition:
(a) further comprises at least one additive selected from the group consisting of: adjuvants, attractants, growth-regulating substances, insect feed, pheromones, proteins, carbohydrates, polymers, organic compounds, biologics, and pesticidal agents;
(b) is formulated as a liquid, a solution, a suspension, an emulsion, an emulsifiable concentrate, a concentrate solution, a low concentrate solution, an ultra-low volume concentrate solution, a water soluble concentrate solution, a bait, an invert emulsion, a flowable, an aerosol, a smoke, a fog, a flowable, a homogenous mixture, a non-homogenous mixture, a solid, a dust, a powder, a granule, a pellet, a capsule, a fumigant, an encapsulated formulation, or a micro-encapsulation formulation; or
(c) is delivered as a spray, fog, seed treatment, drench, drip irrigation, in furrow, insect diet, or bait.

26. The composition of any one of claims 24-25 formulated at a concentration of 0.0001 μg/cm² to 10 μg/cm².

27. A deoxyribonucleic acid (DNA) encoding the RNA of any one of claims 3-23.

28. A method for controlling Coleopteran insect infestation, the method comprising delivering to a plant, ground, a Coleopteran insect, or a diet of a Coleopteran insect with the polynucleotide molecule of any one of claims 1-23 or the composition of any one of claims 24-26.

29. A method for controlling Coleopteran insect infestation, the method comprising delivering to a plant, ground, a Coleopteran insect, or a diet of a Coleopteran insect with a polynucleotide or a composition comprising a polynucleotide that specifically inhibits expression of a Coleopteran vacuolar ATPase-E (vATPase-E) gene.

30. The method of claim 28 or 29, wherein the composition is delivered to a leaf, stem, seed, root, or soil of the plant.

31. The method of any one of claims 28-30, wherein the plant is selected from the group consisting of Solanaceae plants, Brassicaceae plants, Poaceae plants, Cucurbitaceae plants, Fobaceae plants, Apiaceae plants, Amaranthaceae plants, and Malvaceae plants.

32. The method of any one of claims 28-31, wherein the polynucleotide or composition is delivered in an amount sufficient to cause stunting, mortality, decreased feeding, or inhibited reproduction of a Coleopteran insect.

33. The method of any one of claims 28-32, wherein the Coleopteran insect is of a species selected from the group consisting of: Leptinotarsa spp., Phyllotreta spp., Cerotoma spp., Diabrotica spp., Tribolium spp., Anthonomus spp. and Alticini spp.

34. The method of claim 33, wherein the Coleopteran insect is a Leptinotarsa spp. insect.

35. The method of claim 34, wherein the Leptinotarsa spp. insect is a Colorado potato beetle.

36. The method of any one of claims 28-35, wherein the delivering step comprises applying the polynucleotide to the surface of the plant, to the ground, to the Coleopteran insect, or to the diet of a Coleopteran insect at a concentration of at least 0.0001 μg/cm², of 0.0001 μg/cm² to 10 μg/cm², or 0.0001 μg/cm² to 0.1 μg/cm².

37. The method of any one of claims 28-36, wherein percent mortality of Coleopteran insects increases to at least 45% following fewer than 7 days of exposure of the Coleopteran insects to the polynucleotide, relative to a control, optionally under untreated conditions.

38. The method of any one of claims 28-37, wherein percent plant consumption decreases to less than 10% following fewer than 7 days of exposure of Coleopteran insects to the polynucleotide, relative to a control, optionally under untreated conditions; or remains less than 10% following at least 7 days following exposure of Coleopteran insects to the polynucleotide, relative to a control, optionally under untreated conditions.

39. A polynucleotide that specifically inhibits expression of a Coleopteran Proteasome Alpha Type-2 (PTSA2) gene.

40. The polynucleotide of claim 39, wherein the Coleoptera PTSA2 gene comprises a deoxynucleic acid (DNA) sequence of SEQ ID NO: 8 or 9.

41. The polynucleotide of claim 39 or 40, wherein the polynucleotide is a ribonucleic acid (RNA).

42. The polynucleotide of claim 41, wherein the RNA is a double-stranded RNA (dsRNA) comprising a first strand that is complementary to a messenger RNA (mRNA) encoded by the Coleoptera PTSA2 gene, and a second strand that is complementary to the first strand.

43. The polynucleotide of claim 42, wherein the mRNA comprises the RNA sequence of SEQ ID NO: 10 or 11.

44. The polynucleotide of claim 42 or 43, wherein the first strand of the dsRNA comprises an RNA sequence 70% to 100% complementary to the mRNA or a segment of the mRNA encoded by the Coleoptera PTSA2 gene.

45. The polynucleotide of claim 44, wherein the first strand of the dsRNA comprises an RNA sequence 75% to 80%, 80% to 100%, 85% to 100%, 90% to 100%, 95% to 100%, or 98% to 100% complementary to the mRNA or the segment of the mRNA encoded by the Coleoptera PTSA2 gene.

46. The polynucleotide of claim 45, wherein the segment of the mRNA has a length of at least 18 to 500 nucleotides, at least 21 to 500 nucleotides, at least 50 to 500 nucleotides, at least 100 to 500 nucleotides, or at least 200 to 500 nucleotides.

47. The polynucleotide of any one of claims 42-46, wherein the first strand of the dsRNA comprises at least 18 to 21 contiguous nucleotides at least 90% to 100%, 95% to 100%, or at least 98% to 100%, complementary to the mRNA or the segment of the mRNA encoded by the Coleoptera PTSA2 gene.

48. The polynucleotide of any one of claims 42-47, wherein the first strand of the dsRNA comprises an RNA sequence that has 70% to 100% identity to the RNA sequence or a segment of the RNA sequence of any one of SEQ ID NOS: 14-17.

49. The polynucleotide of claim 48, wherein the first strand of the dsRNA comprises an RNA sequence that has 75% to 100%, 80% to 100%, 85% to 100%, 90% to 100%, 95% to 100%, or 98% to 100% identity to the RNA sequence or the segment of the RNA sequence of any one of SEQ ID NOS: 14-17.

50. The polynucleotide of claim 48 or 49, wherein the segment of the RNA sequence of any one of SEQ ID NOS: 14-17 has a length of at least 18 to 500 nucleotides, at least 21 to 500 nucleotides, at least 50 to 500 nucleotides, at least 100 to 500 nucleotides, or at least 200 to 500 nucleotides.

51. The polynucleotide of any one of claims 48-50, wherein the first strand of the dsRNA comprises at least 18 to 21 contiguous nucleotides that have at least 90% to 100%, 95% to 100%, or at least 98% to 100%, identity to the RNA sequence of any one of SEQ ID NOS: 14-17.

52. The polynucleotide of claim 40 or 41, wherein the RNA is a single-stranded RNA (ssRNA) that binds to an mRNA encoded by the Coleoptera PTSA2 gene.

53. The polynucleotide of claim 52, wherein the mRNA comprises the RNA sequence of SEQ ID NO: 10 or 11.

54. The polynucleotide of claim 52 or 53, wherein the ssRNA comprises an RNA sequence 70% to 100% complementary to the mRNA or a segment of the mRNA encoded by the Coleoptera PTSA2 gene.

55. The polynucleotide of claim 54, wherein the ssRNA comprises an RNA sequence 75% to 100%, 80% to 100%, 85% to 100%, 90% to 100%, 95% to 100%, or 98% to 100% complementary to the mRNA or the segment of the mRNA encoded by the Coleoptera PTSA2 gene.

56. The polynucleotide of claim 55, wherein the segment of the mRNA has a length of at least 18 to 500 nucleotides, at least 21 to 500 nucleotides, at least 50 to 500 nucleotides, at least 100 to 500 nucleotides, or at least 200 to 500 nucleotides.

57. The polynucleotide of any one of claims 52-56, wherein the ssRNA comprises at least 18 to 21 contiguous nucleotides at least 90% to 100%, 95% to 100%, or at least 98% to 100%, complementary to the mRNA.

58. The polynucleotide of any one of claims 52-57, wherein the ssRNA comprises an RNA sequence that has 70% to 100% identity to the RNA sequence of any one of SEQ ID NOS: 14-17.

59. The polynucleotide of claim 58, wherein the ssRNA comprises an RNA sequence that has 75% to 100%, 80% to 100%, 85% to 100%, 90% to 100%, 95% to 100%, or 98% to 100% identity to the RNA sequence or the segment of the RNA sequence of any one of SEQ ID NOS: 14-17.

60. The polynucleotide of claim 59, wherein the segment of the RNA sequence of any one of SEQ ID NOS: 14-17 has a length of at least 18 to 500 nucleotides, at least 21 to 500 nucleotides, at least 50 to 500 nucleotides, at least 100 to 500 nucleotides, or at least 200 to 500 nucleotides.

61. The polynucleotide of any one of claims 58-60, wherein the ssRNA comprises at least 18 to 21 contiguous nucleotides that have at least 90% to 100%, 95% to 100%, or at least 98% to 100%, identity to the RNA sequence of any one of SEQ ID NOS: 14-17.

62. A composition comprising the polynucleotide of any one of claims 39-61.

63. The composition of claim 62, wherein the composition:
(a) further comprises at least one additive selected from the group consisting of: adjuvants, attractants, growth-regulating substances, insect feed, pheromones, proteins, carbohydrates, polymers, organic compounds, biologics, and pesticidal agents;
(b) is formulated as a liquid, a solution, a suspension, an emulsion, an emulsifiable concentrate, a concentrate solution, a low concentrate solution, an ultra-low volume concentrate solution, a water soluble concentrate solution, a bait, an invert emulsion, a flowable, an aerosol, a smoke, a fog, a flowable, a homogenous mixture, a non-homogenous mixture, a solid, a dust, a powder, a granule, a pellet, a capsule, a fumigant, an encapsulated formulation, or a micro-encapsulation formulation; or
(c) is delivered as a spray, fog, seed treatment, drench, drip irrigation, in furrow, insect diet, or bait.

64. The composition of any one of claims 62 or 63 formulated at a concentration of 0.0001 μg/cm² to 10 μg/cm².

65. A deoxyribonucleic acid (DNA) encoding the RNA of any one of claims 41-61.

66. A method for controlling Coleopteran insect infestation, the method comprising delivering to a plant, ground, a Coleopteran insect, or a diet of a Coleopteran insect with the polynucleotide molecule of any one of claims 39-61 or the composition of any one of claims 62-64.

67. A method for controlling Coleopteran insect infestation, the method comprising delivering to a plant, ground, a Coleopteran insect, or a diet of a Coleopteran insect with a polynucleotide or a composition comprising a polynucleotide that specifically inhibits expression of a Coleopteran Proteasome Alpha Type-2 (PTSA2) gene.

68. The method of claim 66 or 67, wherein the composition is delivered to a leaf, stem, seed, root, or soil of the plant.

69. The method of any one of claims 66-68, wherein the plant is selected from the group consisting of Solanaceae plants, Brassicaceae plants, Poaceae plants, Cucurbitaceae plants, Fobaceae plants, Apiaceae plants, Amaranthaceae plants, and Malvaceae plants.

70. The method of any one of claims 66-69, wherein the polynucleotide or composition is delivered in an amount sufficient to cause stunting, mortality, decreased feeding, or inhibited reproduction of a Coleopteran insect.

71. The method of any one of claims 66-70, wherein the Coleopteran insect is of a species selected from the group consisting of: Leptinotarsa spp., Phyllotreta spp., Cerotoma spp., Diabrotica spp., Tribolium spp., Anthonomus spp. and Alticini spp.

72. The method of claim 71, wherein the Coleopteran insect is a Leptinotarsa spp. insect.

73. The method of claim 72, wherein the Leptinotarsa spp. insect is a Colorado potato beetle.

74. The method of any one of claims 66-73, wherein the delivering step comprises applying the polynucleotide to the surface of the plant, to the ground, to the Coleopteran insect, or to the diet of a Coleopteran insect at a concentration of at least 0.0001 μg/cm², of 0.0001 μg/cm² to 10 μg/cm², or 0.0001 μg/cm² to 0.1 μg/cm².

75. The method of any one of claims 66-74, wherein percent mortality of Coleopteran insects increases to at least 45% following fewer than 7 days of exposure of the Coleopteran insects to the polynucleotide, relative to a control, optionally under untreated conditions.

76. The method of any one of claims 66-75, wherein percent plant consumption decreases to less than 10% following fewer than 7 days of exposure of Coleopteran insects to the polynucleotide, relative to a control, optionally under untreated conditions; or remains less than 10% following at least 7 days following exposure of Coleopteran insects to the polynucleotide, relative to a control, optionally under untreated conditions.

77. A polynucleotide that specifically inhibits expression of a Coleopteran Secretion Associated Ras Related GTPase 1 (SAR1) gene.

78. The polynucleotide of claim 77, wherein the Coleoptera SAR1 gene comprises a deoxynucleic acid (DNA) sequence of SEQ ID NO: 18.

79. The polynucleotide of claim 77 or 78, wherein the polynucleotide is a ribonucleic acid (RNA).

80. The polynucleotide of claim 79, wherein the RNA is a double-stranded RNA (dsRNA) comprising a first strand that is complementary to a messenger RNA (mRNA) encoded by the Coleoptera SAR1 gene, and a second strand that is complementary to the first strand.

81. The polynucleotide of claim 80, wherein the mRNA comprises the RNA sequence of SEQ ID NO: 19.

82. The polynucleotide of claim 80 or 81, wherein the first strand of the dsRNA comprises an RNA sequence 70% to 100% complementary to the mRNA or a segment of the mRNA encoded by the Coleoptera SAR1 gene.

83. The polynucleotide of claim 82, wherein the first strand of the dsRNA comprises an RNA sequence 75% to 80%, 80% to 100%, 85% to 100%, 90% to 100%, 95% to 100%, or 98% to 100% complementary to the mRNA or the segment of the mRNA encoded by the Coleoptera SAR1 gene.

84. The polynucleotide of claim 83, wherein the segment of the mRNA has a length of at least 18 to 600 nucleotides, at least 21 to 600 nucleotides, at least 50 to 600 nucleotides, at least 100 to 600 nucleotides, or at least 200 to 600 nucleotides.

85. The polynucleotide of any one of claims 80-84, wherein the first strand of the dsRNA comprises at least 18 to 21 contiguous nucleotides at least 90% to 100%, 95% to 100%, or at least 98% to 100%, complementary to the mRNA or the segment of the mRNA encoded by the Coleoptera SAR1 gene.

86. The polynucleotide of any one of claims 80-85, wherein the first strand of the dsRNA comprises an RNA sequence that has 70% to 100% identity to the RNA sequence or a segment of the RNA sequence of any one of SEQ ID NOS: 22-24.

87. The polynucleotide of claim 86, wherein the first strand of the dsRNA comprises an RNA sequence that has 75% to 100%, 80% to 100%, 85% to 100%, 90% to 100%, 95% to 100%, or 98% to 100% identity to the RNA sequence or the segment of the RNA sequence of any one of SEQ ID NOS: 22-24.

88. The polynucleotide of claim 86 or 87, wherein the segment of the RNA sequence of any one of SEQ ID NOS: 22-24 has a length of at least 18 to 600 nucleotides, at least 21 to 600 nucleotides, at least 50 to 600 nucleotides, at least 100 to 600 nucleotides, or at least 200 to 600 nucleotides.

89. The polynucleotide of any one of claims 86-88, wherein the first strand of the dsRNA comprises at least 18 to 21 contiguous nucleotides that have at least 90% to 100%, 95% to 100%, or at least 98% to 100%, identity to the RNA sequence of any one of SEQ ID NOS: 22-24.

90. The polynucleotide of claim 78 or 79, wherein the RNA is a single-stranded RNA (ssRNA) that binds to an mRNA encoded by the Coleoptera SAR1 gene.

91. The polynucleotide of claim 90, wherein the mRNA comprises the RNA sequence of SEQ ID NO: 19.

92. The polynucleotide of claim 90 or 91, wherein the ssRNA comprises an RNA sequence 70% to 100% complementary to the mRNA or a segment of the mRNA encoded by the Coleoptera SAR1 gene.

93. The polynucleotide of claim 92, wherein the ssRNA comprises an RNA sequence 75% to 100%, 80% to 100%, 85% to 100%, 90% to 100%, 95% to 100%, or 98% to 100% complementary to the mRNA or the segment of the mRNA encoded by the Coleoptera SAR1 gene.

94. The polynucleotide of claim 93, wherein the segment of the mRNA has a length of at least 18 to 600 nucleotides, at least 21 to 600 nucleotides, at least 50 to 600 nucleotides, at least 100 to 600 nucleotides, or at least 200 to 600 nucleotides.

95. The polynucleotide of any one of claims 90-94, wherein the ssRNA comprises at least 18 to 21 contiguous nucleotides at least 90% to 100%, 95% to 100%, or at least 98% to 100%, complementary to the mRNA.

96. The polynucleotide of any one of claims 90-95, wherein the ssRNA comprises an RNA sequence that has 70% to 100% identity to the RNA sequence of any one of SEQ ID NOS: 22-24.

97. The polynucleotide of claim 96, wherein the ssRNA comprises an RNA sequence that has 75% to 100%, 80% to 100%, 85% to 100%, 90% to 100%, 95% to 100%, or 98% to 100% identity to the RNA sequence or the segment of the RNA sequence of any one of SEQ ID NOS: 22-24.

98. The polynucleotide of claim 97, wherein the segment of the RNA sequence of any one of SEQ ID NOS: 22-24 has a length of at least 18 to 600 nucleotides, at least 21 to 600 nucleotides, at least 50 to 600 nucleotides, at least 100 to 600 nucleotides, or at least 200 to 600 nucleotides.

99. The polynucleotide of any one of claims 96-98, wherein the ssRNA comprises at least 18 to 21 contiguous nucleotides that have at least 90% to 100%, 95% to 100%, or at least 98% to 100%, identity to the RNA sequence of any one of SEQ ID NOS: 22-24.

100. A composition comprising the polynucleotide of any one of claims 77-99.

101. The composition of claim 100, wherein the composition:
(a) further comprises at least one additive selected from the group consisting of: adjuvants, attractants, growth-regulating substances, insect feed, pheromones, proteins, carbohydrates, polymers, organic compounds, biologics, and pesticidal agents;
(b) is formulated as a liquid, a solution, a suspension, an emulsion, an emulsifiable concentrate, a concentrate solution, a low concentrate solution, an ultra-low volume concentrate solution, a water soluble concentrate solution, a bait, an invert emulsion, a flowable, an aerosol, a smoke, a fog, a flowable, a homogenous mixture, a non-homogenous mixture, a solid, a dust, a powder, a granule, a pellet, a capsule, a fumigant, an encapsulated formulation, or a micro-encapsulation formulation; or
(c) is delivered as a spray, fog, seed treatment, drench, drip irrigation, in furrow, insect diet, or bait.

102. The composition of 100 or 101 formulated at a concentration of 0.0001 μg/cm² to 10 μg/cm².

103. A deoxyribonucleic acid (DNA) encoding the RNA of any one of claims 79-99.

104. A method for controlling Coleopteran insect infestation, the method comprising delivering to a plant, ground, a Coleopteran insect, or a diet of a Coleopteran insect with the polynucleotide molecule of any one of claims 77-99 or the composition of any one of claims 100-102.

105. A method for controlling Coleopteran insect infestation, the method comprising delivering to a plant, ground, a Coleopteran insect, or a diet of a Coleopteran insect with a polynucleotide or a composition comprising a polynucleotide that specifically inhibits expression of a Coleopteran Secretion Associated Ras Related GTPase 1 (SAR1) gene.

106. The method of claim 104 or 105, wherein the composition is delivered to a leaf, stem, seed, root, or soil of the plant.

107. The method of any one of claims 104-106, wherein the plant is selected from the group consisting of Solanaceae plants, Brassicaceae plants, Poaceae plants, Cucurbitaceae plants, Fobaceae plants, Apiaceae plants, Amaranthaceae plants, and Malvaceae plants.

108. The method of any one of claims 104-107, wherein the polynucleotide or composition is delivered in an amount sufficient to cause stunting, mortality, decreased feeding, or inhibited reproduction of a Coleopteran insect.

109. The method of any one of claims 104-108, wherein the Coleopteran insect is of a species selected from the group consisting of: Leptinotarsa spp., Phyllotreta spp., Cerotoma spp., Diabrotica spp., Tribolium spp., Anthonomus spp. and Alticini spp.

110. The method of claim 109, wherein the Coleopteran insect is a Leptinotarsa spp. insect.

111. The method of claim 110, wherein the Leptinotarsa spp. insect is a Colorado potato beetle.

112. The method of any one of claims 104-111, wherein the delivering step comprises applying the polynucleotide to the surface of the plant, to the ground, to the Coleopteran insect, or to the diet of a Coleopteran insect at a concentration of at least 0.0001 μg/cm², of 0.0001 μg/cm² to 10 μg/cm², or 0.0001 μg/cm² to 0.1 μg/cm².

113. The method of any one of claims 104-112, wherein percent mortality of Coleopteran insects increases to at least 45% following fewer than 7 days of exposure of the Coleopteran insects to the polynucleotide, relative to a control, optionally under untreated conditions.

114. The method of any one of claims 104-113, wherein percent plant consumption decreases to less than 10% following fewer than 7 days of exposure of Coleopteran insects to the polynucleotide, relative to a control, optionally under untreated conditions; or remains less than 10% following at least 7 days following exposure of Coleopteran insects to the polynucleotide, relative to a control, optionally under untreated conditions.
